SEQUENCE LISTING (82 sequences)

SEQ ID NO: 1 - protein, 405 aa, synthetic construct
MGSSWSHPQFEKGGGSGGGSGGSAWSHPQFEKMSDSEVNQEAKPEVKPEVHREQIGGSKFNEFKTPQIDPIFDLYVAYGYVESLIRGGAKEATLIPHGASYLIQTDVSNEEFRHGLVDALSEMLSLHIALARHSPREGGKLVSDADFSAGANINNVYWDSVPRNLEKVMKDLEKKRSVKGTATIPITLMPSAGKYMLKHFGVQGGNPIKVDLLNYALAWVGFHYYTPYIKYAKGDTTWIHIYQIAPVEEVDMISILSLKDLKMHLPHYYESNLDFLINRRLALLYHLLHSESLGALELFTEKEFVIHSYTLERSGNNQAIRSFEEEEIGKLMDFLWKLKRRDFYHAIKFIDDLLKKATEGALALIDAIMNERLEGFYTALKLGKKAGVVSSREIVAALEDIICER

SEQ ID NO: 2 - protein, 120 aa, synthetic construct
MGGWIRNIGRYLSYLVDDTFEEYAYDVVDGIAKARTQEELLEGVYKALRLAPKLKKKAESKGCPPPRIPSPEDIEALEEKVEQLSNPKDLRKLAVSLALWAFASWNNCPKKGKGTEGGVE

SEQ ID NO: 3 - protein, 341 aa, synthetic construct
MMYVRISGRIRLNAHSLNAQGGGGTNYIEITKTKVTVRTENGWTVVEVPAITGNMLKHWHFVGFVDYFKTTPYGVNLTERALRYNGTRFGQGETTATKANGATVQLNDEATIIKELADADVHGFLAPKTGRRRVSLVKASFILPTEDFIKEVEGERLITAIKHNRVDVDEKGAIGSSKEGTAQMLFSREYATGLYGFSIVLDLGLVGIPQGLPVKFEENQPRPNIVIDPNERKARIESALKALIPMLSGYIGANLARSFPVFKVEELVAIASEGPIPALVHGFYEDYIEANRSIIKNARALGFNIEVFTYNVDLGEDIEATKVSSVEELVANLVKMVGGKE

SEQ ID NO: 4 - protein, 272 aa, synthetic construct
MDILLVCLRFPFFSVAKRSYQVRTSFLLPPPSALKGALAKGLILLKPEKYASSSLDEAALKAIKEIESKLVDIKAVSVAPLSPLIRNAFLLKRLRNLESGSNAEKSDAMRREYTFTRELLVAYIFKNLTQEEKNLYLKAAMLIDVIGDTESLATPVWASFVKPEDKKAPLAFSAPYTEIYSLLSSKIQAKGKIRMYIEKMRVSPEYSKTKGPQEEIFYLPIEERRYKRIVYYARTIYPPEVEKALTVDGEVLGIWIPKNSSESGSHHHHHHH

SEQ ID NO: 5 - protein, 199 aa, synthetic construct
MYLSKVIIARAWSRDLYQLHQGLWHLFPNRPDAARDFLFHVEKRNTPEGCHVLLQSAQMPVSTAVATVIKTKQVEFQLQVGVPLYFRLRANPIKTILDNQKRLDSKGNIKRCRVPLIKEAEQIAWLQRKLGNAARVEDVHPISERPQYFSGDGKSGKIQTVCFEGVLTINDAPALIDLVQQGIGPAKSMGCGLLSLAPL

SEQ ID NO: 6 - protein, 678 aa, synthetic construct
MGSSHHHHHHSQLEVLFQGPLDTEKLFRELTGFEPYDYQLRAWEKIREIMNNGGKVIIEVPTAGGKTETAVMPFFAGIYNNNWPVARLVYVLPTRSLVEKQAERLRNLVYKLLQLKGKSKEEAEKLARELVVVEYGLEKTHAFLGWVVVTTWDAFLYGLAAHRTVGNRFTFPAGAIAQSLVIFDEVQMYQDESMYMPRLLSLVVGILEEANVPLVIMSATIPSKLREMIAGDTEVITVDKNDKNKPSRGNVKVRLVEGDITDVLNDIKKILKNGKKVLVVRNTVRKAVETYQVLKKKLNDTLANPSDALLIHSRFTIGDRREKERALDSARLIVATQVVEAGLDLPNVGLVVTDIAPLDALIQRIGRCARRPGEEGEGIILIPVENCIEHEKIVRGLSELMEKIGEDTVVFATVTSTNEYDRVVEIHYGEGKKNFVYVGDIDTARRVLEKKRSKKLPKDLYIIPYSVSPYDPLVLLTTYDELSKIGEYLADTTKARKALDRVYKFHYENNIVPKEFASAYIYFKELKLFSAPPEYELRSRPELYVLLYPMNIEKNERVEDKVIDNLETARIIRISYSVKEWKKSDVVIGRLMKEWDKNAEKWVWKVRKSFKIDPYEIYVIDAKYYNSELGFITNLSDTNSHTDSDSKVRTRNSEHSSKKNRSKGKKGQTSLENWGVRV

SEQ ID NO: 7 - protein, 259 aa, synthetic construct
MGSSHHHHHHSQLEVLFQGPLGSSCKAFQGQTLREHIEAMLAAWEIVKNKYIPSIIRVMKTVGVKFTEEDADKFMKTLIILHDVGKCSEVYQKHLSNNEPLRGFRHELVSAYYAYNILKDMFKDETIAFIGALVVMMHHEPILMGQIRSLDKEELTPEVVLDKLRTFNGVMEGTESFIKSMIKEKLGVIPKVPSPTQEDVLREVIRLSVLARHRPDSGKLRMVVGALLIPLVLCDYKGAKEREGESPKFAEVLRVEMMK

SEQ ID NO: 8 - protein, 348 aa, Pyrococcus furiosus
MKFNEFKTPQIDPIFDLYVAYGYVESLIRGGAKEATLIPHGASYLIQTDVSNEEFRHGLVDALSEMLSLHIALARHSPREGGKLVSDADFSAGANINNVYWDSVPRNLEKVMKDLEKKRSVKGTATIPITLMPSAGKYMLKHFGVQGGNPIKVDLLNYALAWVGFHYYTPYIKYAKGDTTWIHIYQIAPVEEVDMISILSLKDLKMHLPHYYESNLDFLINRRLALLYHLLHSESLGALELFTEKEFVIHSYTLERSGNNQAIRSFEEEEIGKLMDFLWKLKRRDFYHAIKFIDDLLKKATEGALALIDAIMNERLEGFYTALKLGKKAGVVSSREIVAALEDIICER

SEQ ID NO: 9 - protein, 263 aa, Pyrococcus furiosus
MDILLVCLRFPFFSVAKRSYQVRTSFLLPPPSALKGALAKGLILLKPEKYASSSLDEAALKAIKEIESKLVDIKAVSVAPLSPLIRNAFLLKRLRNLESGSNAEKSDAMRREYTFTRELLVAYIFKNLTQEEKNLYLKAAMLIDVIGDTESLATPVWASFVKPEDKKAPLAFSAPYTEIYSLLSSKIQAKGKIRMYIEKMRVSPEYSKTKGPQEEIFYLPIEERRYKRIVYYARTIYPPEVEKALTVDGEVLGIWIPKNSSES

SEQ ID NO: 10 - protein, 340 aa, Pyrococcus furiosus
MYVRISGRIRLNAHSLNAQGGGGTNYIEITKTKVTVRTENGWTVVEVPAITGNMLKHWHFVGFVDYFKTTPYGVNLTERALRYNGTRFGQGETTATKANGATVQLNDEATIIKELADADVHGFLAPKTGRRRVSLVKASFILPTEDFIKEVEGERLITAIKHNRVDVDEKGAIGSSKEGTAQMLFSREYATGLYGFSIVLDLGLVGIPQGLPVKFEENQPRPNIVIDPNERKARIESALKALIPMLSGYIGANLARSFPVFKVEELVAIASEGPIPALVHGFYEDYIEANRSIIKNARALGFNIEVFTYNVDLGEDIEATKVSSVEELVANLVKMVGGKE

SEQ ID NO: 11 - protein, 120 aa, Pyrococcus furiosus; identical to SEQ ID NO: 2

SEQ ID NO: 12 - protein, 237 aa, Pyrococcus furiosus
MSCKAFQGQTLREHIEAMLAAWEIVKNKYIPSIIRVMKTVGVKFTEEDADKFMKTLIILHDVGKCSEVYQKHLSNNEPLRGFRHELVSAYYAYNILKDMFKDETIAFIGALVVMMHHEPILMGQIRSLDKEELTPEVVLDKLRTFNGVMEGTESFIKSMIKEKLGVIPKVPSPTQEDVLREVIRLSVLARHRPDSGKLRMVVGALLIPLVLCDYKGAKEREGESPKFAEVLRVEMMK

SEQ ID NO: 13 - protein, 658 aa, Pyrococcus furiosus
MDTEKLFRELTGFEPYDYQLRAWEKIREIMNNGGKVIIEVPTAGGKTETAVMPFFAGIYNNNWPVARLVYVLPTRSLVEKQAERLRNLVYKLLQLKGKSKEEAEKLARELVVVEYGLEKTHAFLGWVVVTTWDAFLYGLAAHRTVGNRFTFPAGAIAQSLVIFDEVQMYQDESMYMPRLLSLVVGILEEANVPLVIMSATIPSKLREMIAGDTEVITVDKNDKNKPSRGNVKVRLVEGDITDVLNDIKKILKNGKKVLVVRNTVRKAVETYQVLKKKLNDTLANPSDALLIHSRFTIGDRREKERALDSARLIVATQVVEAGLDLPNVGLVVTDIAPLDALIQRIGRCARRPGEEGEGIILIPVENCIEHEKIVRGLSELMEKIGEDTVVFATVTSTNEYDRVVEIHYGEGKKNFVYVGDIDTARRVLEKKRSKKLPKDLYIIPYSVSPYDPLVLLTTYDELSKIGEYLADTTKARKALDRVYKFHYENNIVPKEFASAYIYFKELKLFSAPPEYELRSRPELYVLLYPMNIEKNERVEDKVIDNLETARIIRISYSVKEWKKSDVVIGRLMKEWDKNAEKWVWKVRKSFKIDPYEIYVIDAKYYNSELGFITNLSDTNSHTDSDSKVRTRNSEHSSKKNRSKGKKGQTSLENWGVRV

SEQ ID NO: 14 - protein, 199 aa, Pyrococcus furiosus; identical to SEQ ID NO: 5

SEQ ID NO: 15 - RNA, 66 nt, synthetic construct
  source 1..66: mol_type = other RNA
attgaaagacagctccaactaccacaagtttatattcagtcatttgagttccccgcgccagcgggg

SEQ ID NO: 16 - RNA, 66 nt, synthetic construct
  source 1..66: mol_type = other RNA
attgaaagtcagctccaactaccacaagtttatattcagtcatttgagttccccgcgccagcgggg

SEQ ID NO: 17 - DNA, 35 bp, synthetic construct
  source 1..35: mol_type = other DNA
atatggatccatggggggatggatcagaaatattg

SEQ ID NO: 18 - DNA, 34 bp, synthetic construct
  source 1..34: mol_type = other DNA
atatctcgagtcagctctctgaagagtttttcgg

SEQ ID NO: 19 - DNA, 37 bp, synthetic construct
  source 1..37: mol_type = other DNA
atatggatccatgaaatttaacgaatttaaaacacct

SEQ ID NO: 20 - DNA, 35 bp, synthetic construct
  source 1..35: mol_type = other DNA
atatctcgagttaacgttcacatattatatcctcc

SEQ ID NO: 21 - DNA, 36 bp, synthetic construct
  source 1..36: mol_type = other DNA
atatggatccatgaggtttttaataagactagttcc

SEQ ID NO: 22 - DNA, 36 bp, synthetic construct
  source 1..36: mol_type = other DNA
atatctcgagttatactccagtttttaattcctctc

SEQ ID NO: 23 - DNA, 37 bp, synthetic construct
  source 1..37: mol_type = other DNA
gattcaggatccatgtatctcagtaaagtcatcattg

SEQ ID NO: 24 - DNA, 34 bp, synthetic construct
  source 1..34: mol_type = other DNA
gtagttctcgagtcacagtggagccaaagatagc

SEQ ID NO: 25 - DNA, 88 bp, synthetic construct
  source 1..88: mol_type = other DNA
gttccaataagactacaaaagaattgaaagagtgcttccccaaacccttaactggttgtaacagttggttccaataagactacaaaag

SEQ ID NO: 26 - DNA, 204 bp, synthetic construct
  source 1..204: mol_type = other DNA
caactttcaattgatgagtccgtgaggacgaaacggtacccggtaccgtcattgaaagttgagtgcttccccaaacccttaactggttgtaacagttgagagttccccgcgccagcggggataaaccgtggcagtcctgttgaaaaacagagaagccaaccagagaaacacacgttgtggtatattacctggtaagctatctaa

SEQ ID NO: 27 - DNA, 87 bp, synthetic construct
  source 1..87: mol_type = other DNA
gagttccccgcgccagcggggattgaaagagtgcttccccaaacccttaactggttgtaacagttggagttccccgcgccagcgggg

SEQ ID NO: 28 - DNA, 65 bp, synthetic construct
  source 1..65: mol_type = other DNA
gccgtacctctgaatcacgaaggggtttgggaattgaccaacattgtcaacgggagttactcgcg

SEQ ID NO: 29 - DNA, 65 bp, synthetic construct
  source 1..65: mol_type = other DNA
cgcgagtaactcccagtgcttccccaaacccttaactggttgtaacagttgttcagaggtacggc

SEQ ID NO: 30 - DNA, 65 bp, synthetic construct
  source 1..65: mol_type = other DNA
gccgtacctctgaatcacgaaggggtttgggaattgaccaacattgtcaacgcaagttactcgcg

SEQ ID NO: 31 - DNA, 65 bp, synthetic construct
  source 1..65: mol_type = other DNA
cgcgagtaacttgcagtgcttccccaaacccttaactggttgtaacagttgttcagaggtacggc

SEQ ID NO: 32 - DNA, 65 bp, synthetic construct
  source 1..65: mol_type = other DNA
gccgtacctctgaatcacgaaggggtttgggaattgaccaacattgtcaactgcagttactcgcg

SEQ ID NO: 33 - DNA, 65 bp, synthetic construct
  source 1..65: mol_type = other DNA
cgcgagtaactgcaagtgcttccccaaacccttaactggttgtaacagttgttcagaggtacggc

SEQ ID NO: 34 - DNA, 33 bp, synthetic construct
  source 1..33: mol_type = other DNA
atatatggatccagttgtaaggcattccaagga

SEQ ID NO: 35 - DNA, 36 bp, synthetic construct
  source 1..36: mol_type = other DNA
gatatgtcgactcacttcatcatttcaactctaaga

SEQ ID NO: 36 - DNA, 32 bp, synthetic construct
  source 1..32: mol_type = other DNA
atatatgtcgactcatactctcaccccccagt

SEQ ID NO: 37 - DNA, 35 bp, synthetic construct
  source 1..35: mol_type = other DNA
atatggatccatggataccgaaaaactcttcagag

SEQ ID NO: 38 - DNA, 60 bp, synthetic construct
  source 1..60: mol_type = other DNA
cgcgagtaactcccttgtagtatgcggtccttgcggctgagagcacttcagaggtacggc

SEQ ID NO: 39 - DNA, 23 bp, synthetic construct
  source 1..23: mol_type = other DNA
cccagagcagggccttagggaag

SEQ ID NO: 40 - DNA, 19 bp, synthetic construct
  source 1..19: mol_type = other DNA
gaaagatggccgcttcccg

SEQ ID NO: 41 - DNA, 19 bp, synthetic construct
  source 1..19: mol_type = other DNA
cagacccaggagtccaggc

SEQ ID NO: 42 - DNA, 22 bp, synthetic construct
  source 1..22: mol_type = other DNA
gctgggaccaccttatattccc

SEQ ID NO: 43 - DNA, 20 bp, synthetic construct
  source 1..20: mol_type = other DNA
agggatcctgtgtggccatc

SEQ ID NO: 44 - DNA, 21 bp, synthetic construct
  source 1..21: mol_type = other DNA
tgcgatgtccggagaggatgg

SEQ ID NO: 45 - skipped

SEQ ID NO: 46 - DNA, 26 bp, synthetic construct
  source 1..26: mol_type = other DNA
agggaaacctttgggcccaattccgg

SEQ ID NO: 47 - DNA, 37 bp, synthetic construct
  source 1..37: mol_type = other DNA
tgagcaaagaccccaacgagaagcgcgatcacatggt

SEQ ID NO: 48 - DNA, 37 bp, synthetic construct
  source 1..37: mol_type = other DNA
aggatgttgccgtcctccttgaagtcgatgcccttca

SEQ ID NO: 49 - DNA, 37 bp, synthetic construct
  source 1..37: mol_type = other DNA
catctctttacaggaccctgggttcccttaccctttg

SEQ ID NO: 50 - DNA, 37 bp, synthetic construct
  source 1..37: mol_type = other DNA
tgtcaattccttcccaattagtatcttctggtgggcc

SEQ ID NO: 51 - DNA, 37 bp, synthetic construct
  source 1..37: mol_type = other DNA
cagttatatgtggaacgaagggtgctaggagaagcat

SEQ ID NO: 52 - DNA, 46 bp, synthetic construct
  source 1..46: mol_type = other DNA
aggaccatgtgatcgcgcttctcgttggggtctttgctcagggcgg

SEQ ID NO: 53 - DNA, 46 bp, synthetic construct
  source 1..46: mol_type = other DNA
ccgccctgagcaaagaccccaacgagaagcgcgatcacatggtcct

SEQ ID NO: 54 - DNA, 46 bp, synthetic construct
  source 1..46: mol_type = other DNA
tgccccaggatgttgccgtcctccttgaagtcgatgcccttcagct

SEQ ID NO: 55 - DNA, 46 bp, synthetic construct
  source 1..46: mol_type = other DNA
agctgaagggcatcgacttcaaggaggacggcaacatcctggggca

SEQ ID NO: 56 - DNA, 34 bp, synthetic construct
  source 1..34: mol_type = other DNA
cacctacggcaagctgttgccagaacctctaagg

SEQ ID NO: 57 - DNA, 34 bp, synthetic construct
  source 1..34: mol_type = other DNA
ccgccagaacacagggagaggatcctgggaggga

SEQ ID NO: 58 - DNA, 34 bp, synthetic construct
  source 1..34: mol_type = other DNA
gacgacggcaactacaagacggaagggggggatg

SEQ ID NO: 59 - DNA, 34 bp, synthetic construct
  source 1..34: mol_type = other DNA
ccgacaagcagaagaacggctgtgccttctagtt

SEQ ID NO: 60 - protein, 78 aa, synthetic construct
IGKLMDFLWKLKRRDFYHAIKFIDDLLKKATEGALALIDAIMNERLEGFYTALKLGKKAGVVSSREIVAALEDIICER

SEQ ID NO: 61 - protein, 105 aa, synthetic construct
IRNIGRYLSYLVDDTFEEYAYDVVDGIAKARTQEELLEGVYKALRLAPKLKKKAESKGCPPPRIPSPEDIEALEEKVEQLSNPKDLRKLAVSLALWAFASWNNCP

SEQ ID NO: 62 - protein, 78 aa, synthetic construct; identical to SEQ ID NO: 60

SEQ ID NO: 63 - protein, 93 aa, synthetic construct
LPVVWDFIGYLKARAFYETMKFVKILNKLERTIKESQEDILREISHQLYSALTNRDEMGFYSAFRVLKRAGYPLDPELVNAIAQYLSEREVIR

SEQ ID NO: 64 - protein, 86 aa, synthetic construct
REGGKLVSDADFSAGANINNVYWDSVPRNLEKVMKDLEKKRSIPITLMPSAGKYMLKHFGVQGGNPIKVDLLNYALAWVGFHYYTP

SEQ ID NO: 65 - protein, 86 aa, synthetic construct; identical to SEQ ID NO: 64

SEQ ID NO: 66 - protein, 86 aa, synthetic construct
REGGKLVSDADFSAGANINNVYWDSIPRNLKKAKERLEAGKLMPITLMPSAGKYMPKHFGAQGGNPIKVDLLNYALAWVGFHYYTP

SEQ ID NO: 67 - protein, 86 aa, synthetic construct
REGGKVISDADFSAGANINNAYWDSVPSRLSTVLEKIVANKKVPITLMPSAGKFLPKHMGIQGGNPLKIDSLSYALAWVGFHYYAP

SEQ ID NO: 68 - protein, 87 aa, synthetic construct
SREGVKVTSDADFSAGANVNNTYWDSVPSRLRDVREKLKEGKKLPITLMPSAGKFIPKHMGVQGGNPLKIDPLNYALAWVGFHYYTP

SEQ ID NO: 69 - protein, 88 aa, synthetic construct
TKEDIKIISDVDFSAGANINNVYWDGIPKTLEKIKENIKKGKLSVPLTLMPAAGKYMPKIYGVKGGNPIKIDDSNYALAWIGFHYYAP

SEQ ID NO: 70 - protein, 44 aa, synthetic construct
VSVAPLSPLIRNAFLLKRLRNLESGSNAEKSDAMRREYTFTREL

SEQ ID NO: 71 - protein, 44 aa, synthetic construct; identical to SEQ ID NO: 70

SEQ ID NO: 72 - protein, 44 aa, synthetic construct
VSVAPISPLVKNAFLLKRLRNLESGSKAEKGDAMRREYTFTREL

SEQ ID NO: 73 - protein, 44 aa, synthetic construct
VGVAPLTPMVKTAFLLKRLRNLEKGSKPEKDDAMRREYVFTKEL

SEQ ID NO: 74 - protein, 44 aa, synthetic construct
VSAAPLGPLVKNAFLLKRLRNLESEKNPEKDDAMRREYVFTTEL

SEQ ID NO: 75 - protein, 42 aa, synthetic construct
KPYKSSIIKTPILLKRLRNLEDQKNPEKSDAMRREYVFAREI

SEQ ID NO: 76 - RNA, 95 nt, synthetic construct
  source 1..95: mol_type = other RNA
gagttccccgcgccagcggggataaaccgnnnnnnnnnnnnnnnnnnnnnnnnnnnnnnnnnnnnngccccgcgccagcggggagttataaaccg

SEQ ID NO: 77 - DNA, 30 bp, synthetic construct
  source 1..30: mol_type = other DNA
gttccaataagactacaaaagaattgaaag

SEQ ID NO: 78 - DNA, 29 bp, synthetic construct
  source 1..29: mol_type = other DNA
gagttccccgcgccagcggggataaaccg

SEQ ID NO: 79 - DNA, 29 bp, synthetic construct
  source 1..29: mol_type = other DNA
gagttccccgcgccagcggggattgaaag

SEQ ID NO: 80 - RNA, 67 nt, synthetic construct
  source 1..67: mol_type = other RNA
attgaaagttgtagtatgcggtccttgcggctgagagcacttcaggagttgcccggccagcgggcca

SEQ ID NO: 81 - DNA, 60 bp, synthetic construct
  source 1..60: mol_type = other DNA
gccgtacctctgaagtgctctcagccgcaaggaccgcatactacaagggagttactcgcg

SEQ ID NO: 82 - DNA, 60 bp, synthetic construct; identical to SEQ ID NO: 38
  source 1..60: mol_type = other DNA

(19) United States
(12) Patent Application Publication
(10) Pub. No.: US 2024/0376454 A1
(43) Pub. Date: Nov. 14, 2024

(54) TYPE I-A CRISPR-CAS3 SYSTEM FOR GENOME EDITING AND DIAGNOSTICS

(71) Applicant: Cornell University, Ithaca, NY (US)

(72) Inventors: Ailong KE, Ithaca, NY (US); Chunyi HU, Singapore (SG)

(21) Appl. No.: 18/684,648

(22) PCT Filed: Aug. 18, 2022
(86) PCT No.: PCT/US2022/075151

§ 371 Date: Feb. 16, 2024

Related U.S. Application Data
(60) Provisional application No. 63/349,929, filed on Jun. 7, 2022.
(60) Provisional application No. 63/349,925, filed on Jun. 7, 2022.
(60) Provisional application No. 63/234,607, filed on Aug. 18, 2021.
(60) Provisional application No. 63/234,597, filed on Aug. 18, 2021.

(51) Int. Cl. C12N 9/22; C12N 15/11; C12N 15/90; C12Q 1/6886

(52) U.S. Cl. CPC C12N 9/22; C12N 15/11; C12N 15/902; C12Q 1/6886; C12N 2310/20; C12Q 2600/156

(57) ABSTRACT

Provided are compositions, methods, and kits for CRISPR-based editing of DNA targets by Type LA CRISPR-associated (Cas) enzymes using a chimeric guide RNA. Nucleic acid diagnostic compositions, methods and kits that include the Type LA CRISPR enzymes and the chimeric guide RNA with a detectably labeled DNA substrate are also provided. The Type I-A CRISPR enzymes may be used with a Cas6 enzyme that encoded by a prokaryotic organisms that does not harbor a Type LA CRISPR system.

### CROSS-REFERENCE TO RELATED APPLICATIONS

This application claims priority to U.S. provisional patent application No. 63/234,597, filed Aug. 18, 2021, and to U.S. provisional patent application No. 63/234,607, filed Aug. 18, 2021, and to U.S. provisional patent application No. 63/349,925, filed Jun. 7, 2022, and to U.S. provisional patent application No. 63/349,929, filed Jun. 7, 2022, the entire disclosures of which are incorporated herein by reference.

### STATEMENT REGARDING FEDERALLY SPONSORED RESEARCH

This invention was made with government support under grant no. 137586 awarded by the National Institutes of Health. The government has certain rights in the invention.

### SEQUENCE LISTING

The instant application contains a Sequence Listing which has been submitted in .xml format and is hereby incorporated by reference in its entirety. Said .xml copy was created on Aug. 15, 2022, is named “TYPE_I-A CRISPR-Cas3_SYSTEM_FOR_GENOME_EDITING_AND_DIAGNOSTICS-ST26Sequencelisting.xml” and is 78,856 bytes in size.

### FIELD OF THE DISCLOSURE

The present disclosure is related to compositions and methods use in modifying DNA using clustered regularly interspaced short palindromic repeats (CRISPR) Type I-A Cas3 systems, and nucleic acid diagnostic approaches using the same.

### BACKGROUND OF THE DISCLOSURE

There is an ongoing and unmet need for improvements in CRISPR-Cas targeting and editing, and for improved nucleic acid diagnostic approaches. The present disclosure is pertinent to these needs.

### BRIEF SUMMARY

The present disclosure demonstrates Type I-A CRISPR-Cas3 systems and derivatives thereof, with certain modifications as further described herein, effectively introduce a spectrum of long-range chromosomal deletions using a single guide RNA in any cell type, including but not limited to human cells. The described systems comprise proteins that are have at least 85% amino acid similarity to described Pyrococcus furiosus (P. furiosus) proteins. P. furiosus is also referred to herein as Pfu and Pfu. The Pfu proteins assemble on a multi-subunit ribonucleoprotein (RNP) complex Cascade to identify DNA targets, and the helicase-nuclease enzyme Cas3 to degrade DNA processively. With various types of delivery approaches for P. furiosus Cascade and Cas3, with the exception of a requirement for a heterologous Cas6, we obtained improved editing efficiency compared to previous systems. The described systems also involve use of a chimeric single guide RNA. In embodiments, the chimeric guide RNA is processed using a guide RNA precursor that is not produced by P. furiosus. In certain embodiments, the chimeric guide RNA comprises a P. furiosus encoded 5′-handle sequence, and a 3′-handle sequence that is not encoded by P. furiosus. In embodiments, if a guide RNA precursor is used to produce the chimeric guide RNA, the precursor is not produced using P. furiosus. In non-limiting embodiments, once assembled, the described systems are capable of functioning at temperatures that are the same as human body temperature, and higher temperatures, such as 45-85° C.

Proteins used in the described systems, compositions and methods comprise Pfu Cascade or the described derivatives thereof, and Pfu Cas3 or the described derivatives thereof. In this regard, Pfu Cascade contains one copy of Cas8a, one copy of Cas5a, and multiple copies of Cas7a and Cas11a. Pfu Cas3″ and Cas3′ subunits assemble into functional Cas3 protein after expression. In embodiments, Cas3″ (referred to as “HD” or the nuclease domain) and Cas3′ (referred to as the helicase domain) are two domains of single Cas3 protein. Pfu Cascade and Cas3 assemble at 1:1 molar ratio into an integral effector complex, which in certain described embodiments also includes a heterologous Cas6. The heterologous Cas6 can be an optional component in certain examples, such as where a ribonucleoprotein (RNP) is assembled prior to being exposed to a DNA substrate. Further, the Cas6 protein is an optional component of the described diagnostic aspect of the described compositions, systems and methods.

In embodiments, for DNA editing, the editing comprises bi-directional deletion. The editing is therefore suitable for targeting and degradation of any site, including but not limited to sites located on chromosomes and extrachromosomal elements, the latter including but not limited to a variety of ectopic viruses, such as viruses that are present and/or replicate in cytoplasm. In embodiments, the bi-directional deletion comprise a sequence comprising an integrated viral sequence. The described systems are also suitable for degradation of DNA segments that contain disease causing genes and/or their regulatory elements. The described systems are configurable to be specific for any DNA sequence that comprises a suitable protospacer adjacent motif (PAM). In an embodiment, a suitable PAM comprises 5′-Y₋₃C₋₂N₋₁, wherein there is a pyrimidine at PAM-3, a cytosine at PAM-2, and any nucleotide at PAM-1. In embodiments, the described systems are configured so that they can bi-directionally delete a strand of DNA that is linked to any marker, such as a single nucleotide polymorphism (SNP), triplet repeats that are associated with certain disorders, and insertions or deletions (indels) that affect open reading frames, which may also be associated with certain disorders. In embodiments, the DNA that is deleted is linked to an inherited disease associated gene. In embodiments, the DNA that is deleted is linked to an integrated viral sequence.

In embodiments, the disclosure provides for use of P. furiosus proteins, or derivatives thereof, for use in modifying DNA, such as chromosomal DNA, or extrachromosomal dsDNA, and for nucleic acid diagnostic tests. In embodiments, the disclosure provides a method of modifying DNA in eukaryotic cells by introducing into the eukaryotic cells: (i) a combination of proteins comprising P. furiosus proteins or proteins that are at least 85% identical to the described P. furiosus proteins, i.e., each protein comprising an amino acid sequence that is at least 85% homologous across its entire length to a P. furiosus protein, and optionally a heterologous Cas6 protein; (ii) a guide RNA (a targeting RNA) comprising a sequence that is complementary to a targeted site in a segment of the DNA, the targeted site comprising a spacer sequence; and (iii) allowing the combination of the proteins and the guide RNA to modify the DNA by bi-directional deletion of a single strand of the DNA. The method, among other properties described herein, leaves the targeted site intact. In embodiments, long bi-directional deletions, such as up to 100 kb, are introduced.

The methods provide for modifying DNA in a population of cells, such as a population of eukaryotic cells an in vitro cell culture. For example, in certain embodiments, a DNA segment is modified in 10%-100% of the cells in an in vitro cell culture, or in 10%-100% of the cells that receive the described system. In embodiments, a bi-directional deletion is made in the described percentages of cells. For example, see FIGS. 7E and 7H.

In certain embodiments, use of the described system produces a bi-directional deletion upstream and downstream of a targeted site that each comprise a deletion of from about 500 base pairs to about 100,000 base pairs. The disclosure further comprises modifying DNA in eukaryotic cells by introducing a DNA repair template, such that the sequence of the DNA repair template is incorporated into a chromosome. For example, single-stranded DNA may be exposed during Cascade-Cas3 mediated DNA degradation, which can allow gene conversion by introducing a DNA repair template, such that the sequence of the DNA repair template is incorporated into a chromosome. This approach can be used for a variety of purposes, such as introducing mutations, indels, and gene conversion approaches. The described systems can be introduced into the cells using a variety of approaches, such as by using mRNA, or a ribonucleoprotein (RNP) complex, or plasmids or other expression vectors, or combinations thereof. The disclosure includes modified eukaryotic cells made by the described methods, and non-human animals comprising or produced from the cells.

The disclosure also provides kits which may comprise combination of recombinant proteins, and/or or one or more polynucleotides that can express a combination of proteins.

In another aspect the disclosure provides an in vitro method for nucleic acid detection. The method comprises contacting a sample that comprises or is suspected of comprising a nucleic acid with a described system and a chimeric guide RNA that is targeted to a single stranded (ss) DNA reporter. The ssDNA reporter comprises a detectable label that is quenched by a quencher moiety. A signal from the label is detectable due to cleavage of the ssDNA reporter by a described Cas3 complex if the nucleic acid is present in the sample.

### BRIEF DESCRIPTION OF THE FIGURES

FIG. 1. Pfu Cascade-Cas3 form an integral effector complex. (A) Arrangement of the Pyrococcus furiosus Type I-A CRISPR-cas operon. (B) Agarose EMSA showing that Pfu Cas3 significantly improved the DNA target binding behavior of Pfu Cascade. (C) Agarose EMSA using differentially labeled binding partners to reveal that I-A Pfu Cas3 bound constitutively to Pfu Cascade. In contrast, I-E Thermobifida fusca (Tfu) Cas3 only interacted with Tfu Cascade upon full R-loop formation. Cas3, Cascade, and dsDNA were labeled with Cy3, Cy5, and FAM, respectively. Overlapping colors indicate complex formation that survived electrophoresis.

FIG. 2. Overview of four cryo-EM snapshots of Pfu Cascade-Cas3 in different functional states. Schematics of the depicted functional state, cryo-EM density, and cartoon representation of the molecular structure of (A) apo Pfu Cascade, (B) Pfu Cascade-Cas3, (C) Pfu Cascade-Cas3 opening a partial R-loop (unwinding 17 bp of PAM-proximal dsDNA target), and (D) Pfu Cascade-Cas3 opening a full R-loop (unwinding 37 bp of PAM-proximal dsDNA target). Refer the coloring scheme from the schematics.

FIG. 3. Pfu Cas3 rigidifies the PAM-recognition subunit of Pfu Cascade, enabling DNA target-binding. (A) Further classification revealed four 3D variants from the apo Pfu Cascade cryo-EM reconstruction, each represents the specified proportion of the total particles. They vary in the Cas8a NTD density. Only ˜10% particles contain choppy densities large enough to cover entire Cas8a. In contrast, Cas8a NTD density is well defined in the Pfu Cascade-Cas3 reconstruction. (B) Local resolution estimate based on the per-residue r.m.s.d. value. Cas8a NTD and the rest of the inner belly subunits in the apo Pfu Cascade have reduced resolution and elevated motion based on this analysis. In contrast, the equivalent subunits in the Pfu Cascade-Cas3 structure are resolved at the same resolution as the rest of the structure. (C) Detailed molecular contacts between Pfu Cas3 and Pfu Cascade. An orientation view is provided to the left. The boxed regions are analyzed in zoom-in panels to the right. (D) Native-agarose EMSA showing that when the molecular contact is disrupted, by the V187E mutation to Cas3 HD, Cas3 can no longer improve the target DNA binding behavior of Cascade as the wild-type Cas3 does.

FIG. 4. PAM recognition mechanism. (A, B) Two zoom-in views of the PAM-recognition mechanism by Pfu Cascade-Cas3. Coloring scheme is consistent with FIG. 3C. (C) Diagram of the PAM recognition contacts. (D) The impact of disrupting the observed PAM contacts on target binding affinity, evaluated using native-agarose EMSA.

FIG. 5. Structural basis for the allosteric activation of Pfu Cas3 nuclease upon full R loop formation by Pfu Cascade-Cas3. (A) Side-by-side comparison of the Pfu Cascade-Cas3 structure before, during and after R-loop formation, which reveals the timing and the nature of the conformational change during the R-loop formation process. Cas3 HD-nuclease center is only exposed upon full R-loop formation (note the accessibility of the two catalytic metal ions in dark balls). NTS DNA has been nicked and threaded through the Cas3 helicase, ready for processive degradation by the Cas3 HD-nuclease. (B) Zoom-in of the Cas3 HD-nuclease center depicting the unlatching movements of loops L1, L2, and Lc upon R-loop formation. Together the loops form an R-loop dependent conformational switch. (C) Denaturing-PAGE revealing the R-loop dependent activation of the nuclease activity in Cas3. Upon activation, Pfu Cascade-Cas3 not only cleaves the cognate DNA target, but also the fluorescent ssDNA reporter nearby. Cy5-labeled target strand is colored in red, Cy3-labeled non-target strand in green, FAM-labeled ssDNA reporter in white color. (D) Nuclease activity changes as assayed in (C), quantified from the intensity of the full-length fluorescent ss-DNA reporter band in each assay condition. (E) Denaturing-PAGE showing the loss of autoinhibition of the Cas3 nuclease activity inside Pfu Cascade-Cas3 when the conformational switch was disrupted (ΔL1, ΔL2). Coloring scheme is the same to (C). (F) Quantification of the nuclease activity as assayed in (E).

FIG. 6. Mechanism-inspired development of HASTE nucleic acid detection platform. (A) Diagram of the nucleic acid detection platform, based on the nuclease activity changes inside Pfu Cascade-Cas3 in response to cognate and non-cognate DNA targets. F, fluorophore; Q, quencher. (B) Reagent combinations that lead to clean background and robust positive signal in test tubes. (C) Normalized fluorescence changes when four different poly-deoxynucleotide ssDNA-FQ reporters were used. (D) Real time fluorescent changes as the result of ssDNA-FQ cleavage by Pfu Cascade-Cas3 as it encounters a cognate DNA target, a non-cognate target, or none. Cas3 alone is not autoinhibited. Its strong collateral cleavage activity serves as a control. (E) Quantifications of the detection limitation of Pfu Cascade-Cas3 on dsDNA, ssDNA, ssRNA, and RNA/DNA heteroduplex. Dotted red lines indict background fluorescence. (F) Schematic for HASTE as a one-step and heat-activatable nucleic acid detection tool. (G) Excellent consistency between PCR amplification and PCR-coupled HASTE in detecting the presence of target DNA across 22 solution samples. As low as 1 attomolar target DNA was reliably detected by PCR-HASTE and no false positive was reported. (H, I) Side-by-side comparison of the collateral ssDNA cleavage activity of Pfu I-A and Tfu I-E Cascade-Cas3 in the presence or absence of a cognate DNA target, and with (I) or without (H) ATP present, respectively.

FIG. 7. I-A Pfu Cascade-Cas3 causes bi-directional deletion in RNA-guided fashion in human cells. (A) Experimental procedure for Pfu Cascade-Cas3 mediated genome editing in human cells. (B) Design of the crRNA guides targeting the template (G1) (SEQ ID NO: 52 & SEQ ID NO: 53) and non-template (G2) (SEQ ID NO: 54 & SEQ ID NO: 55) strands of the GFP ORF. (C) Quantification of the editing efficiency evaluated by FACS, based on the loss of the GFP signal. An overnight incubation at 42° C. immediately after RNP delivery increased the editing efficiency from 36.4% to 92.4%. (D) Quantification of G1 and G2 editing efficiency following different experimental procedures. (E) Bracketing PCR based detection and estimation of genome deletion around the targeting site. (F) Representative Sanger sequencing results (SEQ ID NOs: 56-59) revealing the deletion boundary, presumably formed by NHEJ-mediated DNA repair. (G) Distribution of the deletion range and size from the Sanger-sequencing results. Note that the vast majority are bi-directional deletions. Target site is eliminated as the result. (H) Mechanism-based classification of Type I CRISPR-Cas systems. I-A and possibly I-D systems use a distinct allosteric activation mechanism to degrade the substrate. These systems may be ancestral to the rest of the Type I systems because they share a stronger mechanistic similarity with Type III systems.

FIG. 8. Reconstitution and biochemical characterization of I-A Pfu Cascade-Cas3 effector complex, related to FIG. 1. (A) Co-expression scheme and schematic explanation of three strategies to promote efficient pre-crRNA procession in complex reconstitution. (B) RNA quality evaluated by denaturing-PAGE in each of the three strategies described in A. (C) Elution profile of the successfully purified Pfu Cascade on size-exclusion chromatography (SEC). (D) SDS-PAGE analysis of the SEC peak fractions. (E) Native-PAGE analysis of the SEC peak fractions stained with Coomassie blue and EtBr for protein and nucleic acid components. (F) Comparison of the SEC profiles of the reconstituted Pfu Cascade-Cas3, Cascade, Cas8a-Cas3, Cas3, and Cas8a complexes and components. (G) Representative SDS-PAGE analysis of the SEC peak fractions containing the Pfu Cascade-Cas3 complex. (H) SDS-PAGE analysis of the purified Cas8a, Cas3 (HD+HEL), Cas3 HEL, Cas HD, and Cas8a-Cas3 (HD+HEL). (I) Comparison of the SEC profiles of Pfu- and Tfu-Cascade+Cas3, Cascade, and Cas3. Pfu-Cascade complexes with Cas3 in the absence of the target DNA, whereas Tfu-Cascade fails to do so. (J) Native-agarose EMSA showing Pfu Cascade-Cas3 has much tighter affinity for the target DNA than Pfu Cascade alone. (K) Schematic diagram explaining the in vivo interference setup. (L) Evaluation of the PAM code used by Pfu Cascade-Cas3 using dilution-spotting assay. (M) Quantification of the interference efficiency mediated by three different PAMs using the assay format depicted scheme in K. (N) Native-agarose EMSA confirming that CCC PAM promotes tight binding by Pfu Cascade-Cas3, presumably through R-loop formation. FAM-labeled dsDNA is rendered in blue color, Cy3-labeled Cas3 in green, Cascade not colored. (O), (P) Native-agarose EMSA revealing that Pfu Cascade-Cas3/DNA interaction remains stable in the presence of 0.5 M NaCl (O) or at 66° C. (P), respectively. Pfu Cascade/DNA interaction is less stable at each category.

FIG. 9. Flow-chart of the cryo-EM single particle reconstructions of Pfu Cascade-Cas3 in four different functional states, related to FIG. 2. (A), (B) Workflow of the cryo-EM image processing and 3D reconstruction for the apo Pfu Cascade complex. (C), (D) Workflow of the cryo-EM image processing and 3D reconstruction for the Pfu Cascade-Cas3 complex. (E), (F), (G) Workflow of the cryo-EM image processing of the Pfu Cascade-Cas3/dsDNA complex, which produced two high-resolution 3D reconstructions, Pfu Cascade-Cas3/partial R-loop (F) and Pfu Cascade-Cas3/full R-loop (G).

FIG. 10. Structure-function analysis of Pfu Cascade and Pfu Cascade-Cas3, related to FIGS. 2 and 3. (A) Comparison of the overall shape and backbone curvature among representative I-A, I-C, I-E, and I-F Cascades. PDB accession codes are denoted. I-A Pfu Cascade resembles I-C Dvu Cascade in overall shape and curvature. (B) Tabulating the differences in the subunit stoichiometry and crRNA length among subtypes of Cascades. (C). I-A and I-C cas operon differences. I-C cas11 is encoded by a hidden ORF inside cas8c. I-A cas11 is separately encoded and cas8a further encodes a Cas11-like domain. (D) Left: Sequence and structure similarity between Cas8a CTD (SEQ ID NO: 60) (the Cas11-like domain) and Cas11 (SEQ ID NO: 61). Interface residues mediating the Cas11-Cas11 interaction are highly conserved in the Cas11-like domain. Right: comparison of the 2D topographic structure between Cas11-like domain of Cas8a and Cas11. (E) Arrangement of the Pfu Cascade “inner belly”, assembled from Cas8a and multiple copies of Cas11. Cas11 and Cas11-like domain in Cas8a are structural homologs, with a Cα r.m.s.d. of 4.35 Å. (F) I-A Pfu Cas11 is structurally homologous to the I-C Cas11c (PDB: 7KHA), I-E Cas11e (PDB: 5U07), and I-F Cas11 (Csy1 fused) (PDB: 6B44) (G) Cas8a variants knock out interference assay. P. furiosus strains with only the I-A effector module (I-A only), deletion of all effector modules (Null), or with only the I-A module and deletion of either Cas8a-1 or Cas8a-2 (ΔCas81-1 or ΔCas8a-2 respectively) were challenged with either a plasmid containing a miniature CRISPR array consisting of three spacers targeting three essential genes on the P. furiosus chromosome (Target: +) or the same plasmid lacking these spacers (Target: −). (H) Partial sequence alignment between Cas8a-1 (SEQ ID NO: 62), which assembles into Pfu Cascade and used in this structural study, and its paralog in Pfu I-A cas operon Cas8a-2 (SEQ ID NO: 63). Interface residues to Cas11 in Cas8a-1 are not conserved in Cas8a-2, which explains why Cas8a-2 does not assemble into Pfu Cascade and not functional for interference.

FIG. 11. Structure-function validation of the PAM recognition mechanism by Pfu Cascade-Cas3, related to FIG. 4. (A) SEC profiles of various Pfu Cascade samples bearing PAM-recognition residue mutations. (B) SDS-PAGE analysis of the integrity of the PAM recognition mutants purified in (A). (C) Native-agarose EMSA quantifying the extent of DNA-binding reduction caused by each PAM-recognition mutation. (D) Sequence alignment showing that the PAM-recognition residues are highly conserved among I-A Cas8a homologs (SEQ ID NOs: 65-69). (E) Same sequence alignment among Cas5a homologs (SEQ ID NOs: 70-75). Again, PAM-recognition residues are highly conserved.

FIG. 12. Thorough analysis of the RNA-guided and R-loop-dependent allosteric activation mechanism inside Pfu Cascade-Cas3, related to FIG. 5. (A) Denaturing urea-PAGE showing that the HD-nuclease activity of Pfu Cas3 was robust by itself or in complex with Cas8a. It became undetectable upon complex formation with Pfu Cascade. The assay was performed using a 5′-FAM labeled ssDNA. (B) Temperature-dependent nuclease assay analyzed on urea-PAGE revealed that the autoinhibition was robust in various temperatures. At higher temperatures and in the presence of a cognate DNA target, both the target and the ssDNA reporter were robustly degraded by Pfu Cascade-Cas3. Coloring scheme is the same as in FIG. 3C. (C) Quantification of the banding intensities in B. to reveal the nuclease activities changes in various conditions. (D), (E) Further urea-PAGE and band quantification analyses to compare the nuclease activity changes against cognate dsDNA substrate and the ssDNA reporter in the presence or absence of ATP. (F) Superposition of Cas8a before and after full R-loop formation revealing the hinge motion between Cas8a NTD and its C-terminal Cas11-like domain. (G) Superposition of Cas11.1-5 in different functional states revealing the twisting motion before and after full R-loop formation. (H) Superposition of Cas3 in different functional states revealing the global and local motions before and after full R-loop formation. (I) Workflow of the cryo-EM image processing and 3D reconstruction for the Pfu Cas3-Cas8a sub-complex. (J) The resulting 6.6 Å Pfu Cas3-Cas8a sub-complex reconstruction reveals that the HD-nuclease center adopts an open conformation, similar to the active state in the full R-loop Pfu Cascade-Case structure. This is consistent with Pfu Cas3-Cas8a possessing highly active nuclease activity.

FIG. 13. Additional data supporting the development of a nucleic acid detection platform from I-A Pfu Cascade-Cas3, related to FIG. 6. (A) Nucleic acid detection assay with different poly-deoxynucleotide ssDNA-FQ reporters. Left: fluorescence changes in the test tube. Right: quantification of the fluorescence changes over time. (B) Temperature-dependency of the collateral damage activity by Pfu Cascade-Cas3 upon encountering a cognate DNA target. The system was highly active at 45-85° C. (C) Piloting experiment to evaluate the detection limit against various nucleic acid targets. NT, Non-target; NTS, Non-target strand. (D) Schematic diagram explaining the binary reporting setup in the lateral flow strip assay. (E) The lateral strip assay reliably detected dsDNA at 100 fM concentration. (F, G) Nuclease activity changes inside I-A Pfu Cascade-Cas3 and I-E Tfu Cascade-Cas3, without or with ATP present, respectively. In the absence of ATP, Tfu Cascade-Cas3 suffered high background and small dynamic range issues. In the absence of ATP, Tfu Cascade-Cas3 suffered low sensitivity issue. Coloring scheme is the same as in FIG. 3C.

FIG. 14. Additional data supporting the bi-directional deletion activity in Pfu Cascade-Cas3, in vitro and in vivo, related to FIG. 7. (A) RNA-guided plasmid cleavage by Pfu Cascade-Cas3, at different temperatures and +/−ATP. Pfu Cascade-Cas3 mainly nicks plasmids in the absence of ATP, but switches to a processive degradation behavior in the presence of ATP. (B) Comparison of GFP knock-out experiments in HAP1-GFP and HEK291-GFP cells, in quadruplicates. (C) Real-time GFP signal monitoring after delivery of Pfu I-A Cascade-Cas3 in HEK293 and Hap1 cell lines. (D) Different concentration combination of Cascade: Cas3 for I-A system. (E) Schematics of the substrates used in the DNA degrading assay. The circular plasmid was linearized by different restriction enzymes to place PAM into different locations. (F) Deletion polarity was defined by observing the relative stability of the two cleavage product bands. Pfu I-A Cascade-Cas3 was found here to degrade both the PAM-proximal and -distal dsDNA. (G) Mapping of the Pfu I-A Cascade-Cas3 cleavage sites on TS and NTS, using a dual-labeled DNA substrate, at 37 or 42° C., +/−ATP, +/−HD active site mutation. (H) Illustration of the cleavage pattern in (G).

FIG. 15. Overview showing Type I-A Cascade and Cas3 form an integral effector complex, Type I-A Cas3 nuclease activity is allosterically activated by Cascade, upon full R-loop formation, Type I-A CRISPR-Cas3 can be repurposed into a heat-activated streamlined nucleic acid detection platform (HASTE), and that the Type I-A CRISPR-Cas3 displays highly efficient deletion-editing activity in human cells.

FIG. 16. Schematic illustrating a representative chimeric guide RNA precursor (Pre-cRNA) coding sequence array, showing upstream and downstream repeat sequences encoding E. coli and P. furiosus repeat and 5′ and 3′ handle sequences, a chimeric repeat sequence, and a spacer sequence, and E. coli Cas6 cleavage sites (SEQ ID NOs: 76-79).

FIG. 17. Related to FIG. 16, an effector complex comprising a processed chimeric guide RNA comprises PfuCas5a binding, E. coli Cas6E binding, and interaction with a double stranded DNA template comprising a PAM, and a Cas3 protein comprising Cas3′ and Cas3″ domains (SEQ ID NOs: 80-82).

FIG. 18. Photographic representation of crRNA phenol-extracted from reconstituted I-A Pfu_Cascade using chimeric guide RNA approach, related to FIGS. 16 and 17. Although Pfu_Cascade complex can form in the presence of Pfu_Cas6, the extracted crRNA from this complex is very heterogeneous due to poor pre-crRNA processing. As the result, Pfu_Cascade displayed very poor RNA-guided DNA cleavage activity (data not shown). Introducing a hammerhead ribozyme for pre-crRNA processing did not solve the problem. In contrast, when Pfu Cas components were co-expressed with E. coli Cas6 and a synthetic CRISPR operon containing engineered chimeric CRISPR repeats, the purified PfuCascade contained very pure crRNA component. In the presence of Pfu_Cas3, this heterologously assembled PfuCascade was highly active in RNA-guided DNA cleavage reactions.

FIG. 19. In vitro and in vivo data supporting the bi-directional deletion activity in Pfu Cascade-Cas3, related to FIG. 7. (Panel A) The cleavage activity increases dramatically from 37° C. to 42° C. (Panel B) Transformation after cleavage assay in (Panel B) and phenol extraction. (Panel C) Representative images to show phase and GFP channel of cell culture. (Panels D, E) Representative views of Guide 1 (Panel D), Guide 2 (Panel E) mediated GFP knock-out experiment in HAP1-GFP cells, in triplicates. (Panel F) Representative views of Guide 2 mediated GFP knock-out experiment in HAP1-GFP cells, in triplicates. (Panel F) Representative views of Guide 1 and Guide 2 combined GFP knock-out experiment in HAP1-GFP cells, in triplicates.

### DETAILED DESCRIPTION OF THE DISCLOSURE

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

All nucleotide sequences described herein include the RNA and DNA equivalents of such sequences, i.e., an RNA sequence includes its cDNA. All nucleotide sequences include their complementary sequences.

This disclosure includes every amino acid sequence described herein and all nucleotide sequences encoding the amino acid sequences, and all other polynucleotide sequences described herein. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences. For amino acid sequences of this disclosure that include amino acids that comprise purification or protein production tags, such as HIS tags, streptavidin tags, protease recognition sites, the disclosure includes the proviso that the sequences of the described tags may be excluded from the amino acid sequences. Amino acids between the described tags may also be excluded.

All temperatures and ranges of temperatures, all buffers, and other reagents, and all combinations thereof, are included in this disclosure.

All nucleotide and amino acid sequences identified by reference to a database, such as a GenBank database reference number, are incorporated herein by reference as the sequence exists on the filing date of this application or patent.

The disclosure includes all embodiments illustrated in the Figures provided with this disclosure.

Any component of the editing systems described herein can be provided on the same or different polynucleotides, such as plasmids, or a polynucleotide integrated into a chromosome. In embodiments, at least one component of the system is heterologous to the cells. In eukaryotic cells, all components of the system can be heterologous.

In embodiments the present disclosure provides compositions and methods for improving the specificity, efficiency, or other desirable properties of the described Type I-A CRISPR-based gene editing or target destruction in any eukaryotic cell or eukaryotic organism of interest. The disclosure is also suitable for use with prokaryotic organisms, such as for use as an antimicrobial system.

As used herein, the term “Cascade” refers to an RNA-protein complex that is responsible for identifying a DNA target in crRNA-dependent fashion. In this regard, Cascade (CRISPR-Associated Complex for Anti-viral Defense) is a ribonucleoprotein complex comprised of multiple protein subunits and is used naturally in bacteria as a mechanism for nucleic acid-based immune defense. Cascade complexes are characteristic of the Type I CRISPR systems. The Cascade complex recognizes nucleic acid targets via direct base-pairing to an RNA guide contained in the complex, which in this disclosure is the described chimeric RNA guide. As further elucidated in the Examples below, acceptance of target recognition by Cascade results in a conformational change which recruits a protein component referred to as Cas3. Cas3 may comprise a single protein unit which contains helicase and nuclease domains. After target validation by Cascade, Cas3 nicks the strand of DNA that is looped out by the R-loop formed by Cascade approximately 9-12 nucleotides inward from the PAM site. Cas3 then uses its helicase/nuclease activity to processively degrade substrate nucleic acids, moving in a 3′ to 5′ direction. While the specific mechanisms by which I-A Cas3 generates a bi-directional DNA deletion is still being elucidated, the biochemical activity as disclosed herein is in contrast with Type I-E and I-C CRISPR systems, which primarily introduce uni-directional deletions upstream of the PAM-side of the target site. Thus, the present disclosure demonstrates that the described systems participate in bi-directional degradation of the DNA substrate. In one embodiment, a single Cas3 effector complex generates bi-directional degradation of the DNA substrate. In another embodiment, at least two Cas3 effector complexes generates the bi-directional degradation of the DNA substrate. In embodiments, more than one round of bi-directional degradation is performed, e.g., sequential-directional degradations are performed using the described systems.

The present disclosure thus includes P. furiosus Cascade proteins, and P. furiosus Cas3, and the described derivatives thereof, and may further comprise a heterologous Cas6. Any other P. furiosus protein described herein by way of the figures and their accompanying descriptions may also be included in the systems, compositions and method of this disclosure.

As described in the Summary above, Pfu Cascade contains one copy of Cas8a, one copy of Cas5a, and multiple copies of Cas7a and Cas11a. Pfu Cas3″ and Cas3′ subunits assemble into functional Cas3 protein after expression. In embodiments, Cas3″ (referred to as “HD” or the nuclease domain) and Cas3′ (referred to as the helicase domain) are two domains of single Cas3 protein. As also described above, Pfu Cascade and Cas3 assemble at 1:1 molar ratio into an integral effector complex. In certain embodiments, an RNP of this disclosure, whether in vitro or with cells, may also include a heterologous Cas6. Thus, Cas6 can be an optional component in certain instances, such as where RNP is assembled prior to being exposed to a DNA substrate in vivo or in vitro. The Cas6 protein is an optional component of the diagnostic aspect of the described compositions, systems and methods.

Representative and non-limiting examples of Pfu protein sequences used in embodiments of this disclosure are as follows:

Pfu_Cas8a
(SEQ ID NO: 8)
MKFNEFKTPQIDPIFDLYVAYGYVESLIRGGAKEATLIPHGASYL
IQTDVSNEEFRHGLVDALSEMLSLHIALARHSPREGGKLVSDADF
SAGANINNVYWDSVPRNLEKVMKDLEKKRSVKGTATIPITLMPSA
GKYMLKHFGVQGGNPIKVDLLNYALAWVGFHYYTPYIKYAKGDTT
WIHIYQIAPVEEVDMISILSLKDLKMHLPHYYESNLDFLINRRLA
LLYHLLHSESLGALELFTEKEFVIHSYTLERSGNNQAIRSFEEEE
IGKLMDFLWKLKRRDFYHAIKFIDDLLKKATEGALALIDAIMNER
LEGFYTALKLGKKAGVVSSREIVAALEDIICER
Pfu_Cas5a
(SEQ ID NO: 9)
MDILLVCLRFPFFSVAKRSYQVRTSFLLPPPSALKGALAKGLILL
KPEKYASSSLDEAALKAIKEIESKLVDIKAVSVAPLSPLIRNAFL
LKRLRNLESGSNAEKSDAMRREYTFTRELLVAYIFKNLTQEEKNL
YLKAAMLIDVIGDTESLATPVWASFVKPEDKKAPLAFSAPYTEIY
SLLSSKIQAKGKIRMYIEKMRVSPEYSKTKGPQEEIFYLPIEERR
YKRIVYYARTIYPPEVEKALTVDGEVLGIWIPKNSSES
Pfu_Cas7a
(SEQ ID NO: 10)
MYVRISGRIRLNAHSLNAQGGGGTNYIEITKTKVTVRTENGWTVV
EVPAITGNMLKHWHFVGFVDYFKTTPYGVNLTERALRYNGTRFGQ
GETTATKANGATVQLNDEATIIKELADADVHGFLAPKTGRRRVSL
VKASFILPTEDFIKEVEGERLITAIKHNRVDVDEKGAIGSSKEGT
AQMLFSREYATGLYGFSIVLDLGLVGIPQGLPVKFEENQPRPNIV
IDPNERKARIESALKALIPMLSGYIGANLARSFPVFKVEELVAIA
SEGPIPALVHGFYEDYIEANRSIIKNARALGFNIEVFTYNVDLGE
DIEATKVSSVEELVANLVKMVGGKE
Pfu_Cas11a
(SEQ ID NO: 11)
MGGWIRNIGRYLSYLVDDTFEEYAYDVVDGIAKARTQEELLEGVY
KALRLAPKLKKKAESKGCPPPRIPSPEDIEALEEKVEQLSNPKDL
RKLAVSLALWAFASWNNCPKKGKGTEGGVE
Cas3″ (HD nuclease subunit, also
referred as Cas3 HD)
(SEQ ID NO: 12)
MSCKAFQGQTLREHIEAMLAAWEIVKNKYIPSIIRVMKTVGVKFT
EEDADKFMKTLIILHDVGKCSEVYQKHLSNNEPLRGFRHELVSAY
YAYNILKDMFKDETIAFIGALVVMMHHEPILMGQIRSLDKEELTP
EVVLDKLRTFNGVMEGTESFIKSMIKEKLGVIPKVPSPTQEDVLR
EVIRLSVLARHRPDSGKLRMVVGALLIPLVLCDYKGAKEREGESP
KFAEVLRVEMMK
Pfu_Cas3′ (helicase subunit, also
referred to as Cas3 HEL)
(SEQ ID NO: 13)
MDTEKLFRELTGFEPYDYQLRAWEKIREIMNNGGKVIIEVPTAGG
KTETAVMPFFAGIYNNNWPVARLVYVLPTRSLVEKQAERLRNLVY
KLLQLKGKSKEEAEKLARELVVVEYGLEKTHAFLGWVVVTTWDAF
LYGLAAHRTVGNRFTFPAGAIAQSLVIFDEVQMYQDESMYMPRLL
SLVVGILEEANVPLVIMSATIPSKLREMIAGDTEVITVDKNDKNK
PSRGNVKVRLVEGDITDVLNDIKKILKNGKKVLVVRNTVRKAVET
YQVLKKKLNDTLANPSDALLIHSRFTIGDRREKERALDSARLIVA
TQVVEAGLDLPNVGLVVTDIAPLDALIQRIGRCARRPGEEGEGII
LIPVENCIEHEKIVRGLSELMEKIGEDTVVFATVTSTNEYDRVVE
IHYGEGKKNFVYVGDIDTARRVLEKKRSKKLPKDLYIIPYSVSPY
DPLVLLTTYDELSKIGEYLADTTKARKALDRVYKFHYENNIVPKE
FASAYIYFKELKLFSAPPEYELRSRPELYVLLYPMNIEKNERVED
KVIDNLETARIIRISYSVKEWKKSDVVIGRLMKEWDKNAEKWVWK
VRKSFKIDPYEIYVIDAKYYNSELGFITNLSDTNSHTDSDSKVRT
RNSEHSSKKNRSKGKKGQTSLENWGVRV
A Pfu Cas6 sequence is
(SEQ ID NO: 14)
MYLSKVIIARAWSRDLYQLHQGLWHLFPNRPDAARDFLFHVEKRN
TPEGCHVLLQSAQMPVSTAVATVIKTKQVEFQLQVGVPLYFRLRA
NPIKTILDNQKRLDSKGNIKRCRVPLIKEAEQIAWLQRKLGNAAR
VEDVHPISERPQYFSGDGKSGKIQTVCFEGVLTINDAPALIDLVQ
QGIGPAKSMGCGLLSLAPL

In embodiments, the Cas6 is a heterologous Cas6, and thus is not a Pfu Cas6.

In embodiments, the heterologous Cas6 is obtained or derived from any mesophilic bacterial organism. In a non-limiting embodiment, the Cas6 is an E. coli Cas6, or a derivative or homologue thereof. The sequence of E. coli Cas6 is known, such as via the KEGG database entry b2756, from which the amino acid sequence of the Cas6 protein is incorporated herein as it exists on the effective filing date of this application or patent. A comparison of the E. coli and Pfu Cas6 proteins shows 22.21% sequence similarity across the length of the respective protein sequences. Thus, in embodiments, a heterologous Cas6 protein as used in this disclosure comprises a Cas6 protein that has less than 85.00%-22.21% sequence similarity to the sequence of a described Pfu Cas6 protein. In embodiments, the heterologous Cas6 comprises no more than 22.21%-84.99%, inclusive, and including all numbers and ranges of numbers there between to the second decimal point, sequence similarity, to the described Pfu Cas6 protein sequence.

Any protein described herein can be modified to improve its intended or actual use. In embodiments, for use in eukaryotic cells, the protein may be modified to include, for example, any suitable nuclear localization signal. In embodiments, the protein may be modified to include a linker amino acid sequence, including but not limited to a GS or glycine rich linker sequence, or a ribosomal skipping sequence, or a self-cleaving sequence, or a protein purification tag, or any combination thereof. In embodiments, two or more of the described proteins may be provided as a fusion protein.

Without intending to be bound by any particular theory, it is considered that target recognition and target degradation being separated by a conformational change validation step provides decreased off-target effects. This is because the nuclease component Cas3 is not present at the target site until after recognition has occurred. Additionally, the described Cascade has a 32 nucleotide spacer region (with 5 bases flipped out and not recognized by the crRNA) to provide 27 base pairs of recognition.

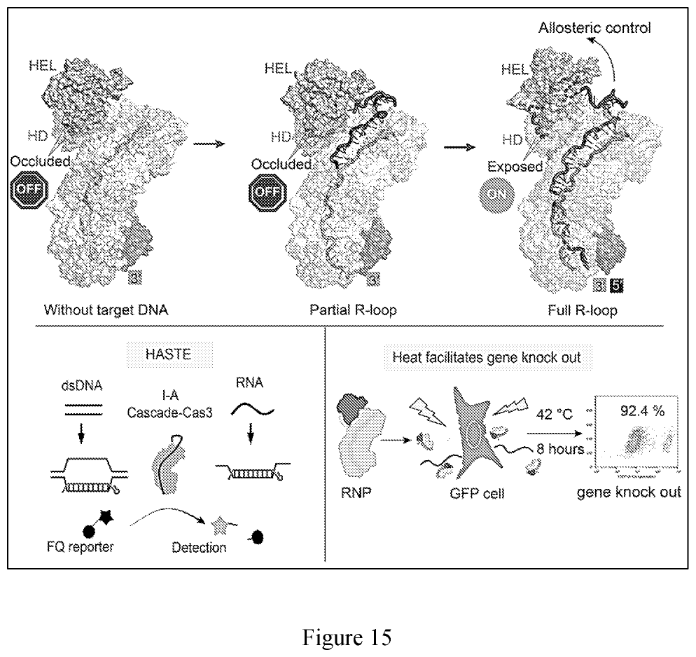

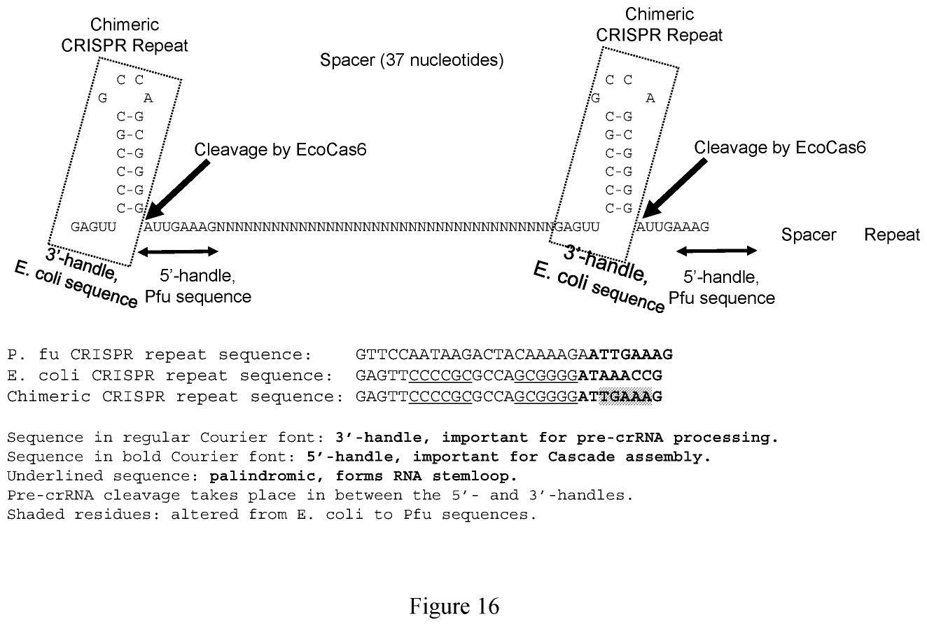

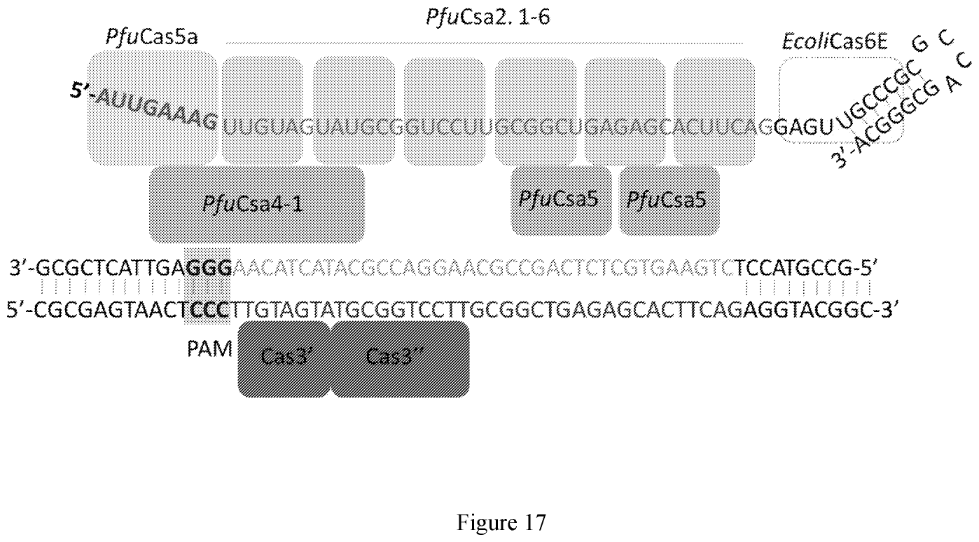

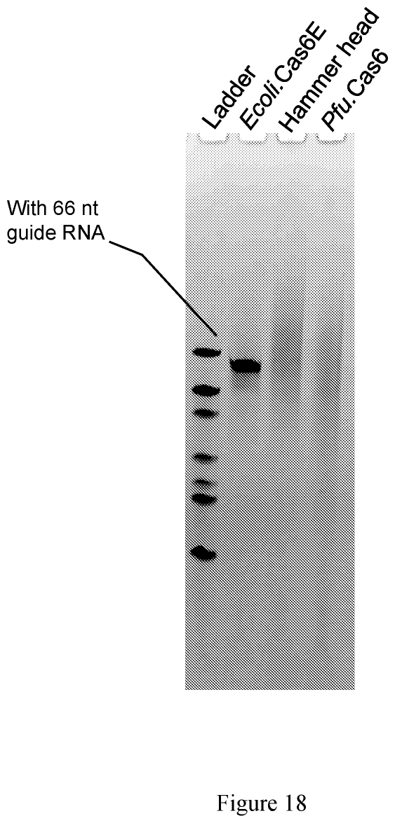

In embodiments, the disclosure comprises a crRNA as a guide RNA comprising chimeric CRISPR repeat regions at its 5′ and 3′-ends and a variable region in the middle, which comprises a spacer for RNA-guided DNA targeting, and participates in R-loop formation with the described system. The described heterologous Cas6 is involved in chimeric crRNA maturation, i.e., the described system is capable of site-specific cleavage of the chimeric CRISPR repeats in pre-crRNA in vitro and in vivo. By way of illustration, FIGS. 16, 17 and 18 provide representative illustrations of a suitable chimeric guide and its function. The top of FIG. 16 shows a chimeric guide RNA coding sequence as a partial array with flanking 3′ E. coli derived flanking sequences at each end, with the spacer and repeat orientation shown on the right side of the sequences. The 3′ handle when present in a pre-crRNA can be processed by E. coli Cas6. The chimeric guide RNA is transcribed from a synthetic array that includes a Pfu upstream repeat sequence that results in a processed guide RNA that contains a Pfu 5′ handle and a 3′ E. coli derived handle, as illustrated and annotated in the bottom portion of FIG. 15. The shaded sequence designate nucleotides changes that were introduced into the array, and convert the transcription template from an E. coli sequence to a Pfu sequence to thereby provide a template for production of a processed guide RNA that contains a Pfu 5′-handle a 3′-handle that is not encoded by a P. furiosus CRISPR array sequence, which is this case is encoded by E. coli. FIG. 17 provides a depiction of the processing of a transcript by Pfu Cas5a and E. coli Cas6, referred to as E. Coli Cas6E. FIG. 17 provides a characterization of several different test conditions that were used to process a guide RNA. In particular, FIG. 17 shows results obtained by testing crRNA phenol-extraction of in vitro reactions using reconstituted I-A Pfu_Cascade. Although the Pfu_Cascade complex can form in the presence of Pfu_Cas6, the extracted crRNA from this complex is very heterogeneous due to poor pre-crRNA processing. As a result, Pfu_Cascade displayed poor RNA-guided DNA cleavage activity (data not shown). Introducing a hammerhead ribozyme for pre-crRNA processing did not overcome this limitation. In contrast, when Pfu Cas components were co-expressed with E. coli Cas6 and a synthetic CRISPR operon, as illustrated in containing engineered chimeric CRISPR repeats, as illustrated in FIG. 15, the purified PfuCascade example contained a highly purified crRNA component. In the presence of Pfu_Cas3, this heterologously assembled PfuCascade was highly active in RNA-guided DNA cleavage reactions. Accordingly, the disclosure provides heterologous proteins and heterologous systems for producing chimeric guide RNAs that are functional with the described systems. The described chimeric guide RNAs can be produced in E. coli or other mesophilic hosts, or can be chemically synthesized, such as for use as an RNP, or in the described diagnostic assay.

In embodiments, more than one described chimeric crRNA, or chimeric guide RNA is provided. In embodiments, 2, 3, 4, 5, or more crRNAs or guide RNAs are provided.

In embodiments, any enzyme or other protein as described herein is introduced into the cell as a recombinant or purified protein, or as an RNA encoding the protein that is expressed once introduced into the cell, or as an expression vector, which is expressed once in the cell. Any suitable expression system can be used and many are commercially available for use with the instant invention, given the benefit of the present description. In embodiments, one or more components of a Cascade system described herein can be delivered to cells as an RNP, or by one or more plasmids, or a combination of proteins, RNA, and/or DNA plasmids.

In embodiments, the disclosure provides one or a combination of the following properties, relative to certain previously available approaches: i) a multi-component system exhibiting bi-directional processivity, ii) a chimeric crRNA; and iii) the system is heat-activatable.

In embodiments, a described system introduces more deletions, longer deletions, more precise deletions, or more frequent deletions in a population of cells, or a combination thereof, compared to a reference. In embodiments, the reference comprises a described system, but wherein the Cas6 is a Cas6 that is endogenous to P. Furioso, and/or wherein the chimeric guide RNA was processed using a P. Furioso Cas6. In embodiments, the reference comprises a Type I-E system.

In embodiments, the disclosure utilizes a Type I-A systems protospacer adjacent motifs (PAM) that comprises di- or tri-nucleotide conserved motifs downstream of protospacers opposite of the crRNA 5′-handle. Those skilled in the art will understand that other PAM sequences may be recognized by Cas enzymes from different bacterial types.

In embodiments, the disclosure can include a DNA molecule, such as an externally introduced DNA template, to repair the CRISPR-generated deletion, or other mutation. Thus, the disclosure includes introducing into a cell a DNA donor template, such as a single-stranded oligo DNA nucleotide (ssODN) repair template, that can yield intended nucleotide changes. Additional polynucleotides can be introduced for purposes such as creating an insertion, or a deletion of a segment of DNA in the cells. In embodiments, more than one DNA template is provided.

In embodiments, a Pfu Cascade and Cas3 or the described derivatives thereof, and optionally a heterologous Cas6, as used according to this disclosure generate one or more genome lesions, considered to be long-range deletions, wherein from the lesion(s) are initiated, or are located, from a few nucleotides from a suitable PAM sequence, and to up to 100 kb upstream of the PAM sequence, in the form of bi-directional editing.

In embodiments, the disclosure comprises one or a combination of: targeted mutagenesis by deleting one strand of DNA that is repaired by a ssDNA template via mismatch repair at the targeted site, wherein optionally the repair site is distant from the target site, wherein the distance may be up to 100,000 nts distant from the target site; recombination by engaging endogenous HDR machinery through the production of long 3′ ends which are used as homology arms during repair for insertion of a donor; processing one end of DNA into a blunt end via another nuclease; use of a DNA-binding protein to block the processivity of Cas3 activity; using a combination of Cas3 that is deleted for nuclease activity and another Cas3 that is deleted for helicase activity, and performing the method at a temperature above ambient temperature, such as at about 37° C.

The disclosure comprises the modified cells, methods of making the cells, and cells that are mutated using the compositions and methods of this disclosure, and progeny of such cells, including but not limited to modified organisms which include and/or develop from such cells.

In embodiments one or more proteins used in this disclosure has/have between 85%-100% identity to a wild type amino acid sequence, as described above. In embodiments, the protein comprises a truncation and/or deletion such that only a segment of the protein that is required to achieve a desired effect (i.e., an improvement in DNA editing/deletion relative to a reference) is achieved. In embodiments, a protein used herein comprises an amino acid sequence that includes additional amino acids at the N- or C-terminus, relative to a wild type sequence. In embodiments, proteins have 90, 91, 92, 93, 94, 95, 96, 97, 98, or 99% identity across the entire length or a functional segment thereof of the sequences described herein. Thus, derivatives of the proteins and their nucleotide sequences are included. The term “derivative” and its various grammatical forms as used herein refers to a nucleotide sequence or an amino acid sequence with substantial identity to a reference nucleotide sequence or reference amino acid sequence, respectively. The differences in the sequences may be the result of changes, either naturally or by design, in sequence or structure. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

In embodiments, the disclosure comprises use of one or more P. furiosus proteins, or one or more proteins having at from 80-99% similarity to a T. fusca protein, with the proviso that the Cas6 protein that is not encoded by P. furiosus.

In addition to the advantages of the presently provided systems described above, the present disclosure provides data demonstrating that the P. furiosus-based systems can work at physiological temperatures characteristic of mammalian, and particularly human, body temperature. Thus, in embodiments, the disclosure provides for use of the systems described herein comprising P. furiosus protein(s) or derivatives thereof, and optionally a heterologous Cas6, wherein modifying DNA in eukaryotic cells is performed at a temperature that is higher than ambient temperature, ambient temperature being typically about 30° C. In embodiments, the disclosure provides for using the described systems at a temperature of about 37° C., although data presented herein shows the described systems can work at higher temperatures. In embodiments, performing a method of the disclosure at a temperature of about 37° C. results in improved function, relative to performing the method of the at such a temperature with a system that does not include the described proteins. The term “about” 37° C. means the temperature may be from 36.0-38.0° C. In embodiments, the improved function comprises any one or a combination of the functions described in Table A.

As discussed above, in embodiments, the disclosure includes a chimeric crRNA, which may be considered a “targeting RNA”. A crRNA, when transcribed from the portion of the CRISPR system encoding it, comprises at least a segment of RNA sequence that is identical to (with the exception of replacing T for U in the case of RNA) or complementary to (and thus “targets”) a DNA sequence in a cell into which the system is introduced. In embodiments the targeting RNA is complementary to a sequence in a chromosome in a eukaryotic cell, or to a dsDNA extrachromosomal element, such as a dsDNA viral genome. Thus, the disclosure includes modifying chromosomes, and dsDNA extrachromosomal elements. The type of dsDNA extrachromosomal elements are not particularly limited. The dsDNA extrachromosomal element may be linear, or circular. In an embodiment, the extrachromosomal element is a viral dsDNA, and/or a cytoplasmic dsDNA that may or may not be from a virus. In embodiments, the extrachromosomal element contains segments of genomic sequences, i.e., segments of one or more chromosomes are present in the extrachromosomal element.

The sequence of the targeting RNA is not particularly limited, other than by the requirement for it to be directed to (i.e., having a segment that is the same as or complementarity to) a CRISPR site that is specific for a target in the cell(s) wherein a modification is to be made, and that it can function in a Cascade complex described herein, or as will otherwise be apparent to those skilled in the art. Non-limiting embodiments of DNA that comprises a targeted sequence are provided. In embodiments, using a system described herein, the PAM and the protospacer sequence (the target sequence) is not modified. In embodiments, crRNA for a system according to this disclosure, such as a P. furiosus system, is typically 66 nt long. The crRNA has 37 nt spacer with an 8 nt and 21 nt 5′ and 3′ handle sequence at each end, respectively. Thus, in embodiments, the 5′ handle and the 3′ handle may comprise or consist of 8 nucleotides, and 21 nucleotides, respectively.

In embodiments, the modification of genetic content in a cell using Type I-A CRISPR system described herein is improved relative to a reference. Improvement of the modification can include but is not necessarily limited to improved length of a deletion, or the amount of cells in which DNA modification takes place. Thus, in embodiments, the present disclosure provides for introducing a described Cascade system into a population of cells, wherein the DNA is modified in from 10%-100% of the cells in the population. In embodiments, between 1,000 to between one and three million cells are present in the population. In embodiments, between about 100,000 to about 300,000 cells are present in the population. In embodiments, at least 100,000 cells are present in the population. The amount, number, percentage, etc., of cells in which the DNA modification takes place can be determined using routine approaches, such as by DNA sequencing of the cells in the population.

In embodiments, the disclosure comprises deleting a segment of a chromosome. The deletion may be single or double stranded. In embodiments, the deletions comprise from 500 base pairs, to 100 K base pairs, inclusive, and including all ranges of numbers there between, and including base pair deletions, and wherein the deletions may be achieved in a bi-directional manner.

In embodiments the disclosure comprises modifying a cell or a population of cells, such as eukaryotic cells by introducing into the cells one or a combination of expression vectors or other polynucleotides encoding a Cascade system.

In embodiments the disclosure may further comprise introducing into cells a DNA mutation template that is intended to be fully or partially inserted into a chromosome or other genetic element within a cell via operation of the present improved Type I-A CRISPR-Cas system. In embodiments the DNA mutation template comprises a DNA sequence that is homologous to a selected locus in a designated chromosome, and thus may be incorporated into a target genetic element via cooperation of the Type I CRISPR system and any type of homologous recombination. In embodiments the DNA mutation template can comprise a DNA segment having any nucleotide length and homology with a host cell genetic segment comprising a selected locus, so long as the length and sequence identity are adequate to introduce the intended genetic change into the locus via functioning of the Type I CRISPR-Cas system described herein. In embodiments, the DNA mutation template is a single-stranded oligo DNA nucleotide (ssODN). In embodiments, the DNA mutation template is a double-stranded (ds) template. In embodiments, the DNA mutation template is provided as an extrachromosomal element, such as a plasmid or PCR product. The DNA mutation template in certain aspects comprises a segment to be inserted into a chromosome. The segment can be inserted into a protein-coding or non-protein coding portion of a chromosome, or may be present in a regulatory control element, including but not necessarily limited to a promoter or enhancer element, a splice junction, etc.

In embodiments, the cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made. In embodiments, the cells are human, or are non-human animal cells. In embodiments, the cells are mammalian cells. In one approach the cells are engineered to express a detectable or selectable marker or a combination thereof.

The DNA sequence that is targeted is not particularly limited, other than a requirement for it to be linked to the sequence that is to be degraded. As such, the chimeric guide RNA does not need to target a specific mutation, but only target a sequence that is linked to the mutation. By “linked” it is meant that the sequence to which the guide RNA is directed is on the same chromosome or extrachromosomal element to be degraded. In embodiments, an RNA coding sequence is targeted. In embodiments, an intron is targeted. In embodiments, a non-coding, non-intronic sequence is targeted. In embodiments, an essential gene is targeted, such that the modification of the essential gene may be lethal to the cell. In embodiments, more than one DNA sequence is targeted, such as by using multiple Cascade systems concurrently or sequentially, and/or by introducing more than one distinct chimeric guide RNA.

In embodiments, the described systems, compositions and methods are used to degrade a target in a chromosome that comprises a mutation. The mutation may be specific to a certain cell type. In embodiments, the mutation is specific to cancer cells, or a viral sequence. In certain examples, the mutation is a dominant negative mutation. In embodiments, the mutation causes a loss of heterozygosity. In embodiments, the mutation activates an oncogene. In an embodiment, the mutation results in derepression of a gene.

In embodiments, the mutation comprises a trinucleotide repeat (TNR), such as in a DNA element that has undergone or is undergoing TNR expansion. In embodiments, the mutation activates a kinase. In embodiments, the mutation targets a KRas mutation, representative examples of which include KRAS (G12C), KRAS (V12V) and KRAS (G21D).

The sequences of KRAS mutations are well known in the art. The location of the KRAS sequence that is mutated may be on human chromosome 12, such as at chr12: 25398281-25398303. In non-limiting embodiments, the disclosure includes use of a chimeric guide RNA that targets the KRAS (G12V) mutation. In an embodiment, the chimeric guide RNA comprises the sequence AUUGAAAGACAGCUCCAACUACCACAAGUUUAUAUUCAGUCAUUUGAGUUCCC (GCGCCAGGGGGG (SEQ ID NO: 15) wherein the bold nucleotides comprise a Pfu 5′ handle, the italicized nucleotides comprise a an E. coli 3′ handle, the A at position 9 is a single nucleotide difference between the wild type and the mutated sequence, and the intervening sequence comprises a spacer sequence.

In an embodiment, the disclosure includes use of a chimeric guide RNA that targets the KRAS (G12D) mutation. In an embodiment, the chimeric guide RNA comprises the sequence AUUGAAAGUCAGCUCCAACUACCACAAGUUUAUAUUCAGUCAUUUGAGUUCCC CGCGCCAGGGGGG (SEQ ID NO: 16) wherein the enlarged nucleotides comprise a Pfu 5′ handle, the italicized nucleotides comprise a an E. coli 3′ handle, the U at position 9 is a single nucleotide difference between the wild type and the mutated sequence, and the intervening sequence comprises a spacer sequence.

In embodiments, the disclosure comprises bi-directional deletion of a strand of DNA that is linked to any marker, such as a described KRAS mutation, or a SNP, triplet repeats that are associated with certain disorders, including but not necessarily limited to Myotonic dystrophy, Huntington disease, spinocerebellar ataxia, Friedreich ataxia, and fragile X syndrome.

In embodiments, the disclosure comprises bi-directional deletion of a strand of DNA that is linked to a point mutation and/or indel that affect open reading frames, i.e., exons, or splice junctions. In embodiments, bi-directional deletion of a strand of DNA occurs on a segment of the DNA that is linked to a single exon skipping splice mutation (a type I splicing mutation), or cryptic exon inclusion (a type II mutation) or an exonic mutation that affects splicing (type III and V mutations). Disorders associated with the described splicing mutations are known in the art, and the disclosure includes elimination of such mutations using the described systems. In embodiments, bi-directional deletion of a strand of DNA occurs on a segment of the DNA that comprises a 5′ untranslated region, or a 3′ untranslated region.

In embodiments, the DNA that is deleted is linked to an inherited disease associated gene. In embodiments, the DNA that is deleted is linked to an integrated viral sequence, including but not necessarily limited to an integrated retroviral sequence. In embodiments, the DNA that is deleted is linked to a short or long interspersed retrotransposable element, i.e., a SINE or a LINE. In embodiments, the DNA that is deleted is linked to a gene that is associated with an autoimmune disease. In embodiments, the DNA that is deleted is linked to a gene that is associated with a muscle disorder, including but necessarily limited to any form of muscular dystrophy. Any of the foregoing embodiments may result in inactivation of a disease causing gene.

In embodiments, the disclosure includes obtaining cells from an individual, modifying the cells ex vivo using a Type I-A CRISPR system as described herein, and reintroducing the cells or their progeny into the individual for prophylaxis and/or therapy of a condition, disease or disorder, or to treat an injury, trauma or anatomical defect. In embodiments, the cells modified ex vivo as described herein are used autologously. In embodiments, the cells are provided as cell lines. In embodiments, the cells are engineered to produce a protein or other compound, and the cells themselves or the protein or compound they produce is used for prophylactic or therapeutic applications.

In various embodiments, the modification introduced into cells according to this disclosure is a homozygous dominant or homozygous recessive or heterozygous dominant or heterozygous recessive mutation correlated with a phenotype or condition, and is thus useful for modeling such phenotype or condition. In embodiments a modification causes a malignant cell to revert to a non-malignant phenotype.

In embodiments, kits for making genetic modifications as described herein are provided. A kit comprises one or more suitable vectors that encode Type I-A Cascade proteins. The kits can also include other components that are suitable for using the expression vectors to edit DNA in any cell type.

The chimeric guide RNAs can be complexed with Cascade proteins either at the same time as or at a separate time from the production of either the guide RNAs or the Cascade proteins. The guide RNA-containing Cascade Complexes can be either produced in a cell using DNA or RNA encoding for the protein and/or RNA components or delivered in the form of one or more vectors for expression or delivered in the form of RNA encoding for the proteins and/or RNA components or delivered in the form of fully-formed protein-RNA complexes through mechanisms including but not limited to electroporation, injection, or transfection. The chimeric guide RNA-containing Cascade complexes described herein, can be recombinantly expressed and purified through known purification technologies and methods either as whole Cascade complexes or as individual proteins. These proteins can be used in various delivery mechanisms including but not limited to electroporation, injection, or transfection for whole-protein delivery to eukaryotic organisms or can be used for in-vitro applications for sequence targeting of nucleic acid substrates or modification of substrates. Cascade complexes containing guides which target a DNA sequence of interest will hybridize to the target sequence and will, if complementarity is sufficient, open a full R-loop along the length of the target site. This Cascade-marked R-loop region adopts a conformation which allows Cas3 to bind to a site which is PAM-proximal, orienting the nuclease domain to initially attack the non-targeted DNA strand approximately 9-12 nucleotides inside the R-looped region. The helicase domain is loaded with the non-target strand, and the Cas3 then processively unwinds the substrate DNA in an ATP-dependent fashion from 3′ to 5′. In conjunction with this helicase activity, nuclease activity cleaves the non-target strand in a processive and bi-directional fashion.

In embodiments, the disclosure optionally uses at least two wild-type Cas3 proteins, or modifications or derivatives thereof. For example, in a case where either wild-type Cas3 or an otherwise engineered Cas3 is capable of cleaving both strands of DNA during a processive mode, once recruited to a validated target sequence by Cascade, Cas3 inherently produces a 3′ overhang on the target strand. This is because Cascade is protecting the target strand from just after the PAM site to the end of the R-loop. Thus, once Cas3 is loaded on the non-target strand and begins its processive cleavage, the earliest nucleotide on the target strand that is available for cleavage is at the PAM site. In comparison, degradation of the non-target strand occurs 9-12 nucleotides inside the R-loop region. This introduced lesion can then be repaired with a provided donor nucleic acid template which is either single-stranded or double-stranded. The lesion can also be repaired in the absence of a donor template and due to the processive nature of Cas3 and multiple cleavage events introduced, drop-out of genomic DNA or a cross-over event can occur resulting in either production of a region deletion or in the production of a homozygous set of alleles which previously was heterozygous.

In a case where either wild-type Cas3 or an otherwise engineered Cas3 is capable of confining its cleavage activity to one or the other strand of substrate DNA, two or more Cascade targeting complexes can be used, such that the PAM sites are facing towards one another, to recruit Cas3 to each target site and degrade the intervening section of DNA on both strands. This will produce 3′ overhangs on both strands of DNA and a degraded segment of DNA between.

In a case where either wild-type Cas3 or an otherwise engineered Cas3 is capable of confining its cleavage activity to one or the other strand of substrate DNA, an approach for replacing a strand of DNA with a donor ssDNA oligo can provide a means for targeted, precise, and predictable point mutation which is PAM independent.

In a case where helicase activity of Cas3 is decreased or destroyed, one or more Cascade targeting complexes can be used to recruit Cas3 to each target site and nick the non-target strand at each site. These nicks may be recognized by DNA repair proteins in the cell and repaired with a provided DNA donor which is either single-stranded or double-stranded.

As discussed above, variations on Cascade are encompassed in this disclosure. For example, Cascade complexes may be generated to contain a DNA guide instead of an RNA guide. Temperature-sensitive mutations may also be useful to either decrease the thermal requirement of activity for a thermophilic complex or to increase the thermal tolerance of a complex. These mutations could affect protein stability, R-loop formation efficiency, expression or purification, off-target effects, or other complex functions or properties. Mutations to decrease the thermal dependence of P. Furioso Cascade R-loop formation have been performed and analyzed in previous work. Epitopes, tags, and/or functional groups may be added to Cascade to aid in visualization, localization, or to confer new activity or other properties to the Cascade complex. It may be possible to generate hybrid Cascade complexes in which subunits from different organisms are used to form a single Cascade complex which may provide distinct advantages of individual sub-units from different organisms. It may be possible to engineer an interface on Cascade such that it interacts with the Cas3 protein of a different organism or with an engineered Cas3 protein.

The disclosure also includes using Cas3 variants and derivatives. For example, mutations can be made that affect protein stability, R-loop recruitment efficiency, initial nicking efficiency, helicase activity, processive nuclease activity, expression or purification, off-target effects, or other protein functions or properties. Temperature-sensitive mutations may also be useful to either decrease the thermal requirement of activity for a thermophilic protein or to increase the thermal tolerance of a mesophilic protein. Epitopes, tags, and/or functional groups may be added to Cas3 to aid in visualization, localization, or to confer new activity or other properties to Cas3. It may be possible to engineer an interface on Cas3 such that it interacts with the Cascade complex of a different organism or with an engineered Cascade complex.

Cascade complexes containing guides which target a nucleic acid sequence of interest can be tagged through protein fusion to any number of fluorescent proteins or groups for chemical modification and addition of fluorescent groups or some other functional unit that allows for detection, or by fusion to an antigen that allows for detection. The crRNA that is complexed with the Cascade protein may also be chemically modified to possess a chemical group that exhibits fluorescence or another method of detection. Additionally, Cas3 in either the wild-type, nuclease dead, helicase dead, or other mutant form or any combination thereof may be fused to any number of fluorescent proteins or groups for chemical modification and addition of fluorescent groups or some other functional unit that allows for detection, or by fusion to an antigen that allows for detection. Cas3 being tagged in such a way is expected to provide lower background detection signal when visualized optically due to Cas3 only being recruited to the site of a fully-formed Cascade R-loop constituting a properly recognized and validated target sequence. Epitopes, tags, or chemical groups added to Cascade, Cas3, or a crRNA can also be used as a mechanism for affinity purification. Hybridization of the crRNA to a target sequence prior to purification allows for a pull-down of sequences with significant complementarity to the crRNA and may be used to detect a sequence of interest or to infer the copy number of a sequence of interest through a method such as quantitative PCR.

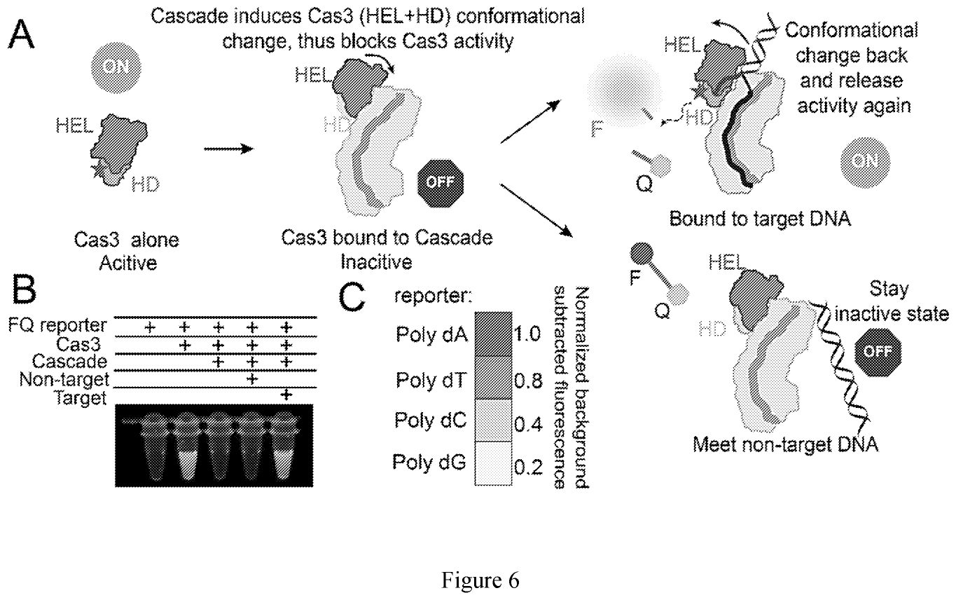

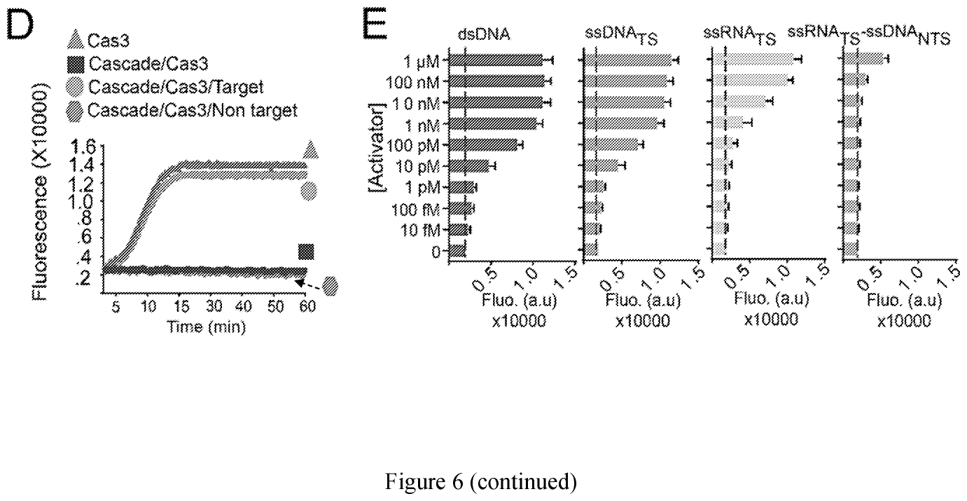

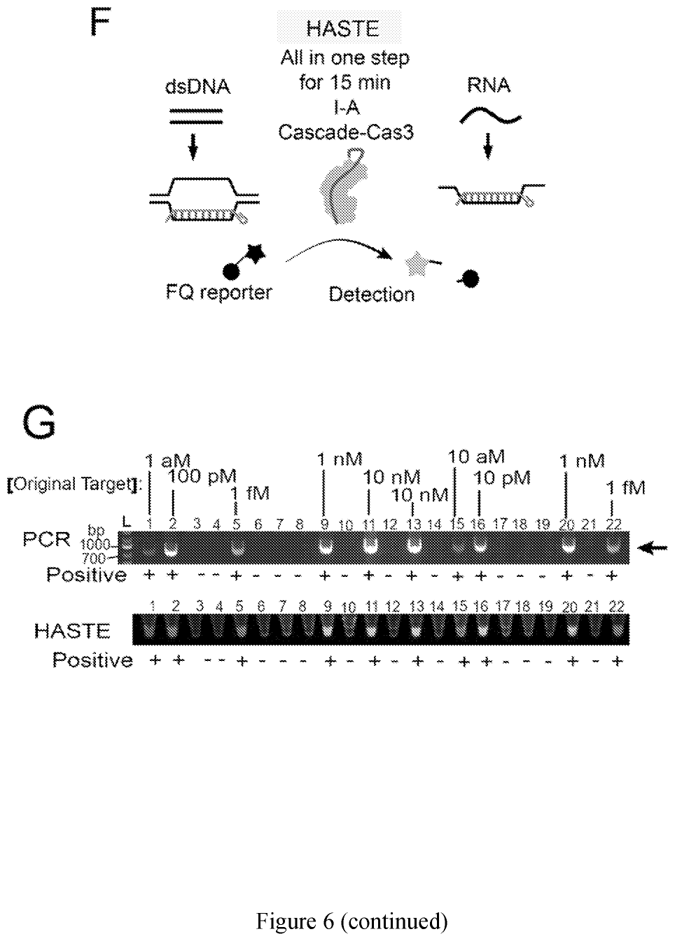

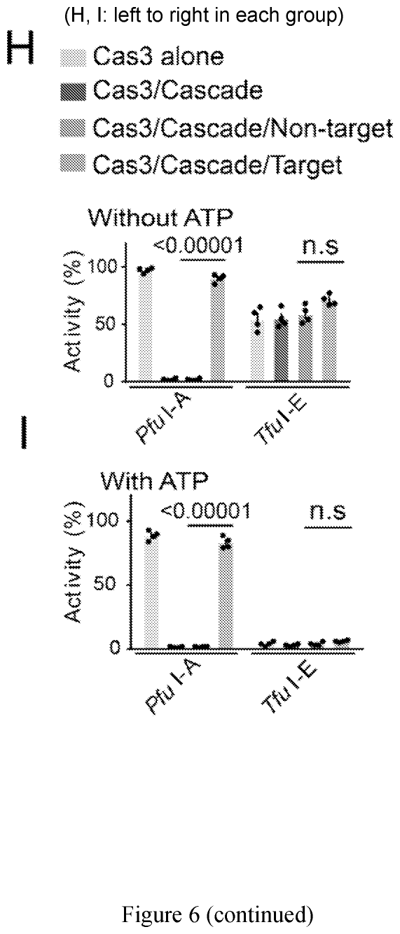

In another aspect the disclosure provides the described systems for use in an in vitro assay, which may be a comprise a cell free composition. The assay includes components of the described system, a chimeric guide RNA, and a reporter DNA construct. The composition is used in a heat-activated streamlined nucleic acid detection platform, referred to herein as HASTE. Representative embodiments of the HASTE assay are shown in the panels of FIG. 6. The assay may be performed in a single reaction tube, or may be performed in, for example, a lateral flow assay using any suitable materials, including but not necessarily limited to modified strips. One or more strips can be used, and can include control and test lanes. The controls may be positive and/or negative controls.

In a non-limiting embodiment, the described Pfu system or derivative thereof, which also comprises the heterologous Cas6, is used in an adaptation of a nucleic acid diagnostic assay known in the art as SHERLOCK (for Specific High Sensitivity Enzymatic Reporter UnLOCKing) assay, described in PCT publication WO2017219027, published Dec. 21, 2017), the disclosure of which is incorporated herein by reference.

The SHERLOCK assay is used to detect and/or quantify a target RNA or DNA or using a CRISPR Cas related approach. In the known SHERLOCK assay, a detectably labeled non-target RNA is used to provide a means of diagnostic readout using Cas13 in guide RNA programmed recognition of, for example, a polynucleotide target. Embodiments of this disclosure substitute Cas13 with the described Pfu system or derivative thereof, and include at least PfuCascade-Cas3, and the heterologous Cas6, and provide improvements over previously available assay. In performance of the assay, if a target polynucleotide is present in a sample, the described system complexes with the target RNA in the sample in a chimeric guide RNA directed manner, and non-specific nuclease activity (e.g., collateral nuclease activity) results in enzymatic degradation of a detectably labeled DNA substrate (e.g., a reporter single stranded DNA) that, for example, comprises a detectable label and a quencher. For instance, the detectably labeled ssDNA may comprise a fluorophore and a quencher moiety conjugated to the reporter DNA in sufficient proximity to one another such that the detectable signal is quenched when the reporter DNA is intact. Accordingly, when and if the DNA reporter is cleaved by the non-specific nuclease activity of the described system, which is considered to only become active once the described system has engaged a target in a chimeric guide-RNA directed manner and with application of heat, the detectable label is liberated from the intact reporter DNA, and a signal from it can be detected using any suitable approach. The system is suitable for detecting very low amounts of target in a sample, such as a little as a 1 attomolar concentration of a target. The system is suitable for detecting the presence, absence, or determining an amount of any target polynucleotide, include dsDNA, ssDNA, and ssRNA. Thus, the system can detect polynucleotides from a wide array of sources, including but not limited to prokaryotic cells, eukaryotic cells, and a variety of pathogens, such as virulent bacteria and viruses. The assay can be performed in a single reaction chamber is as little as 15 minutes. The assay can be activated or its activity increased by application of heat in the range of 37° C. to 85° C. In embodiments, any detectable label can be used with the reporter ssDNA, non-limiting examples of which include fluorophores, metals or chemiluminescent moieties, fluorescent particles, quantum dots, etc., provided the signal from the detectable label can be quenched, or its intensity shifted to a different wavelength in, for example, Förster or fluorescence resonance energy transfer (FRET).

In embodiments, the detectable signal that can be dequenched in the described HASTE assay comprises a fluorescent signal. In embodiments, a fluorophore is conjugated to 5′ or 3′ end of a reporter single stranded DNA, and a quencher molecule is conjugated to the other end (5′ or 3′ end, respectively), although one or both of these moieties could be conjugated to an internal nucleotide, provided the detectable label and quencher are in sufficient proximity such that the signal is quenched when the ssDNA is intact. In embodiments, the single stranded DNA is configured so that the quencher and detectable label are at 5′ and 3′ ends, respectively, or vice versa. Thus, by degrading the ssDNA, alleviation of quencher-mediated suppression of the fluorescence is achieved.

In embodiments, any detectable label can be used, non-limiting examples of which include metals or chemiluminescent moieties, fluorescence particles, quantum dots, and other detectable labels, provided the detectable label can be quenched by a suitable quencher. In embodiments, the detectable signal when dequenched has a wavelength of 430-520 nm, 480-580 nm, 550-650 nm, 620-730 nm, or 550-750 nm. In non-limiting embodiments, the detectable label or quencher are selected from 6-carboxyfloroscein (FAM), Cy3, Cy5, 6-Carboxytetramethylrhodamine (TAMRA), and Courmin. Other detectable label/quencher pairs are known in the art and can be adapted, when given the benefit of this disclosure, for use in the described assay.

The fluorescent or any other described signal may be interpreted using any suitable device. In embodiments, any suitable imager located proximal to an analyzed sample can be used. In embodiments, free-space optics may be used to detect a signal from the described assay using any suitable signal detection device that is placed in proximity to the location where a detectable signal is generated, such as a CCD camera. In embodiments, the disclosure provides a device for use in sample analysis. In embodiments, the device may comprise, among other features, an optical waveguide to transmit a signal to any suitable measuring device such that optical accessibility to sample is not necessarily required to detect the signal. In embodiments, lens-less optics, and/or a cell phone based imaging approach is used. In embodiments, signal analysis is performed using a device that can be connected to or in communication with a digital processor and/or a computer running software to interpret the presence, absence, and/or intensity of a signal. The processor may run software and/or implement an algorithm to interpret an optically detectable signal, and generate a machine and/or user readable output. In an embodiment, an assay device used to perform the described analysis can be integrated or otherwise inserted into an adapter that comprises a detection device, such as a camera, or a microscope, including but not limited to a fluorescent microscope. In embodiments, a computer readable storage medium can be a component of an assay device of this disclosure, and can be used during or subsequent to performing any assay or one or more steps of any assay described herein. In embodiments the computer storage medium is a non-transitory medium, and thus can exclude signals, carrier waves, and other transitory signals.

Kits comprising the described components, such as at least PfuCascade-Cas3, and a labeled ssDNA reporter, are provided.

### EXAMPLES

The following examples are presented to illustrate the present disclosure. They are not intended to be limiting in any manner.

### Example 1

Pfu Cascade-Cas3 forms an integral Type I-A effector complex.

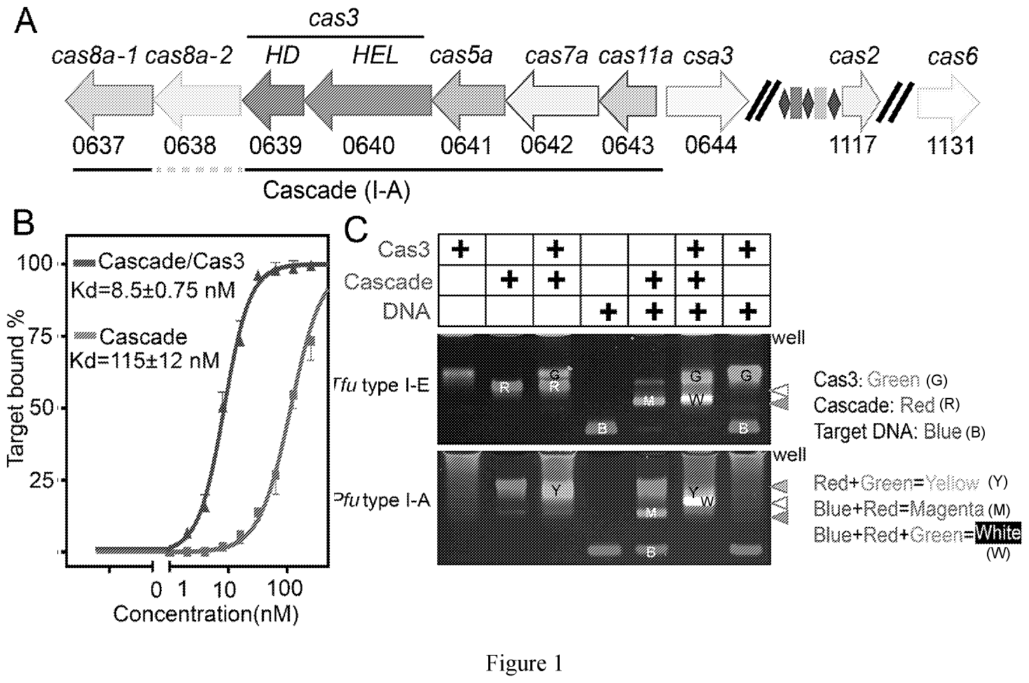

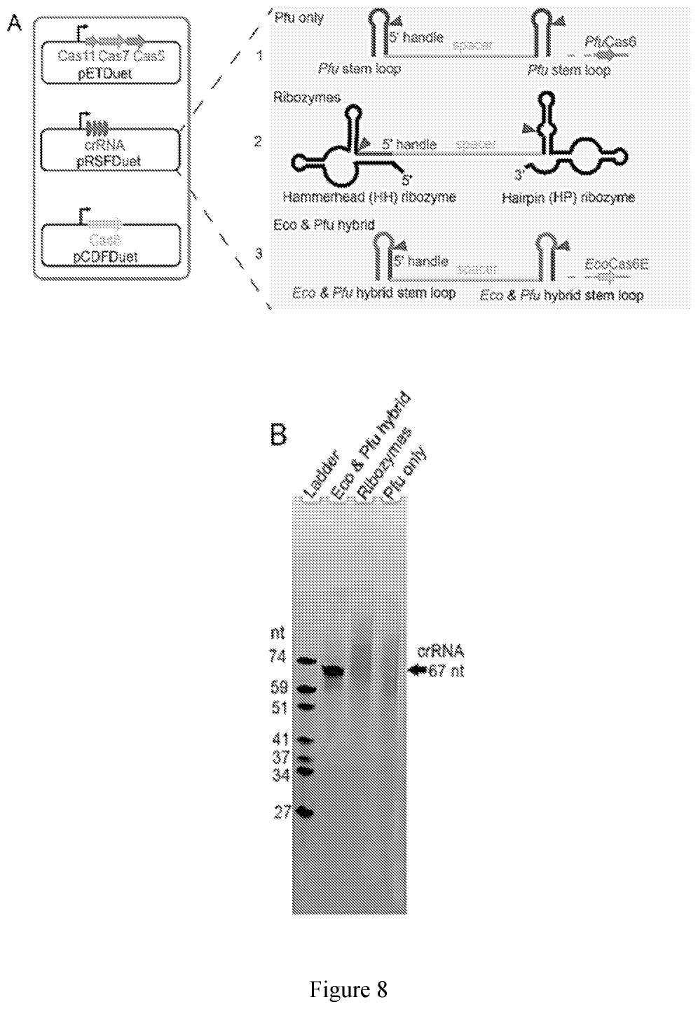

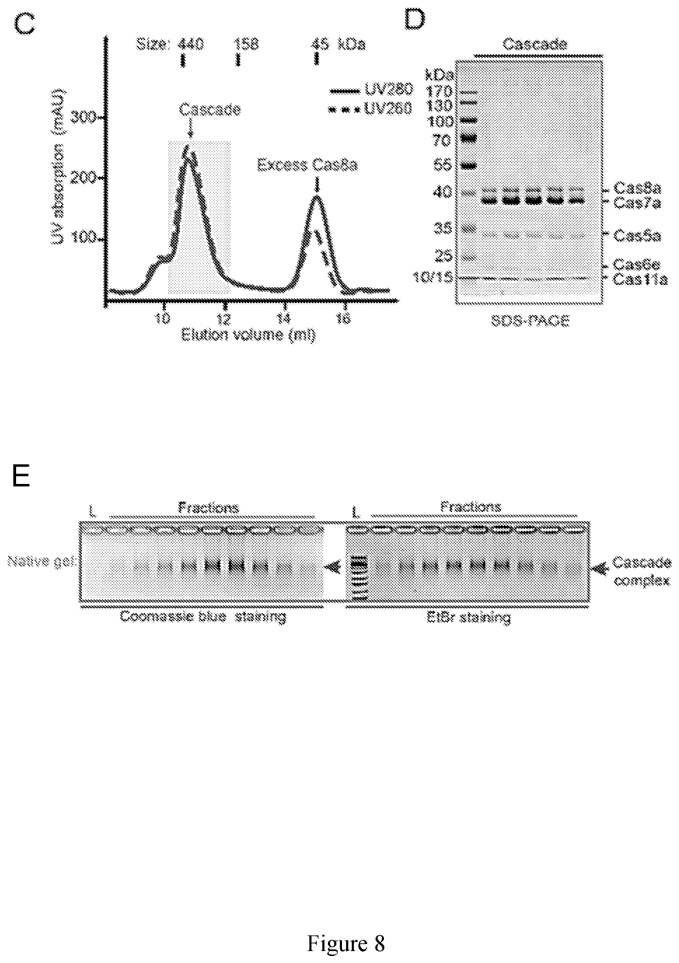

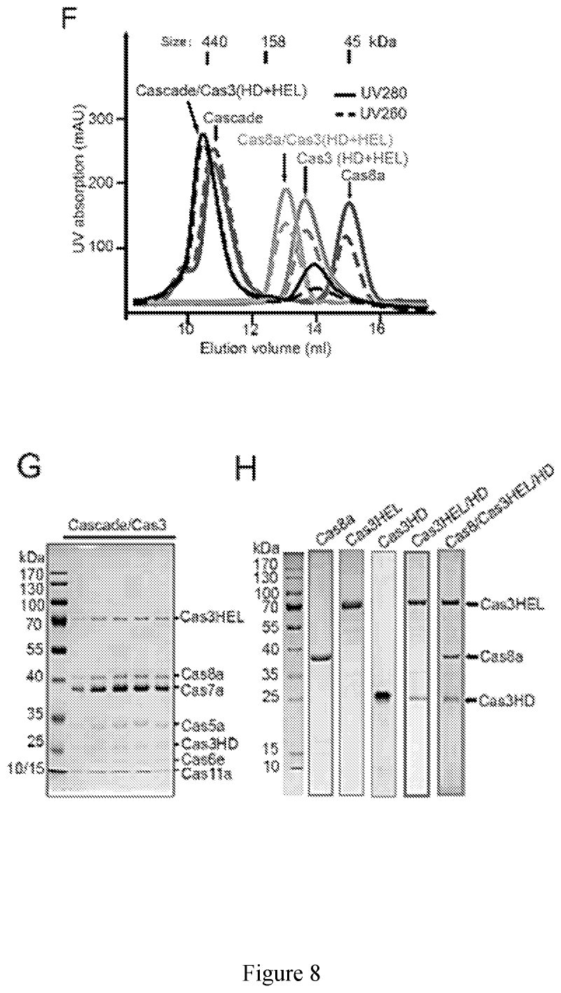

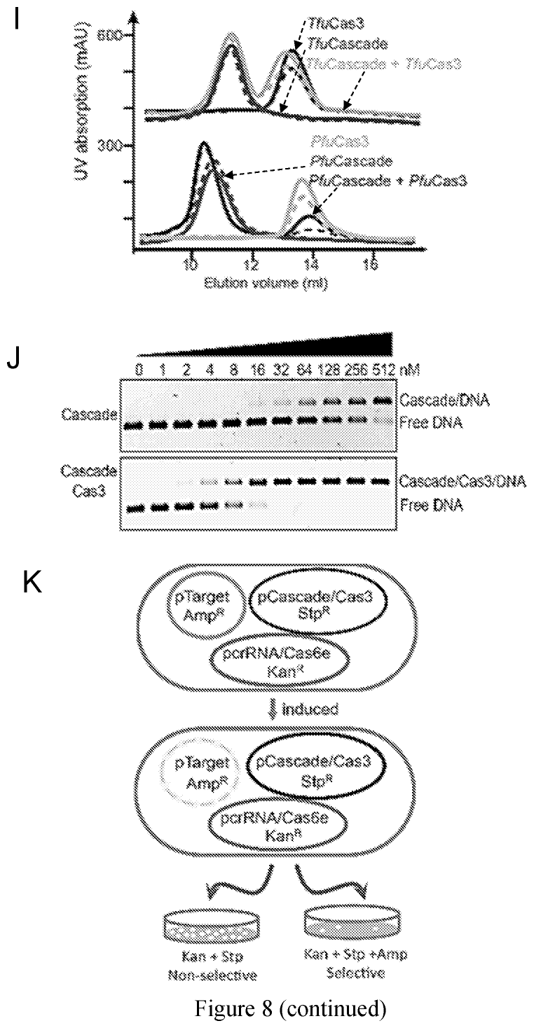

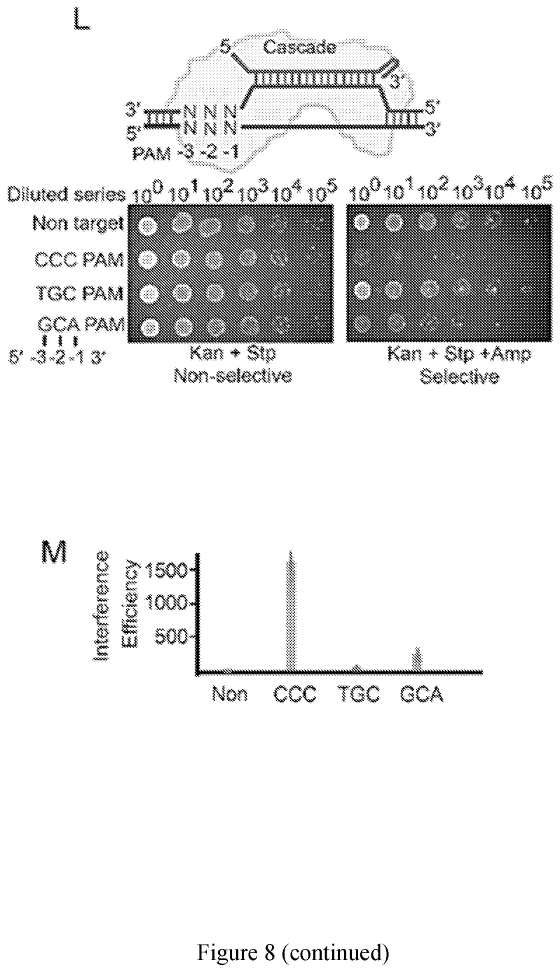

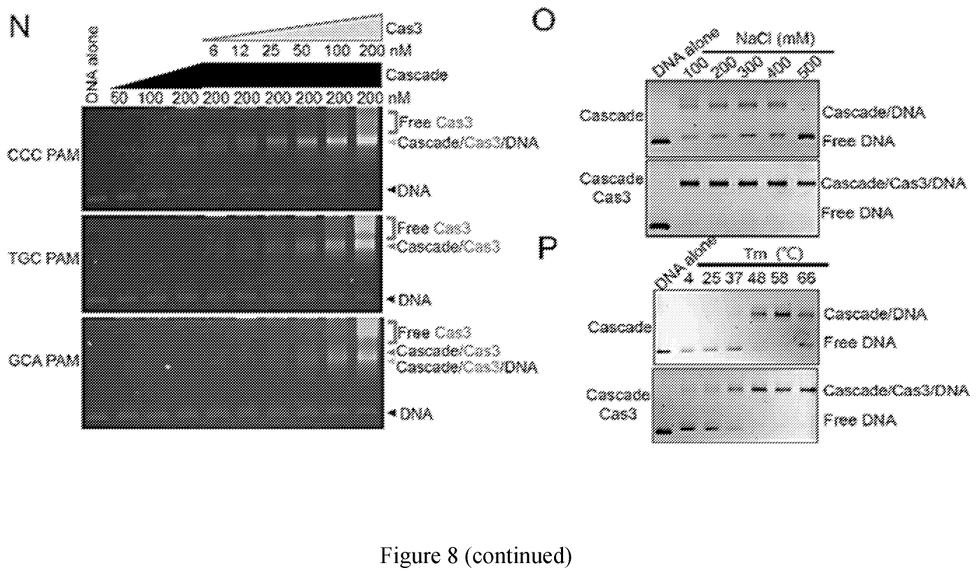

The Pfu Type I-A effector was previous reconstituted by mixing purified protein components with the mature crRNA (Majumdar and Terns, 2019). To obtain sufficient materials for the described structural studies, we used a recombinant expression approach from E. coli (FIG. 8). Initial attempts produced Cascade-like complexes that contained various-sized RNAs, possibly from unspecific incorporation of host RNAs. We speculated that this was due to the inefficient processing of pre-crRNA, since Cas6 from this extremophilic archaea has been shown to cleave the pre-crRNA poorly at ambient temperatures (Carte et al., 2008). Alternative pre-crRNA cleavage methods were tested. This included the use of self-cleaving ribozymes and the use of E. coli Cas6 for in vivo pre-crRNA processing (FIG. 8A-E). The latter strategy made use of a chimeric CRISPR repeat containing the Pfu 5′-handle and the E. coli 3′-handle that was efficiently cleaved by Eco Cas6. This approach was successful, producing high-quality Pfu Cascade with homogeneous chimeric crRNA (FIG. 8B-E). Cas3 in I-A systems is encoded by two separate open-reading frames, Cas3″ encodes the HD nuclease and Cas3′ encodes the superfamily II helicase subunit (FIG. 1A). They are renamed in this disclosure to Cas3 HD and Cas3 HEL, respectively. We show that these two subunits assemble into a stable Cas3 complex upon mixing, suggesting that they function as a unit (FIGS. 8F-H) (Majumdar and Terns, 2019).

A surprising observation was that Pfu Cascade alone interacted with the target DNA poorly, with an apparent Kd of ˜115 nM from the electrophoretic mobility shift assays (EMSAs) (FIG. 1B; 8J). Further inclusion of Pfu Cas3 improved its target-searching behavior. The Kd of Pfu Cascade-Cas3 for the cognate DNA target was 8.5 nM, comparable to that of Cascades in other subtypes (FIG. 1B). This observation differs from findings with the I-C, I-E and I-F systems, where Cascade was shown to bind the DNA target efficiently and independently, and Cas3 is recruited in a second step for DNA degradation (Govindarajan et al., 2020; Hochstrasser et al., 2014; Hochstrasser et al., 2016; O'Brien et al., 2020; Rollins et al., 2019; Xiao et al., 2018; Xiao et al., 2017). Further investigation revealed that the interaction between Pfu Cas3 and Pfu Cascade was constitutive rather than DNA substrate-dependent (FIG. 1C), and that Pfu Cas3 recognized and stably bound to its binding partner, the Pfu Cas8a subunit, alone or in the context of the intact Pfu Cascade (FIG. 8F-H). For Tfu I-E counterparts, the equivalent Cas3-Cas8 interaction was only stable in the context of the Cascade complex and after DNA binding and R-loop formation (FIG. 1C; S1I) (Hochstrasser et al., 2014; Xiao et al., 2017). Collectively, our biochemical evidence suggests that Pfu Cascade and Pfu Cas3 functions as a unit by forming an integral I-A interference complex.

A thorough round of characterization was carried out for Pfu Cascade-Cas3. RNA-guided DNA cleavage was shown to be robust and PAM-dependent in vivo and in vitro (FIGS. 8J-N). Furthermore, Pfu Cascade-Cas3 was less salt sensitive, more thermostable, and maintained DNA-binding function better at higher temperatures than Pfu Cascade alone, again consistent with Cascade and Cas3 functioning as an integral unit (FIG. 8, O-P).

### Example 2

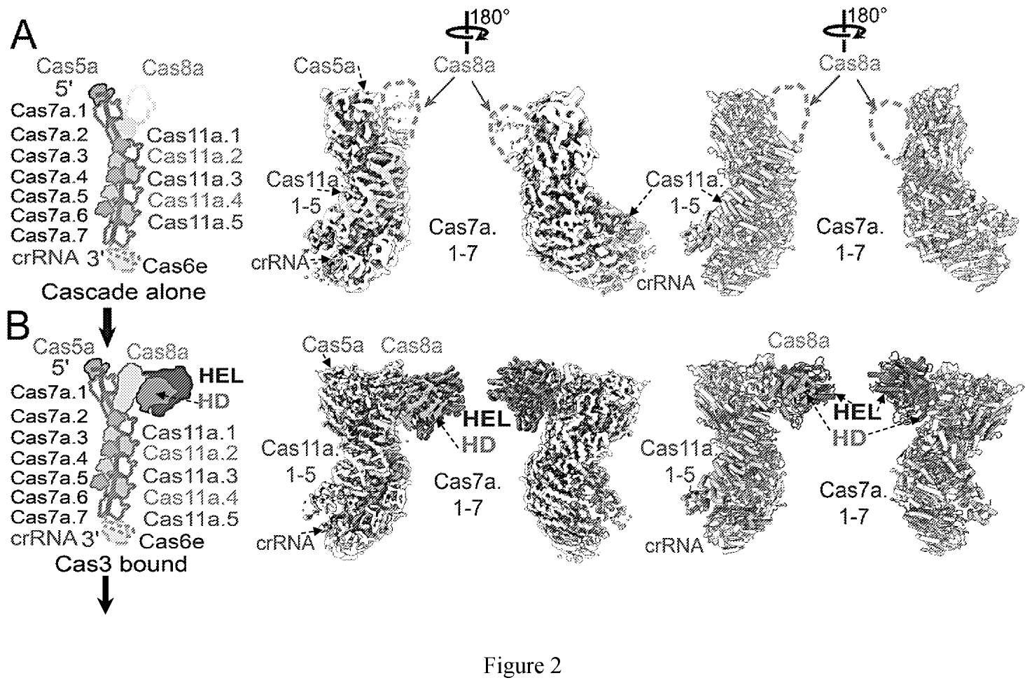

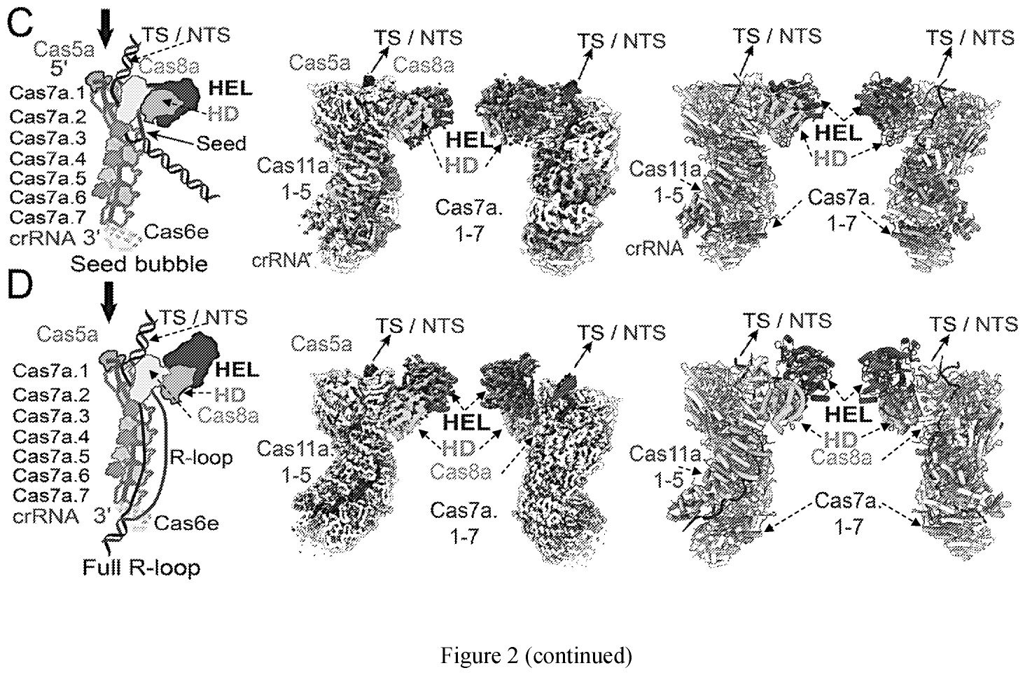

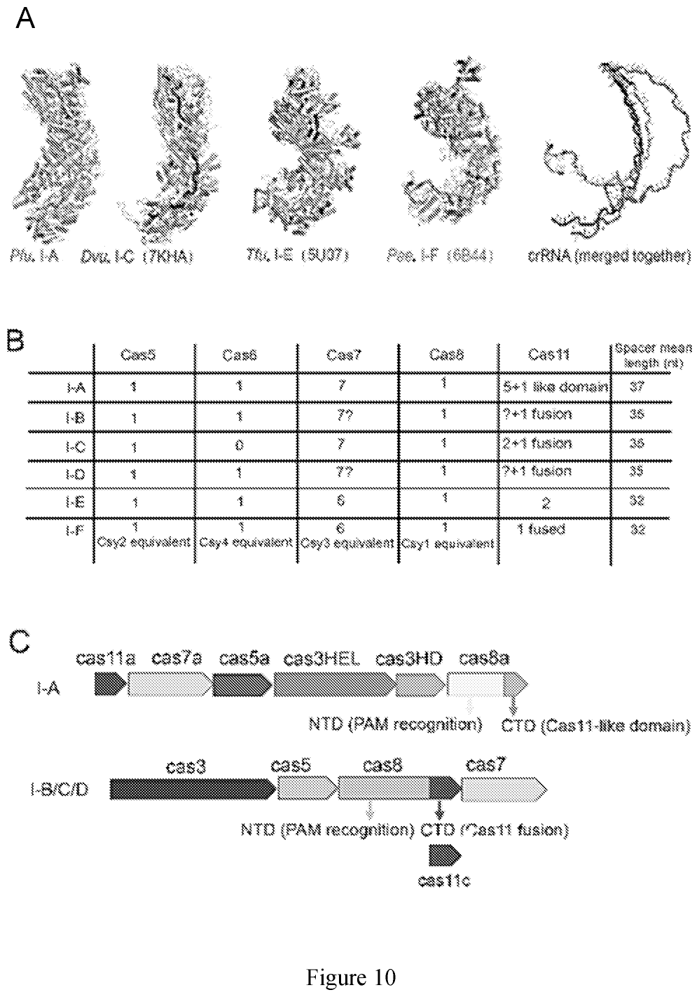

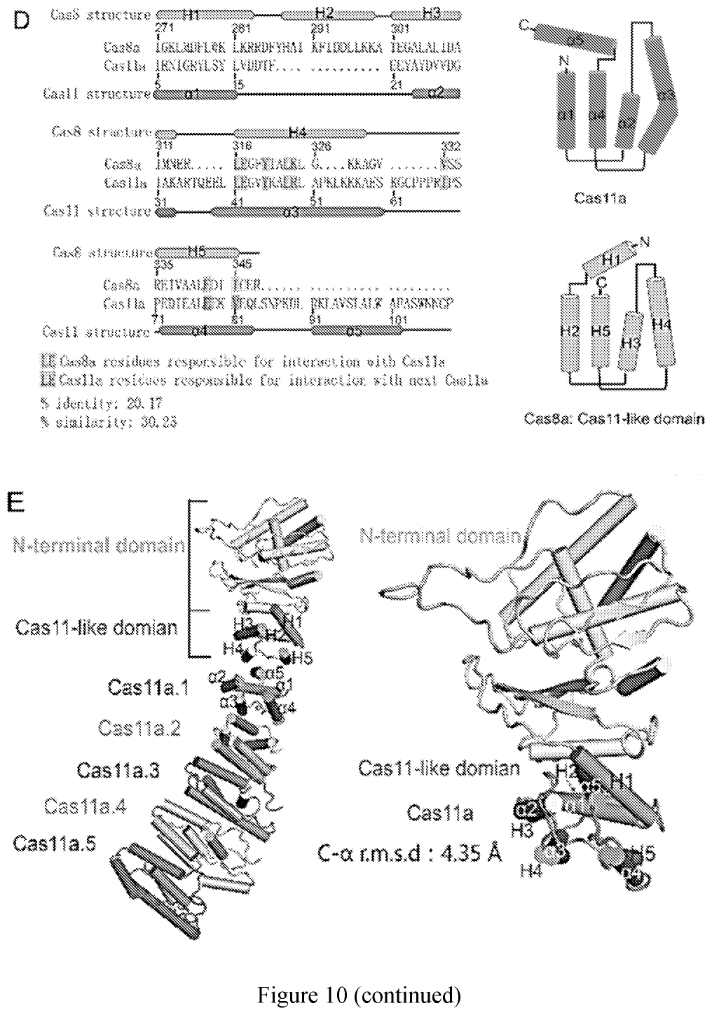

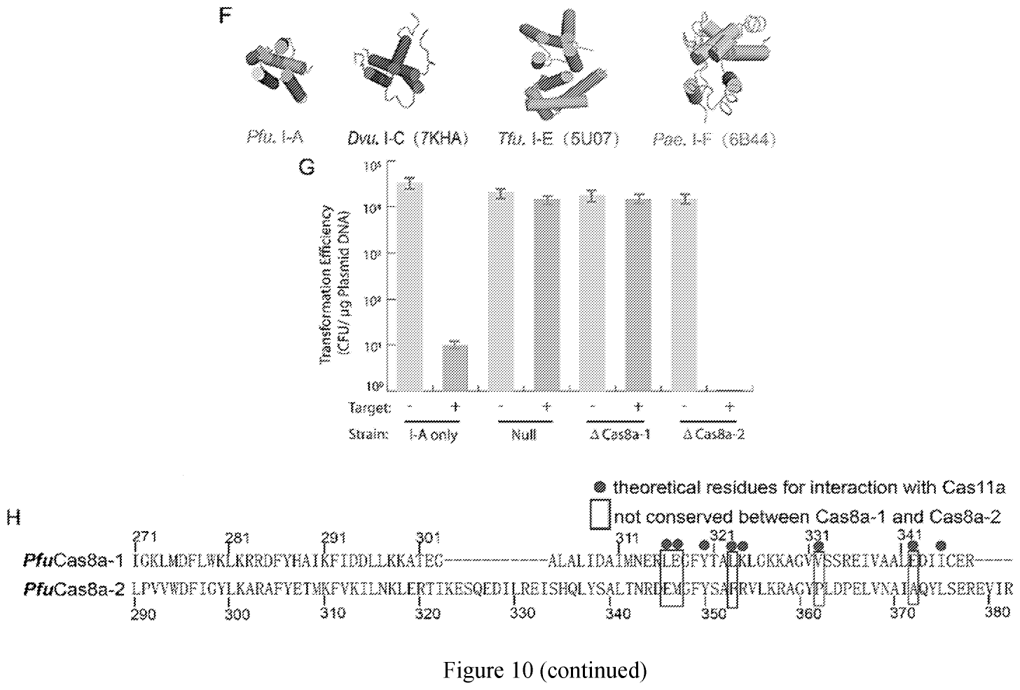

Overview of the four functional states in PfuCascade-Cas3 captured by cryo-EM. A series of four cryo-EM structures of Pfu Cascade and Cas3 were determined, each depicting a distinct functional state (FIGS. 2; 9A-G). The structures of Pfu Cascade with and without Cas3 bound were determined at 3.56 and 3.37 Å resolutions, respectively (FIGS. 2A-B; S2A-D). The structural comparison explains why Pfu Cas3 is necessary for efficient target searching by Pfu Cascade. The overall shape and composition of Pfu Cascade agrees better with the I-C Cascade than with the I-E and I-F counterparts. Both contain seven rather than six copies of Cas7 subunit in the backbone due to the longer spacer length in the crRNA in I-A and I-C subtypes (FIGS. 2A-B; 10A-B). Significant differences exist, though. For example, the Cas11 subunit found in I-B/I-C/I-D subtypes is encoded as a hidden ORF overlapping the region of the cas8 gene encoding the Cas8 C-terminal domain (CTD) (McBride et al., 2020; O'Brien et al., 2020). In contrast, the Type I-A cas operon encodes a separate cas11 ORF, which is highly homologous to cas8a CTD in sequence and structure (30% sequence similarity, 4.35 Å in Ca r.m.s.d) (FIG. 10C-E). An identical set of residues are found in Cas11 and Cas8a CTD to mediate the intermolecular interactions (FIG. 10D-F), therefore Cas8a CTD is a Cas11-like domain. Interestingly, the Pfu Type I-A cas operon encodes two versions of Cas8a (FIG. 1A). Only Cas8a-1 assembled into Cascade; Cas8a-2 led to poor reconstitution (data not shown). Thus, in embodiments, the compositions, methods and systems of this disclosure may exclude Cas8a-2. In vivo knock out of Cas8a variants also indicated that only Cas8a-1 deletion caused interference deficiency in P. furiosus (FIG. 10G). These two evidences suggest that Cas8a-1 is the functional component for the interference in the variants. This can be rationalized based on the sequence alignment, as Cas8a-2 lacks the key Cas11-interacting residues found in Cas8a-1 (FIG. 10H). The function of Cas8a-2 remains unknown.

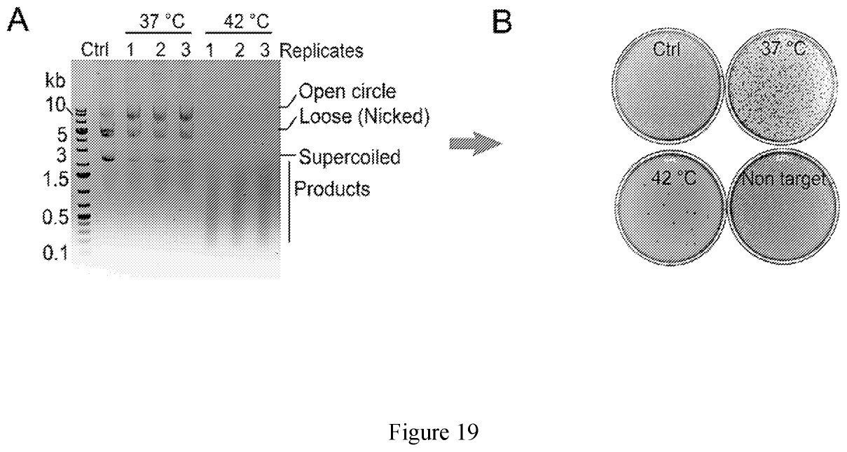

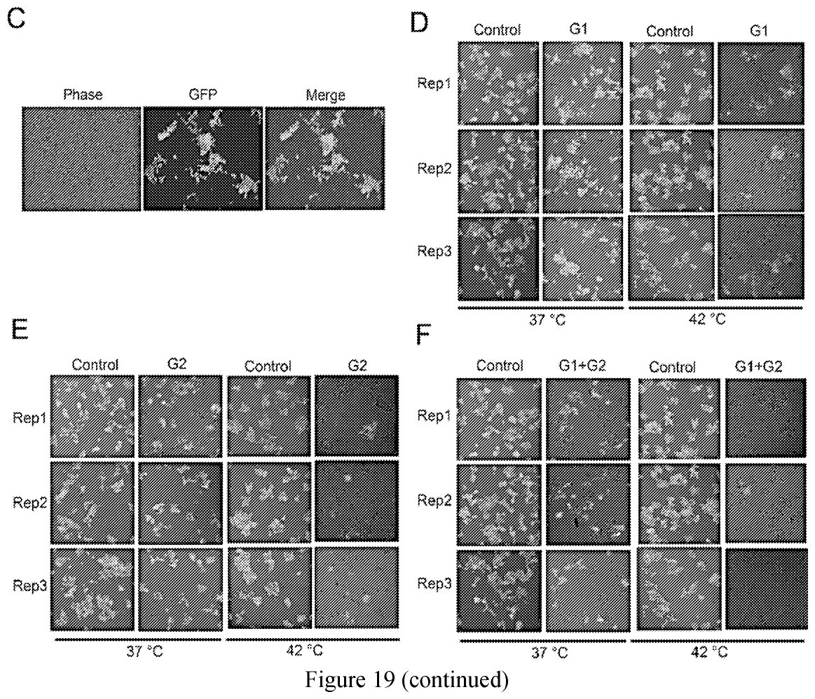

We further programmed Pfu Cascade-Cas3 with a protospacer-containing dsDNA and reconstructed two structures from the cryo-EM sample. A 3.88 Å R-loop intermediate was captured, in which the PAM-proximal 17 bp dsDNA is unwound, the 17 nt target-strand (TS) base-pairs with the complementary crRNA spacer, the first 4 nt non-target strand (NTS) can be traced, whereas the rest of NTS and PAM-distal dsDNA are not resolved in the cryo-EM density (FIGS. 2C; 9E-F). This R-loop intermediate is one-segment (6 bp) longer that the counterpart captured from the Tfu I-E Cascade/DNA sample (Xiao et al., 2017). A 3.29 Å structure of Pfu Cascade-Cas3 opening a full R-loop structure was also captured. The entire 37 nt TS DNA base-pairs with the crRNA spacer. The NTS has been nicked by Cas3 and the PAM-proximal portion re-threaded through Cas3 HEL, ready for processive degradation at Cas3 HD (FIGS. 2D; 9G, FIG. 19). A total of 13 nt can be traced for the PAM-proximal NTS strand; an additional 2-4 nt are inferred based on distance to connect the three segments of ssDNA (FIG. 2D). This is consistent with the observation that Pfu Cas3 nicks the NTS strand 15-17 nt below PAM (Majumdar and Terns, 2019). The PAM-distal NTS and dsDNA remain unresolved in the map. This is believed to be the first snapshot of a Type I effector complex poised for processive degradation of NTS DNA. The two functional states share common features, such as the PAM recognition mechanism. Significant differences exist. For example, structural comparison revealed a concerted set of conformational changes taking place in Pfu Cascade-Cas3 during the partial to full R-loop transition. This observation reveals the target-dependent nuclease activation mechanism

### Example 3

### Cas3 Primes Cascade for DNA Target-Searching

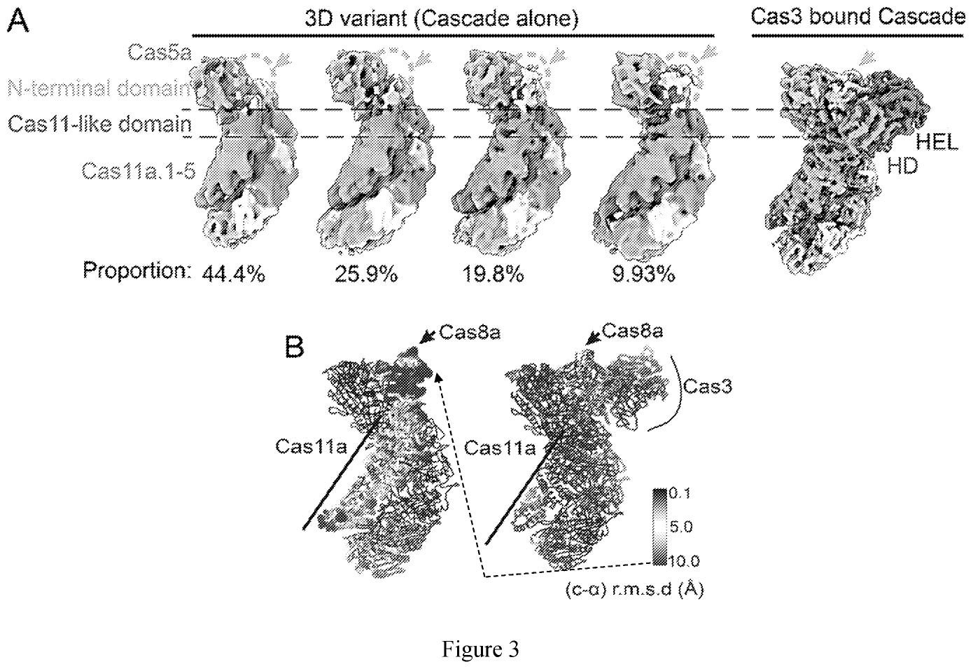

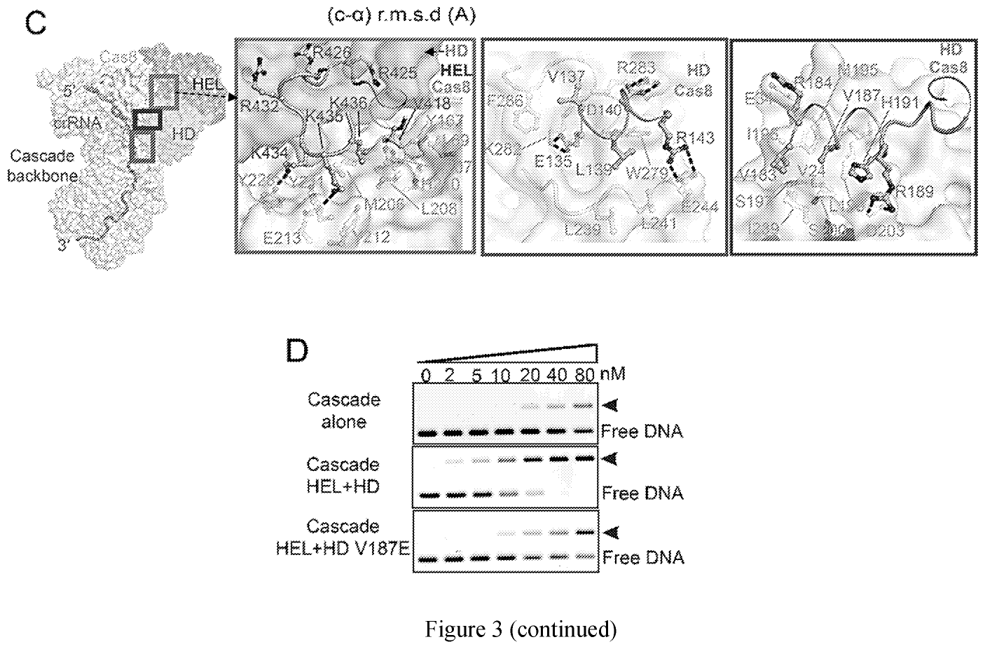

An important structural observation is that the N-terminal domain (NTD) of Cas8a in the apo Pfu Cascade is highly mobile and largely absent from the EM density. As shown in 3D classifications, less than 10% of the apo Pfu Cascade particles contain significant Cas8a NTD densities. Even in this class, the densities are not of sufficient quality to allow unambiguous docking of Cas8a NTD (FIG. 3A). The Cas8 NTD is responsible for PAM-recognition in other studied Cascades (Hayes et al., 2016). It further works with neighboring subunits to unwind the dsDNA underneath PAM, initiating R-loop formation (Xiao et al., 2017). Failing to adopt a defined conformation is expected to impede the function of Cas8a NTD to couple PAM recognition with DNA unwinding. This rationalizes the poor target-searching behavior in apo Pfu Cascade. Cas8a NTD is further connected to the Cas11-like Cas8a CTD and Cas11.1-5, together they form the so-called “inner belly” of the Cascade (Xiao et al., 2017). In apo Pfu Cascade, the entire inner belly has elevated conformational dynamics than the backbone subunits (Cas5a-Cas7.1-7), judged from the significantly reduced local map resolution (FIG. 3B).

Binding of Pfu Cas3 effectively eliminates the severe domain movements in Cas8a NTD, and rigidifies the rest of the inner belly subunits in place (FIG. 3A-B). The lockdown effect is achieved through an extensive Cas3-Cas8a interface that spans across the NTD and CTD of Cas8a, which prevents the hinge motion in between the two domains. Structural analysis revealed extensive hydrophobic and polar contacts, and the interface residues are highly conserved among Type I-A homologs (FIG. 3C; FIG. 19). To validate structural observations, we mutagenized three conserved interface residues on Cas3 individually and in combination, two from the HD nuclease (V187E, L139A) and one from the helicase subunit (V418A). In Strep-tag pull-down experiments, each mutation reduced the affinity between Cas3 and Cascade, albeit to various extents. In particular, Cas3 HD V187E and HD L139A/HEL V418A completely disrupted Cas3 interaction with Cascade (FIG. 19). Thus, in embodiments, any Cas3 sequence that is at least 85% similar to a Pfu Cas3 may retain the described wild type amino acids. Consistent with the demonstration that Cas3 binding enables target-searching by I-A Cascade, when Cas3 V187E instead of WT Cas3 was introduced to the EMSA assay, the DNA binding behavior of Pfu Cascade did not improve (FIG. 3D). Overall, our structural and biochemical data suggests that the I-A Cascade is only functional in the context of the Cascade-Cas3 effector complex.

### Example 4

### PAM Recognition

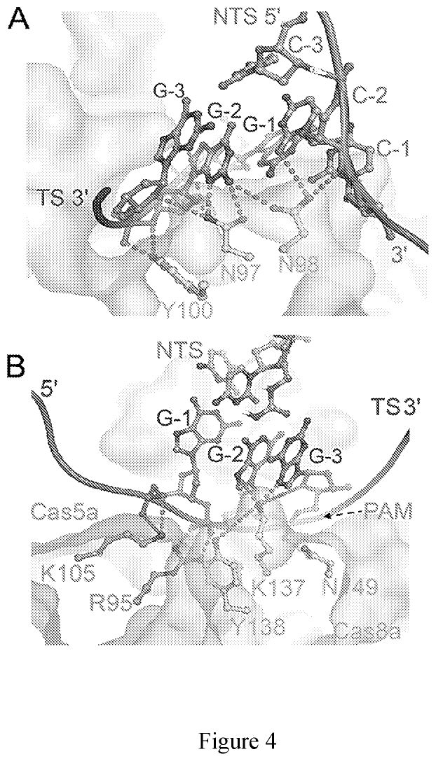

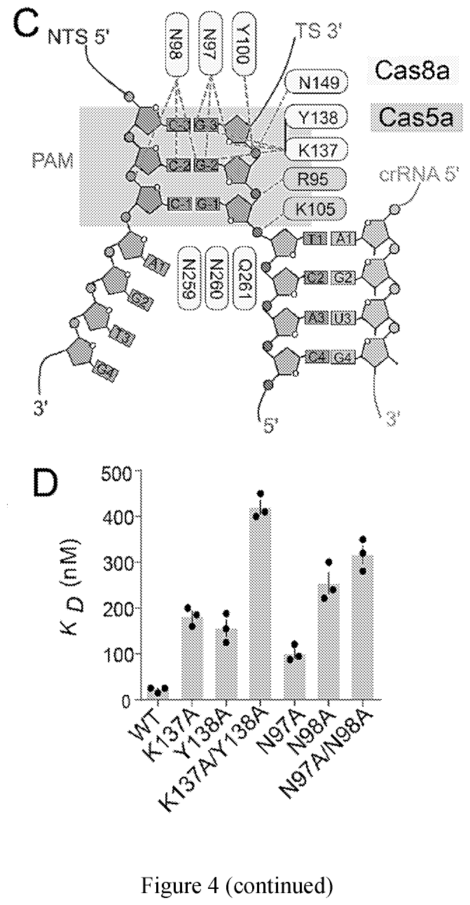

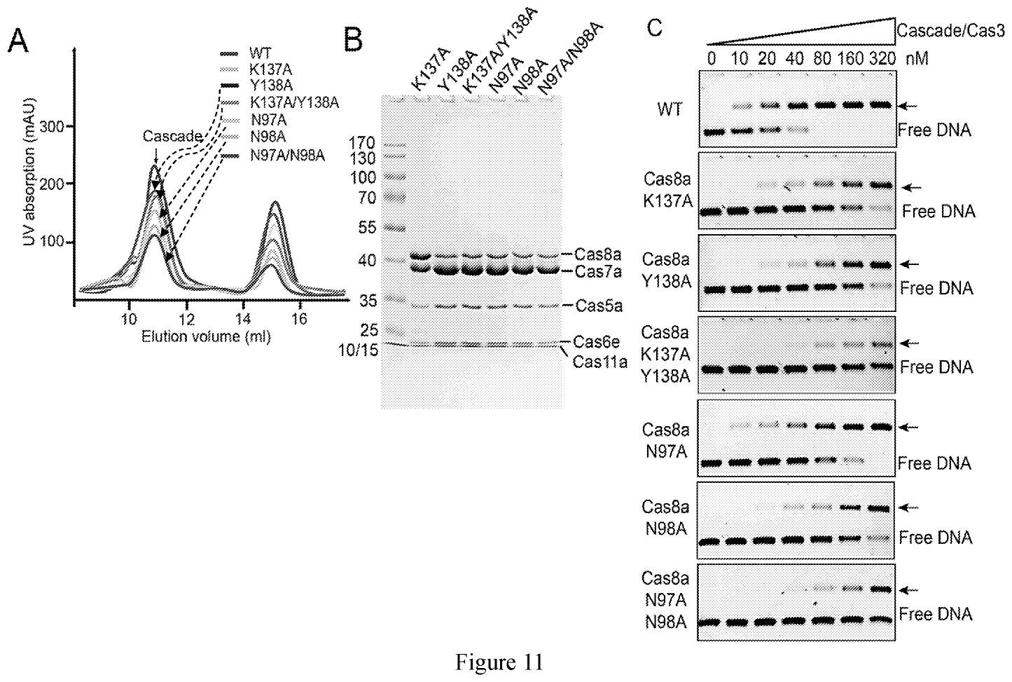

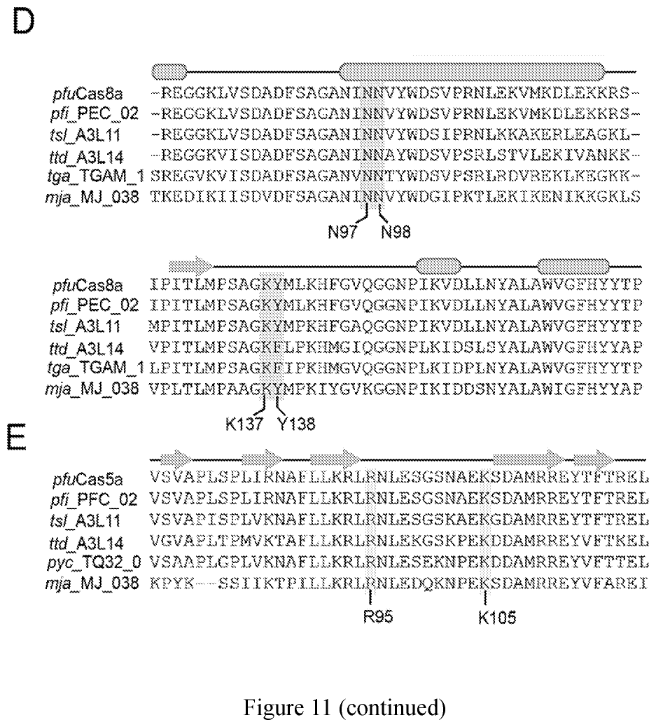

PAM recognition promotes RNA-guided DNA unwinding in DNA-targeting CRISPR systems. In Type I CRISPR systems, the process involves PAM recognition-induced DNA bending and the insertion of a Gln-wedge into DNA duplex to initiate unwinding (Xiao et al., 2017). In this disclosure we show that the same mechanistic principles hold true inside Type I-A Cascade-Cas3, despite the lack of structural similarity between Cas8a NTD and its counterpart in I-E and I-F Cascades. Pfu Cascade-Cas3 specifies a 5′-Y₋₃C₋₂N₋₁ PAM, which denotes a pyrimidine at PAM-3, a cytosine at PAM-2, and any nucleotide at PAM-1; a few alternative PAMs were also found to promote interference (Elmore et al., 2015). In both the intermediate and full R-loop structures, PAM is recognized by Cas8a NTD mainly from the DNA minor groove side and towards the target-strand DNA (3′-G₋₁G₋₂G₋₃). Consistent with the PAM code, no sequence specific interactions are found at PAM-1. The G₋₂-C₋₂ pair at PAM-2 is strongly specified from the minor groove side by a bidentate hydrogen bond from N97 to the sugar edge of G₋₂ and a weak H-bond from N98 to C₋₂. Alternative base-pairs do not satisfy the observed H-bonding pattern, which is consistent with the strict PAM-2 code. G-3 is specified from the major groove side by a polar contact to N7 from K137 (FIG. 4A). Both adenosine and guanosine suffice for this contact, which again is consistent with the PAM-3 code. Electrostatic contacts to the DNA sugar phosphate backbone are also found from Cas5a (K105, R95) and Cas8a (Y138, N149, and N98) (FIG. 4B). Mutations disrupting the observed sequence-specific contacts (K137A, Y139A, K137A/Y139A, N97A, N98A, N97A/N98A) reduced the target binding affinity by at least 10-fold (FIGS. 4C-D; 11A-C). Importantly, the PAM recognition residues are highly conserved among many Cas8a and Cas5a homologs (FIG. 11D-E), suggesting that a significant portion of the Type I-A CRISPR systems specify the same PAM code.

### Example 5

### Nuclease Activity of Cas3 is Highly Regulated

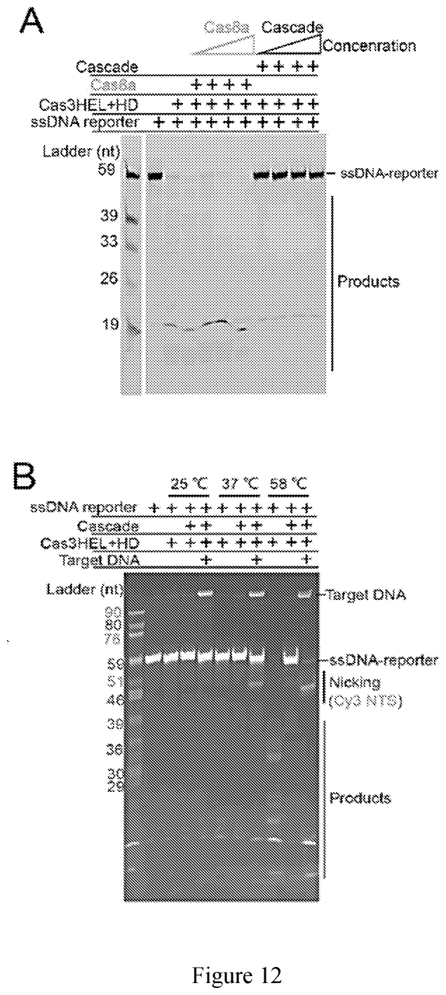

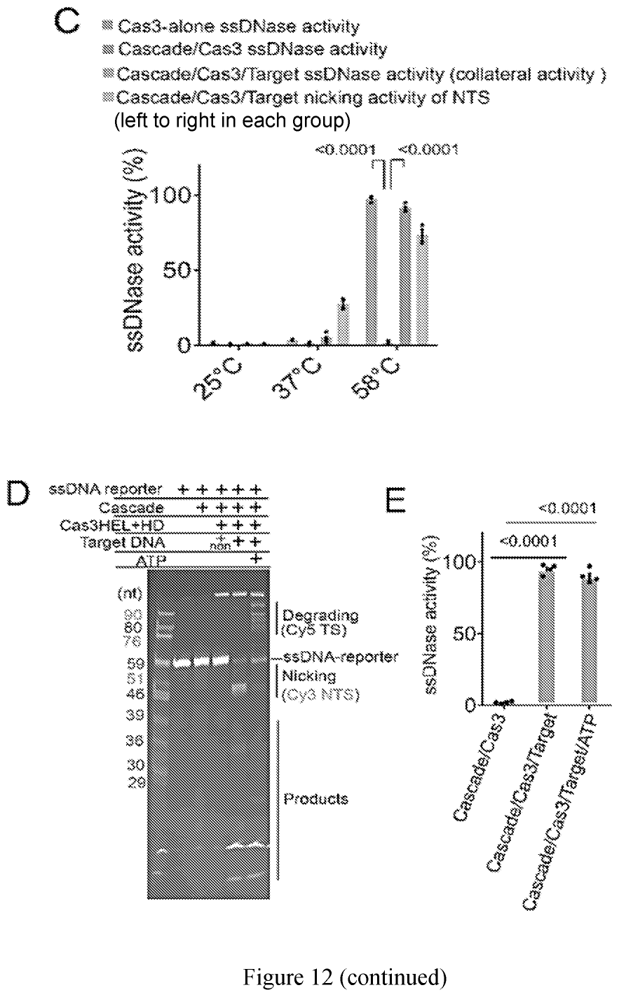

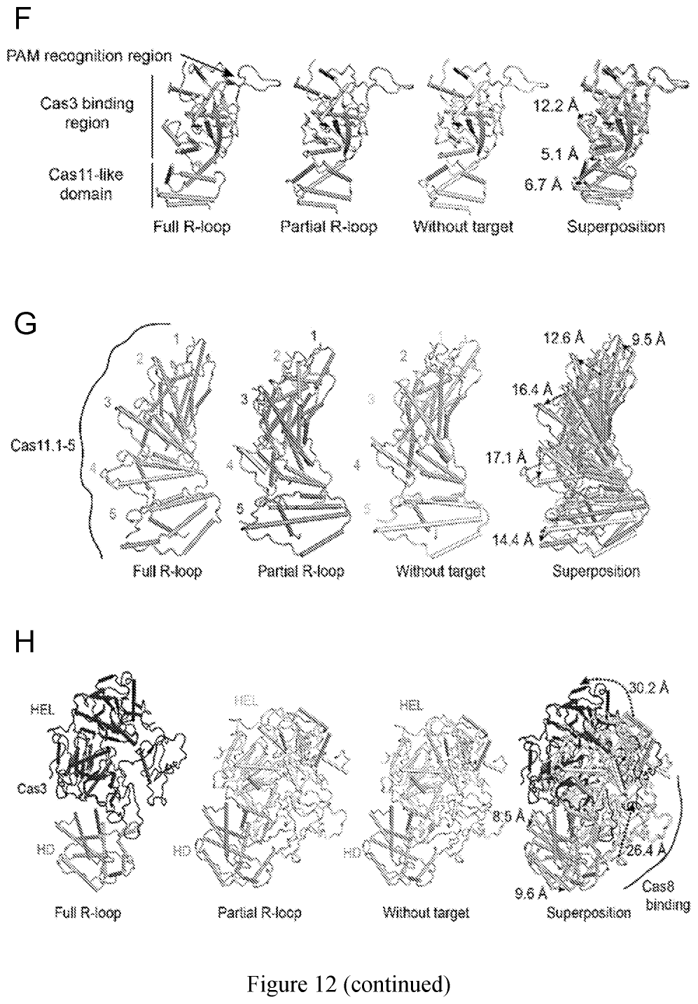

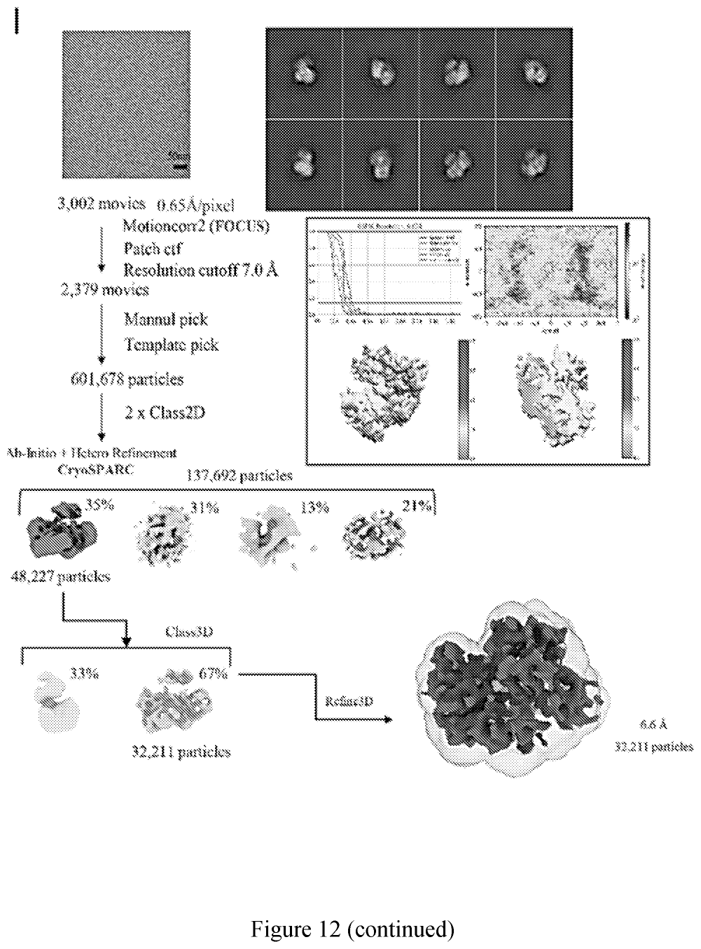

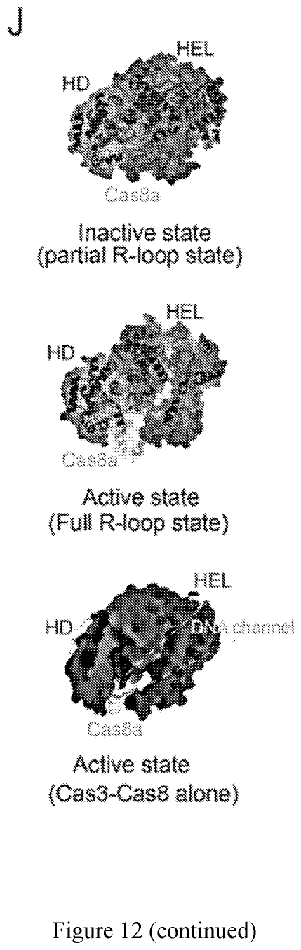

Work in I-E, I-C, and I-F systems suggest that Cas3 is recruited by Cascade in trans and only after R-loop formation. This conditional recruitment mechanism is considered to be important in preventing off-targeting (Szczelkun et al., 2014; Xiao et al., 2018; Xiao et al., 2017). Given that Cas3 binds constitutively to Cascade in the I-A system, an alternative mechanism must be in place to prevent off-targeting. The present disclosure shows that the nuclease activity of Cas3 is tightly regulated, in a Cascade- and DNA target-dependent manner. Pfu Cas3 was highly active in degrading the fluorescently labeled ssDNA reporter, either by itself or in complex with Cas8a (FIG. 12A). This nuclease activity against the ssDNA reporter became undetectable when Cas3 was present within the Pfu Cascade-Cas3 complex, only to be reactivated by the presence of a cognate dsDNA target (FIG. 12A-C). In such cases, the target DNA was efficiently nicked at the non-target strand, and further degraded in the presence of ATP (FIG. 12D-E). In contrast, noncognate DNA failed to activate Pfu Cascade-Cas3 against the ssDNA reporter (FIG. 12D). Collectively, the data suggest that the Pfu Cascade silences the nuclease activity of Cas3 upon complex formation, and only reinvigorates it upon encountering the cognate DNA substrate.

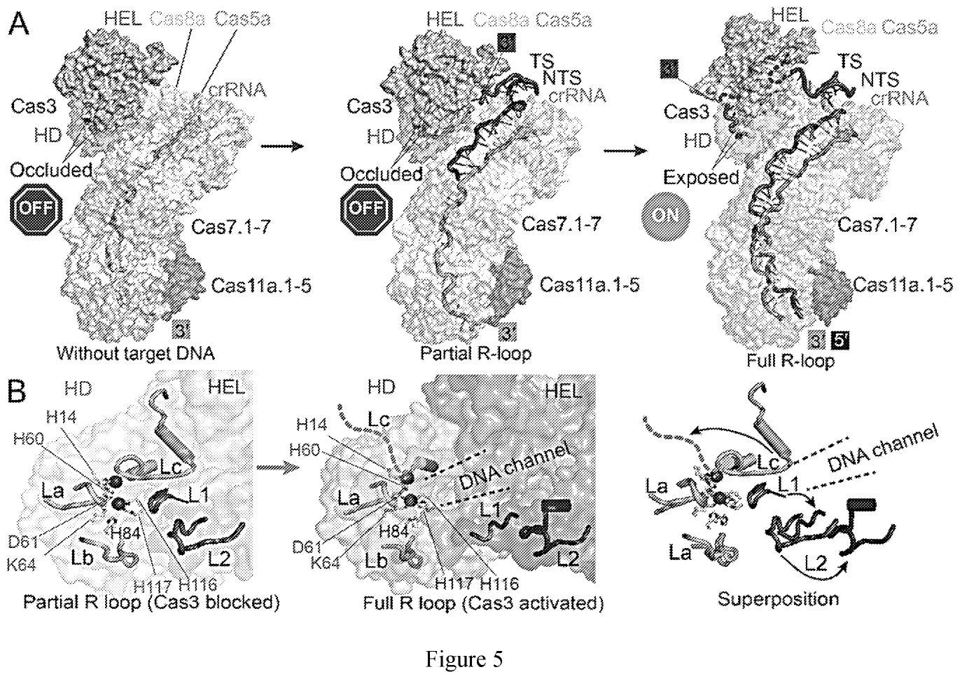

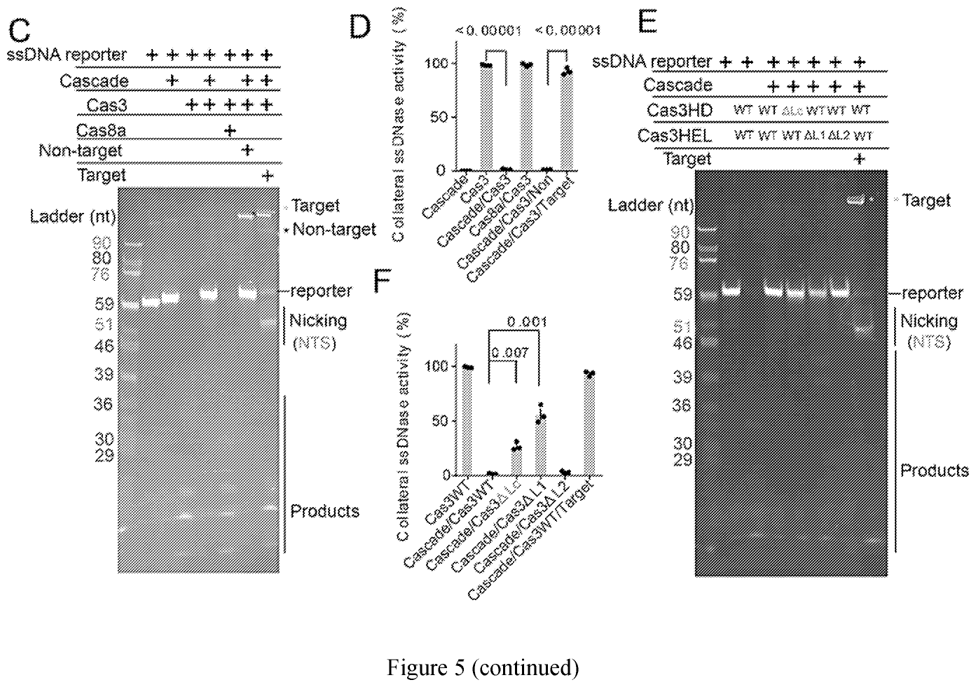

Capturing snapshots of Pfu Cascade-Cas3 before, in the middle, and after R-loop formation allowed us to deduce the nuclease regulatory mechanism in high resolution. The two early snapshots revealed that, prior to full R-loop formation, the HD nuclease center of Cas3 is occluded by the nearby structural features (FIG. 5A). Specifically, two loops (L1: R143-R148; and L2: L456-K474) from Cas3 HEL and one loop from the C-terminal region of Cas3 HD (Lc: L211-K237) juxtapose to form a wall structure. These loops not only block the direct entry of ssDNA into the HD active site, but also prevent the threading of ssDNA through the helicase into the HD nuclease (FIG. 5B). Therefore, even though a pair of Ni²⁺ ions are optimally coordinated for ssDNA cleavage (Huo et al., 2014), NTS nicking does not take place due to steric hindrance. This autoinhibition is relieved in the full R-loop state-L1 restructures from a β-hairpin to a short α-helix; L2 retracts due to a rigid body movement in its residing structure; and Lc becomes disordered. Both the direct-access route and the through-helicase route become unobstructed as the result (FIG. 5B).

The three conformational switches were individually deleted to evaluate their functional importance. The Cas3 nuclease activity was read out from the collateral cleavage of the fluorescent ssDNA reporter. The wild type Pfu Cascade-Cas3 showed undetectable nuclease activity in the absence of the cognate DNA target (or in the presence of a non-target dsDNA), but strong nuclease activity in the presence of a cognate DNA target (FIG. 5C-D). Similar to wild-type Cas3, L2 deletion (ΔL2) showed little collateral ssDNA cleavage activity in the absence of the cognate substrate, suggesting the autoinhibition mechanism is still intact. In contrast, ΔL1 and ΔLc mutants appeared to have lost the autoinhibition control. They displayed strong nuclease activity even in the absence of the DNA target (FIG. 5E-F). This constitutive-ON behavior would be quite detrimental because a partially matching DNA target could also be cleaved, leading to an unacceptable off-targeting scenario.

### Example 6

### Allosteric Regulation of Cas3 by Pfu Cascade, in High-Resolution

Structural comparisons further revealed that the conformational state of the Cas3 nuclease center is allosterically regulated by the Pfu Cascade in an R-loop dependent manner. Formation of a full R-loop, but not a partial R-loop intermediate, triggers a relay of conformational change in the inner belly of Pfu Cascade (FIG. 12F-H). The five Cas11a subunits and the Cas11-like Cas8a CTD twist in a concerted fashion, creating interdomain movements between Cas8a NTD and CTD (FIG. 125F-G). Structural changes are further relayed to Cas3 in the form of a global rigid-body motion and a local conformational change (FIG. 12H). The former orients the Cas3 helicase towards the substrate, and the latter unblocks the HD nuclease center (FIG. 5B). As the result, the non-target strand DNA is efficiently nicked, re-threaded through the helicase subunit, and further into the HD nuclease subunit (FIG. 5A, right panel).

To establish a stringent correlation between Cas3 activity and the HD active site conformation, we further resolved a 6.6 Å cryo-EM structure of the Pfu Cas3-Cas8a complex. Despite the moderate resolution, the EM densities clearly suggest the HD nuclease center is open and accessible, similar to that in the full R-loop state of Pfu Cascade-Cas3 (FIG. 1251-J). This structural observation is consistent with Pfu Cas3-Cas8a possessing high nuclease activity. It further supports the interpretation that the elaborate allosteric switch is set only upon complex formation with the intact Pfu Cascade.

### Example 7

### Mechanism-Inspired Development of a Nucleic Acid Detection Tool.

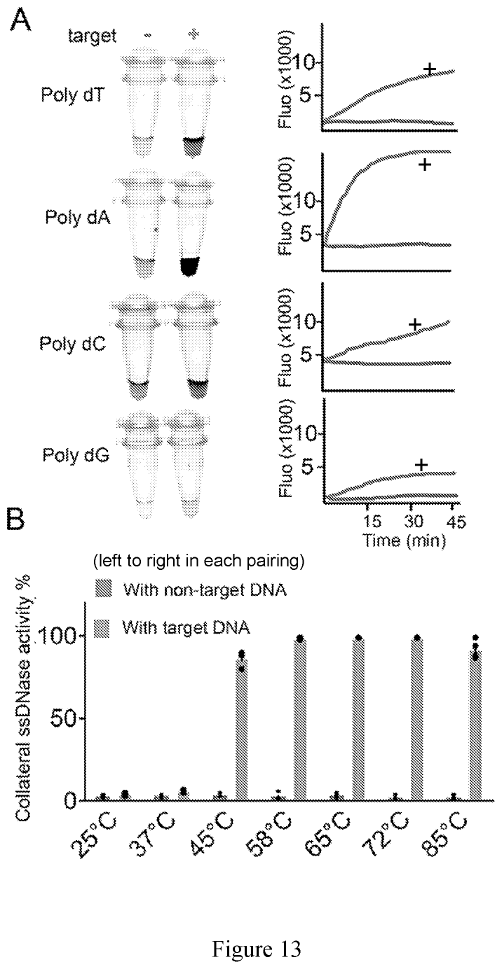

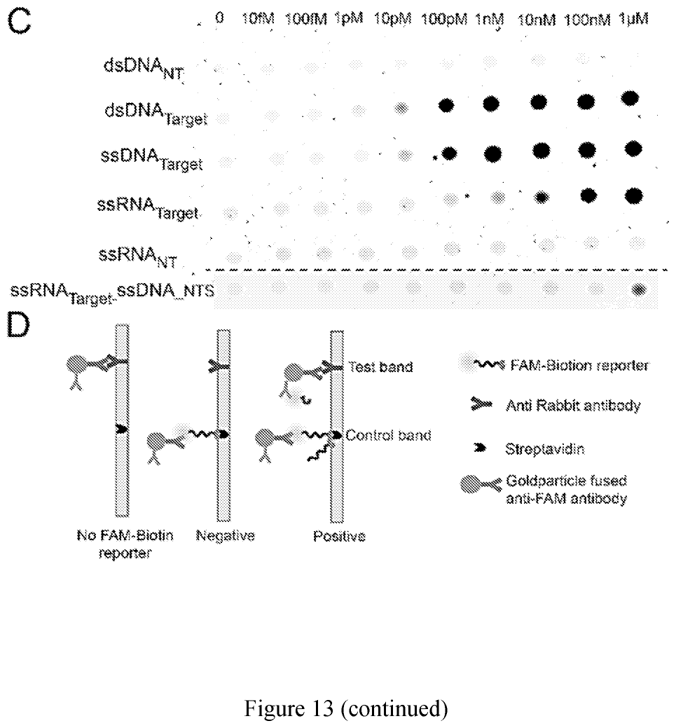

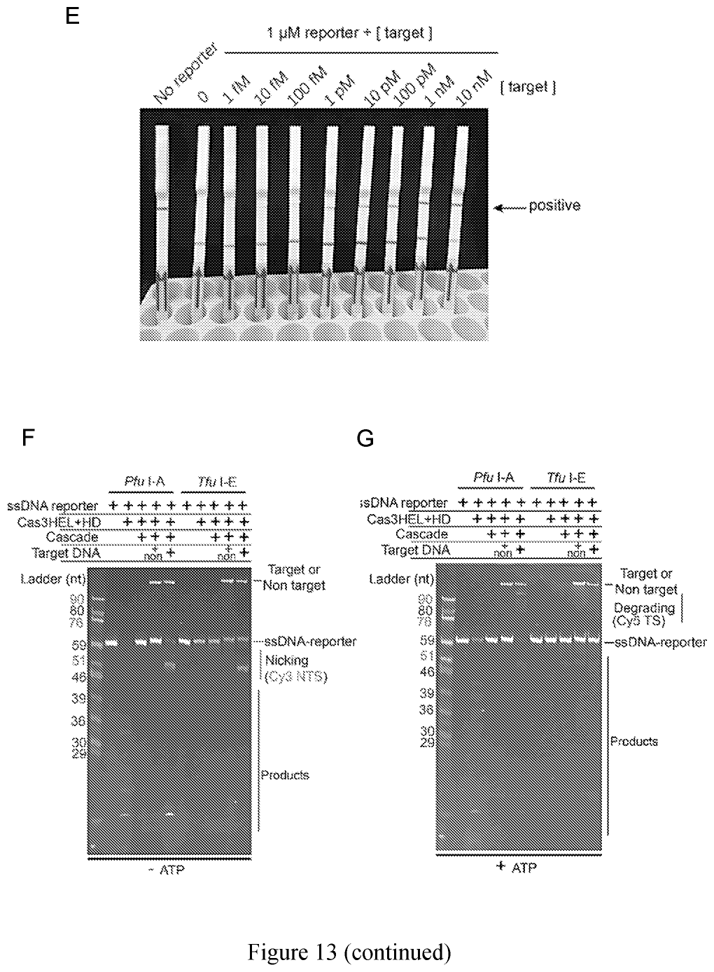

The foregoing data demonstrate that when a cognate DNA target is present, the nuclease activity of Pfu Cascade-Cas3 is not restricted to the DNA target; a ssDNA bystander is efficiently degraded as well (FIGS. 5C-D, 12B-D). The observed cleavage activity is analogous to the collateral damage activity reported for Cas12a (Chen et al., 2018; Gootenberg et al., 2018). The disclosure includes a nucleic acid detection format that does not rely on Cas12a. In this assay forma, a ssDNA containing a 5′-fluorophore and a 3′-quencher was used as a reporter (FIG. 6A). Cleavage of this reporter would relieve the quenching effect and produce fluorescent signals, which was indeed the case when Pfu Cascade-Cas3 was incubated with the target DNA, but not a non-target DNA (FIG. 6B). Pfu Cas3 alone produced high fluorescence regardless of dsDNA presence due to the lack of autoinhibition. Further analysis revealed that a poly-dA reporter produced bigger fluorescence changes than poly-dT, poly-dC, and poly-dG reporters (FIGS. 6C, 13A). Virtually no background fluorescence increase was observed when a non-target DNA was used over the course of one-hour incubation, which means this platform would be less prone to reporting false positives (FIG. 6D). We determined that the DNA cleavage reaction was heat-activatable. The signal amplification was rather small at 25 and 37° C., but became very robust throughout the 45-85° C. temperature range (FIG. 13B). This is also a desirable feature because it can facilitate cell lysis, pathogen inactivation, and nucleic acid detection to be combined into a single high-temperature incubation step.

Next, we investigated the substrate scope and detection limit of the Type I-A nucleic acid detection platform. In substrate titration experiments and with the fluorescence unquenching format, Pfu Cascade-Cas3 could reliably detect the presence of 1 pM dsDNA substrate in solution without the need of PCR amplification. Thus, in embodiments, the disclosure includes detecting as little as 1 pM dsDNA without amplification. Pfu Cascade-Cas3 displayed comparable detection sensitivity towards a ssDNA target, and ˜100-fold reduced sensitivity towards a ssRNA target (0.1-1 nM) (FIGS. 6E, 13C). These observations suggest that base-pairing to the entire spacer region of crRNA is sufficient to trigger the activation of Pfu Cas3; PAM recognition is not a prerequisite. In contrast, an RNA_TS/DNA_NTS heteroduplex target was only detected at UM range, which may reflect the combined consequence of poor PAM recognition and heteroduplex unwinding. This assay Type I-A nucleic acid detection platform is referred to as HASTE (heat-activated streamlined nucleic acid detection platform) because of its advantage in streamlining sample inactivation and nucleic acid detection into a 15-minute high-temperature incubation step (FIG. 6F). IT is considered that combining HASTE with isothermal amplification methods such as Loop-mediated isothermal amplification (LAMP) (Notomi et al., 2000) may further improve its detection limit. Alternatively, a 25-cycle PCR amplification step can be introduced prior to HASTE. As low as 1 aM dsDNA (single molecule level) can be robustly detected. No false positive or negative detection was observed among the twenty-two tested samples, over a wide substrate concentration range (FIG. 6G).

In parallel to the fluorescent-based assay, we also developed a lateral flow strip assay, where cleavage of the ssDNA reporter will allow its FAM-labeled 5′-portion to move beyond the streptavidin-coated control line, and to carry the anti-FAM antibody coated gold nanoparticle to the secondary antibodies coated test line (FIG. 13D). By systematically titrating the target DNA, we determined that the sensitivity of this detection method is in the ˜10-100 fM range without amplification (FIG. 13E), which is comparable with Cas12 in similar setups (Gootenberg et al., 2018). Collectively, the evidence indicates that type I-A Pfu Cascade-Cas3 has the potential to be engineered into a robust and sensitive nucleic acid detection tool.

We further investigated whether a similar nucleic acid detection platform can be developed from Thermobifida fusca Type I-E Cascade-Cas3, which has been extensively characterized by us (Dillard et al., 2018; Dolan et al., 2019; Huo et al., 2014; Xiao et al., 2018; Xiao et al., 2017). Results showed that Tfu Cascade-Cas3 in the same testing format suffered many problems. In the absence of ATP, Tfu Cascade-Cas3 showed high background noise in the absence of targets (unstable baseline) and low sensitivity (small dynamic range) in the presence of targets. In the presence of ATP, the collateral damage activity from Tfu Cascade-Cas3 was greatly reduced. However, very little fluorescent signal was observed in the presence of the cognate DNA target (FIGS. 6H-I, 13F-G). The background problem would lead to rampant false positives, and the sensitivity issue reduces the detection limit. These results show that developing a nucleic detection platform from other Type I systems would be difficult and require extensive experimentation and optimization.

### Example 8

### Converting Pfu Cascade-Cas3 to a Highly Efficient and Heat-Inducible, Gene Deletion-Editing Tool.

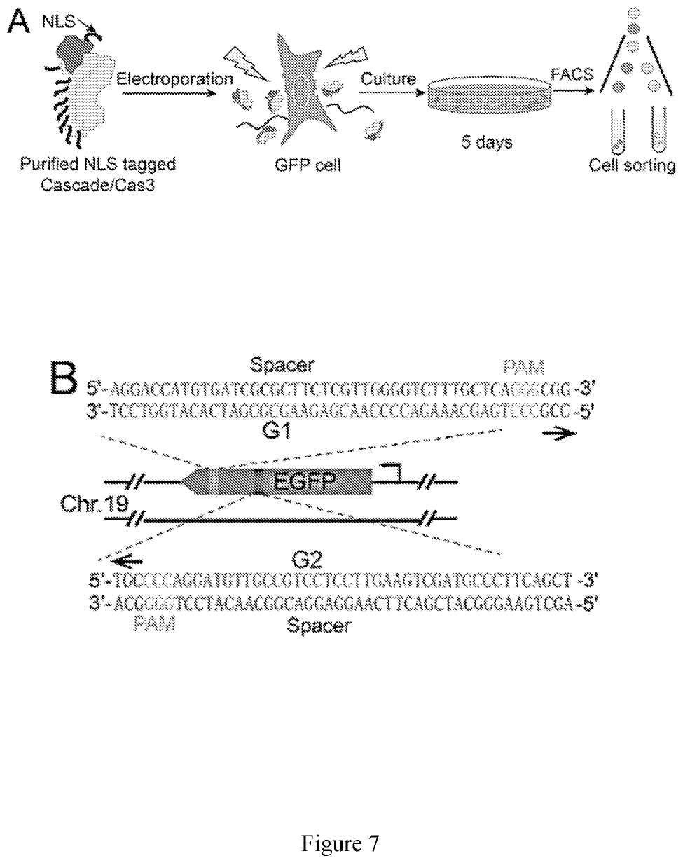

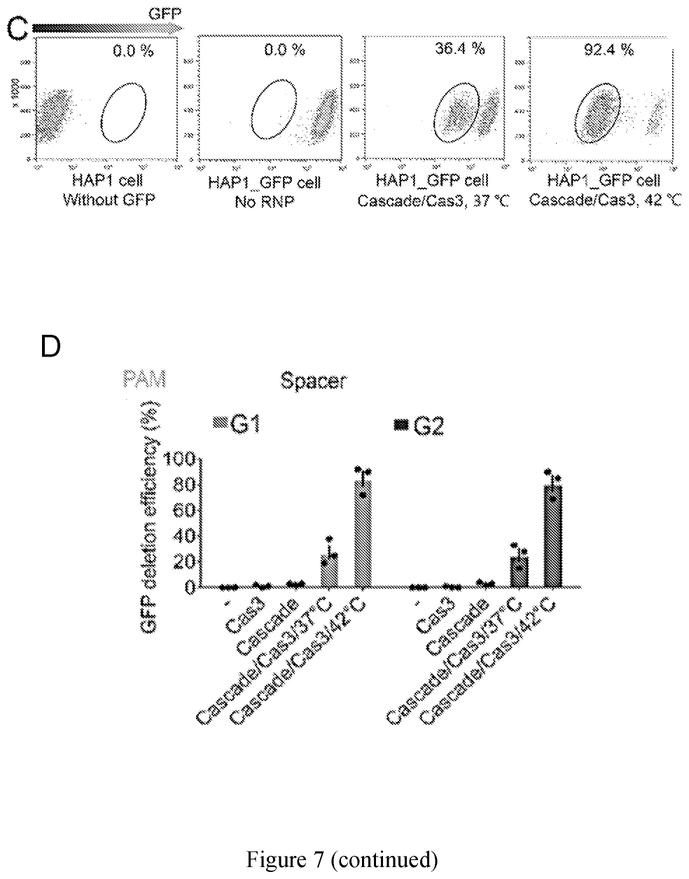

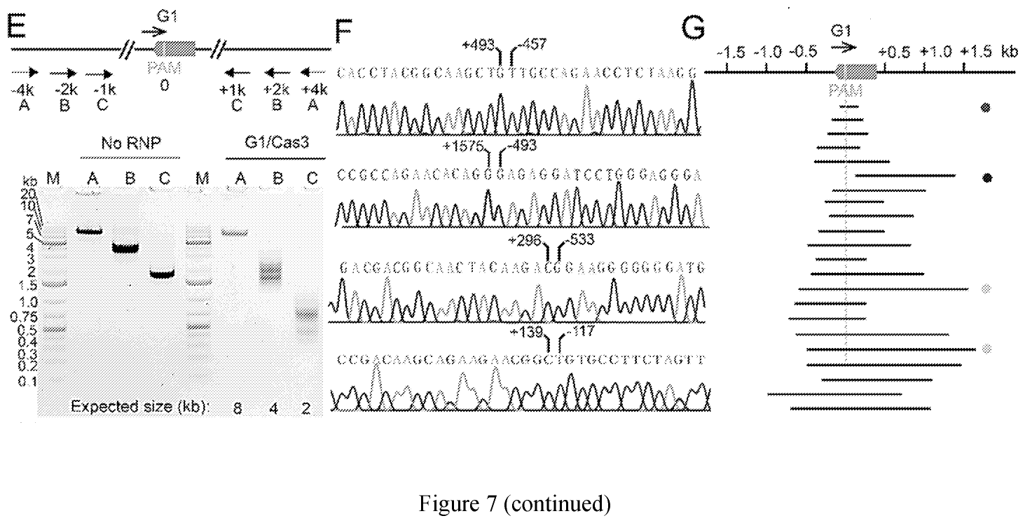

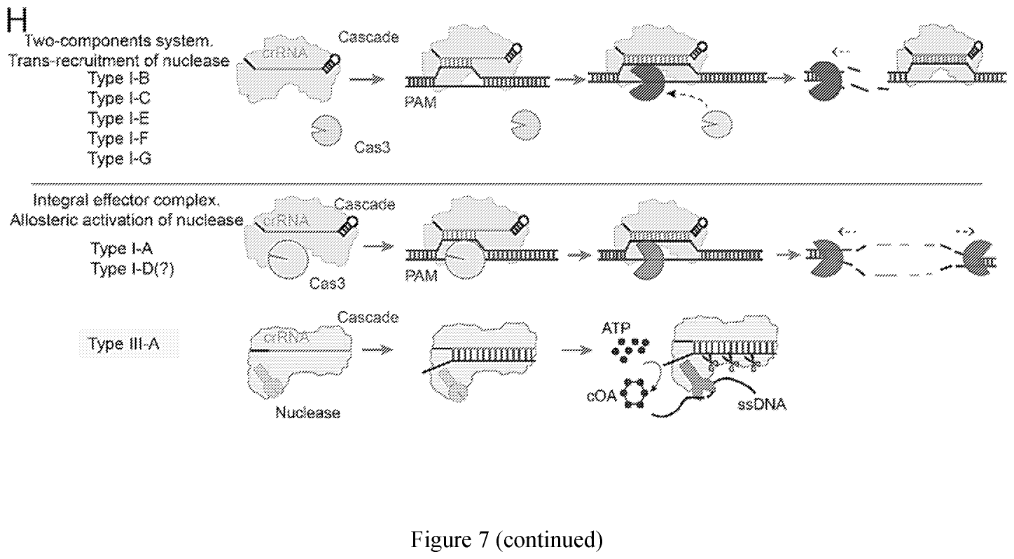

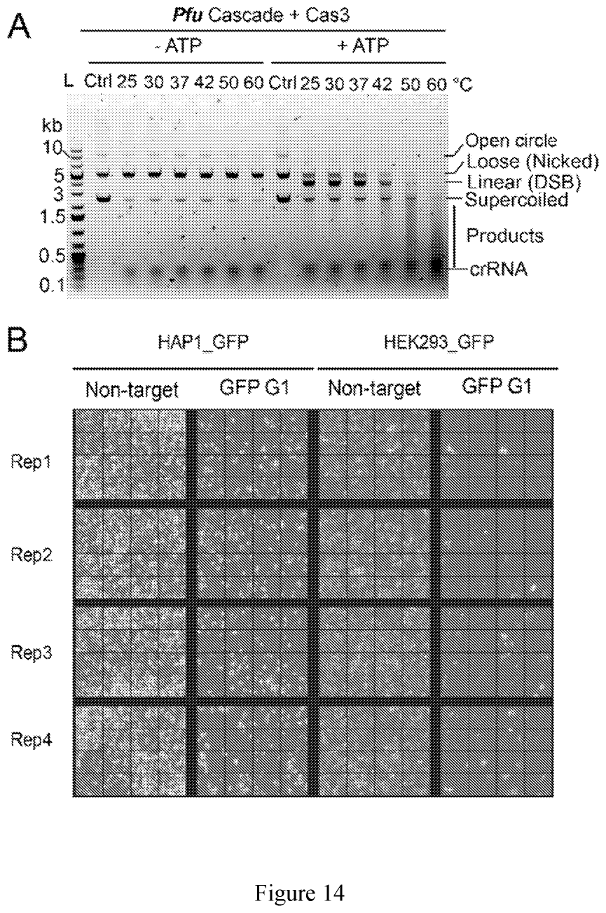

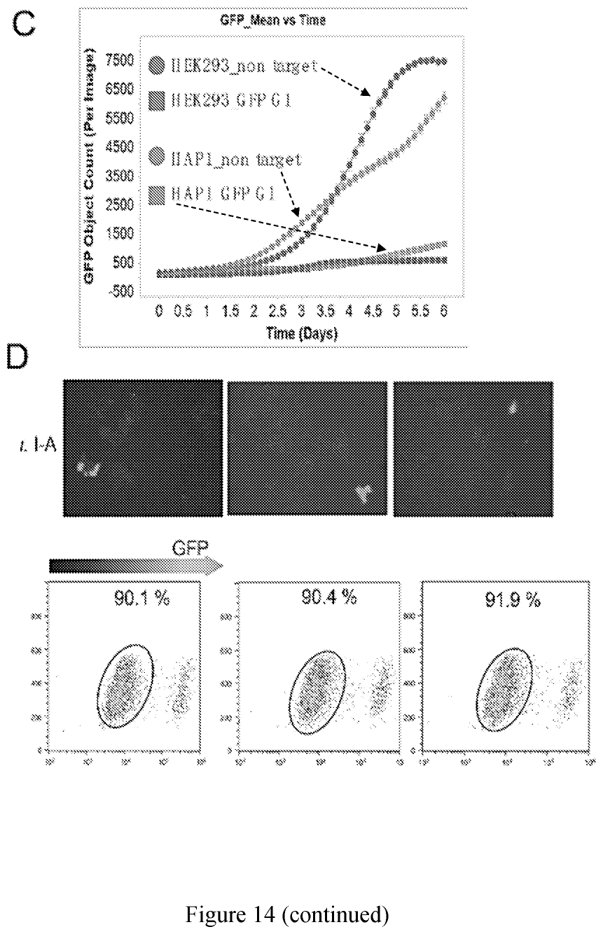

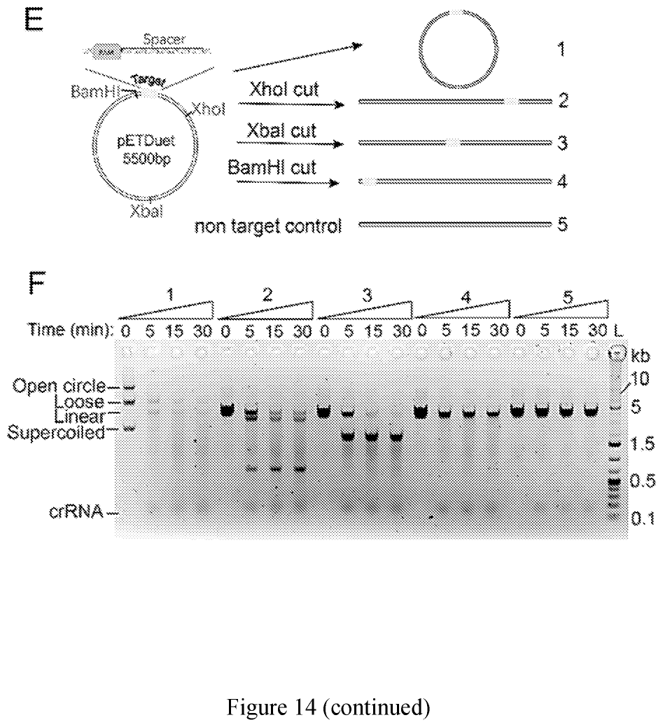

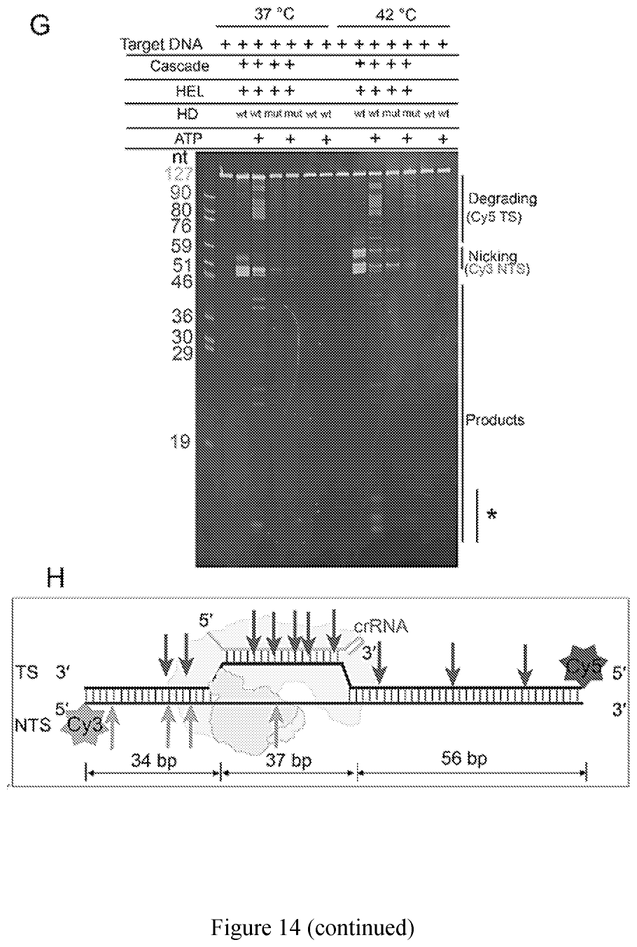

We developed the described I-A CRISPR-Cas system for genome editing in human cells. Nuclear localization signal (NLS) tagged Pfu Cascade-Cas3 was delivered as ribonucleoprotein complexes (RNPs) into cells through electroporation. This approach allowed us to establish a direct correlation between in vitro RNA-guided nuclease activity and in vivo editing efficacy. The experiments were carried out in a HAP1 cell line that has spontaneously reverted from haploid to the diploid forms, and with a single copy of eGFP gene inserted into the AAVS1 safe harbor in Chromosome 19. Knowing that the RNA-guided nuclease activity of Pfu Cascade-Cas3 increases significantly from 37 to 42° C. (FIG. 14A, 10A-B), we analyzed ex vivo editing at both temperatures (FIG. 19C-F), and quantified the editing efficiency using Fluorescence-Activated Cell Sorting (FACS) (FIG. 7A). At 37° C., ex vivo editing against the template strand of eGFP by guide 1 (G1) led to the loss of GFP signal in 36.4% of the cell population. The same experiment at 42° C. led to almost quantitative GFP signal loss (92.4% editing efficiency quantified by FACS) (FIG. 7B-C). To evaluate whether RNA transcription influences editing efficiency, the same experiment was repeated using Pfu Cascade-Cas3 programmed against the non-template strand of eGFP by guide 2 (G2). A similar temperature-dependent editing behavior was observed, and the editing efficiency at 42° C. was comparable (90%). Combining Guide 1 and Guide 2 RNPs slightly improved the editing efficiency (FIG. 19F). Shortening the 42° C. incubation from 24 to 8 hours after Pfu Cascade-Cas3 delivery gave comparable editing efficiency (data not shown). Thus, the disclosure includes the described incubation times for use with cells that cannot can withstand extended incubation at 42° C. We further expanded the GFP-editing experiments to a GFP-tagged HEK293 cell line and observed similar editing efficiency (FIGS. 14B-C). This confirms that I-A Pfu Cascade-Cas3 is a highly efficient genome editing tool in human cells. Notably, delivering excess amount of Pfu Cas3 did not further improve the editing efficiency of Pfu Cascade-Cas3, which is consistent with the notion that Cascade and Cas3 function as an integral effector complex in Type I-A CRISPR systems. (FIG. 14D). The high editing efficiency and temperature-tuned editing activity are two representative advantages for the described system.

Next, we examined the editing profile of Pfu Cascade-Cas3 using PCR amplification, cloning and Sanger sequencing (FIG. 7E-F). Several Type I CRISPR systems have been developed for deletion-editing in human cells (Cameron et al., 2019; Dolan et al., 2019; Morisaka et al., 2019; Osakabe et al., 2020; Osakabe et al., 2021). The majority of them are unidirectional editors, causing long-range deletions upstream of the PAM-side of the target DNA. Results of this disclosure showed that the Type I-A Pfu Cascade-Cas3 behaves as a bi-directional deletion editor. Of the 22 clone sequences, all but one deleted a continuous region encompassing both the upstream and downstream of the target site (FIG. 7E-H). Upon observing this deletion profile, we further investigated the deletion profile of Pfu Cascade-Cas3 using in vitro assays. In plasmid cleavage assays, restriction mapping revealed that both the upstream and downstream regions of the targeted plasmid were erased (FIG. 14E-F). In dual-labeled linear DNA degradation assays, cleavage sites was mapped to both sides of the R-loop (FIG. 14G-H). Therefore, the observed deletion-editing profile is a direct reflection of the biochemical activity of Pfu Cascade-Cas3.

It will be recognized from the foregoing that one aspect of this disclosure is the unveiling of an alternative interference mechanism employed by the I-A CRISPR system, where Cas3 is constitutively associated with Cascade, and its activity is allosterically regulated by Cascade, in an RNA-guided and DNA substrate-dependent fashion. By capturing four functional states of the I-A Cascade-Cas3 effector complex using cryo-EM, we established strong causal relationships between structural changes and functional consequences. Binding to Cascade sets on a conformational switch in Cas3 to lock its HD nuclease center. This autoinhibition is relieved only upon full R-loop formation through a substrate-binding induced conformational relay from Cascade to Cas3. The disclosure includes categorizing Type I systems into the allosteric-activation group and the trans-recruitment group (FIG. 7H). This allosteric-activation mechanism likely also exists in the I-D system, where Cas3 HD is fused to the Cas8d subunit of Cascade (Makarova et al., 2020). However, the available structure-function analysis suggests the I-D cascade binds to target DNA efficiently in the absence of Cas3 HEL (McBride et al., 2020). Therefore additional studies are needed to accurately classify the mechanism in the I-D subtype. I-B and I-G have not been extensively studied.

We further developed the HASTE nucleic acid detection method based on the I-A interference mechanism. This platform has advantages over previously available detecting methods, including being heat-activatable, streamlined, sensitive yet accurate, and broad-spectrum (i.e, the described assay can detect dsDNA, ssDNA, and ssRNA). HASTE is expected to be suitable for use for bacterial and viral pathogen detection where diagnostic time and biosafety is on high demand. This detection platform includes strong autoinhibition of Cas3 HD within Cascade-Cas3 complexes to maintain a low false-positive background. The described system produces bi-directional genome editing profile. This is an unexpected discovery as the helicase activity of Cas3 is unidirectional (Dillard et al., 2018; Redding et al., 2015).

### Example 9

This Example provides materials and methods used to produce the results described herein.

STAR*METHODS
KEY RESOURCES TABLE

| | | |
|---|---|---|
| Reagent or resource | Source | Identifier |

Bacterial Strains

| | | |
|---|---|---|
| E. coli Bl21(DE3) competent cells | NEB | Cat# C2527I |
| E. coli DH5a competent cells | NEB | Cat# 18265017 |

Chemicals, Peptides, and Recombinant Proteins

| | | |
|---|---|---|
| Modified and unmodified oligos | IDT | N/A |
| IPTG | GoldBio | Cat # 12481C500 |
| LB broth | Teknova | #L9145 |
| Restriction enzymes | NEB | |
| iProof high fidelity PCR kit | Biorad | |
| Acrylamide | thermal | |
| Urea | VWR | |
| Fluorescence stain Cy3/Cy5 | Lumiprobe | |
| SYPRO ® Orange | Thermo Fisher | |
| Formamide | VWR | |
| MnCl₂ | Fisher Scientific | |
| Strep resin | IBA | |
| Nickel resin | QIAGEN | |
| lateral flow strip | HybriDetect 1 | MGDS |
| Superdex 200 increase | GE | |
| Copper Grids 1.2/1.3 200 mesh | QUANTIFOIL | Q250-CR1.3 |

### Experimental Model and Subject Details

Escherichia coli BL21 (DE3)
E. coli BL21 (DE3) cells were used for protein production. Cells were grown in Lysogeny Broth (LB) supplemented with appropriate antibiotics.
Escherichia coli BL21 AI
E. coli BL21 AI cells were used for assaying for CRISPR interference by the Pfu I-A system.
Cells were grown in Lysogeny Broth (LB) supplemented with appropriate antibiotics. Escherichia coli DH5 alpha
E. coli DHSα was used for cloning. Cells were grown at 37 C in LB supplemented with appropriate antibiotics.
HAP1 cell culture
This GFP-tagged diploid HAP1 cell line was a gift from Yan Zhang's lab (Tan et al., 2022). Cells were cultured in DMDM (Gibco) supplemented with 10% FBS (Gibco) at 37° C. and 5% CO2 in a humidified incubator. Cells were suspended using Trypsin-EDTA solution (GIBCO) and split every 2 to 3 days.
HEK293 cell culture
HEK293 cells were cultured in DMDM (Gibco) supplemented with 10% FBS (Gibco) at 37° C. and 5% CO2 in a humidified incubator. Cells were suspended using Trypsin-EDTA solution (GIBCO) and split every 2 to 3 days.

### Method Details

### Main Plasmids Construction

Plasmids, primers, and RNA guides used in this work are listed in Supplementary Tables 1, 2, and 3, respectively. Cloning was performed in E. coli DH5a. The Type I-A cas operon from Pyrococcus furiosus DSM 3638 was PCR-amplified using the iproof Polymerase (BioRad) and cloned into the pCDFDuet vector, giving rise to Plasmid pCascade/Cas3. For plasmid pCRISPR (or pcrRNA), a series of synthetic constructs composed of T7 promotor, CRISPR array (repeat-spacer-repeat with Cas6 or ribozyme derived 5′ handle-spacer) were introduced into the high copy number vector pRSFDuet. For purification, cas8a was cloned into pCDFduet with a N-terminal Twin-strep tag. cas11-cas7-cas5 operon was inserted into pETDuet with a C-terminal His tag on Cas5a. Cas3 HD and Cas3 HEL were expressed individually from the pRSFDuet vector with a N-terminal His tag. All plasmids were verified by Sanger sequencing. Bacterial transformations were carried out using chemically competent cells, and transformants were selected on LB agar plate supplemented with the appropriate antibiotics.

Cloning, expression and purification pCDFDuet-Twin-Strep-Cas8a (Strep^{R}), pETDuet-Cas11-Cas7-Cas5 (Amp^{R}) and pcrRNA-Cas6 (Kan^{R}) were co-transformed into E. coli BL21 (DE3) cell under the appropriate antibiotic selection. A 4 liter cell culture was grown in LB medium at 37° C. until an optical density of 0.6 at 600 nm. The culture temperature was then reduced to 20° C. and incubated for an additional 1 hour. Expression was induced with 0.5 mM isopropyl-β-D-thiogalactopyranoside (IPTG) at 20° C. overnight. Cells were harvested by centrifugation and resuspended in 100 mL buffer A containing 50 mM HEPES pH 7.5, and 300 mM NaCl, 10% glycerol, and 2 mM β-ME. Cells were lysed by sonication, and the debris was cleared using centrifugation at 12,000 rpm for 50 min at 4° C. The supernatant was applied onto a pre-equilibrated 4 mL streptavidin column (Twin-strep purification). After washing with 50 ml of buffer A, the protein was eluted with 20 ml buffer B (50 mM HEPES pH 7.5, 300 mM NaCl, 10% glycerol, 2.5 mM desthiobiotin). The sample was then concentrated to 1 ml and loaded onto a Superdex 200 16/60 size-exclusion column (GE Healthcare) equilibrated with buffer C (10 mM HEPES pH 7.5, 300 mM NaCl), the peak fractions of Cascade complex were pooled and snap-frozen in liquid nitrogen for later usage.

For Cas3 HD and Cas3 HEL purification, pRSFDuet-Cas3HD or Cas3HEL (Kan^{R}) was individually transformed into E. coli BL21 (DE3), expressed using the same procedure as described above. Cells were harvested by centrifugation and lysed by sonication in 80 ml of buffer A containing 50 mM HEPES pH 7.5, 500 mM NaCl and 20 mM imidazole, 10% glycerol, and 2 mM β-ME. The lysate was centrifuged at 12,000 rpm for 50 min at 4° C., and the supernatant was applied to a pre-equilibrated 4 mL Ni-NTA column. After washing with 100 ml of buffer A, the protein was eluted with 20 ml buffer B (50 mM HEPES pH 7.5, 500 mM NaCl, 10% glycerol, 300 mM imidazole, and 2 mM B-ME), concentrated to 1.5 mL and further purified on Superdex 200 16/60 equilibrated with buffer C (10 mM HEPES pH 7.5, 300 mM NaCl), the peak fractions were pooled and snap-frozen in liquid nitrogen for later usage.

### RNA Extraction and Ureal Gel Running

20 μL of Cascade sample at 2 μM and 20 μL phenol-chloroform solution was mixed together and vortexed vigorously for 2 min at room temperature. The aqueous and organic phases were separated by 13,000 rpm centrifuge for 15 min at room temperature. 10 μL sample was taken from the aqueous phase (top layer), mixed with 15 μL of formamide loading dye, heat-denatured at 95° C. for 10 min, and immediately loaded to a 12% ureal-polyacrylamide (PAGE) gel. After 50 minutes of electrophoresis at 25 watts, the gel was stained with EtBr to for 10 min, destained in water for 10 minutes, and scanned with the ChemiDoc imaging system (Bio-Rad) at appropriate wavelength.

### In Vivo Assay for Cascade and Cas3-Mediated Interference

pCDFduet-Cascade/Cas3 (StrepR), pRSFDuet-crRNA-Cas6 (KanaR) and pETDuet-Targets (AmpR) with different PAM sequence were co-transformed into the E. coli BL21AI cell line and grown on LB agarose plates containing kanamycin (50 μg/ml), ampicillin (100 μg/ml), streptomycin (30 μg/ml). After transformation, a single colony were cultured at 37° C. in nonselective LB medium (Strep+Kana) to O.D.600 of 0.3, at which point the expression of PfuCascade, Cas3, and pre-crRNA was induced for 12 h by the addition of 0.5% L-arabinose and 1 mM IPTG. Each cell culture was then divided into two equal volumes and plated onto Strep+Kana+Amp LB plates (selective for pETDuet-target) and Strep+Kan plates (non-selective for the target plasmid) in a series of dilutions. The number of colonies on each plate was counted after overnight incubation at 37° C. The CRISPR interference efficiency was reflected in the ratio of colony-forming units on the nonselective over selective plates. Each experiment was repeated three times to calculate the s.d. The experimental procedure is illustrated in FIG. 8K and Table 1.

### Fluorescently Labeled Prespacer Substrate Preparation

Fluorescent DNA oligos (Supplementary Table 2) for biochemistry were synthesized (Integrated DNA Technologies) with either a 5AmMC6 or 3AmMO label, fluorescently labeled in-house by Cy3 or Cy5-NHS dye (Lumiprobe), and annealed at equimolar amount, and native PAGE purified to remove unannealed ssDNA.

### Electrophoretic Mobility Shift Assay

5 nM final concentration of fluorescently labeled target DNA was incubated with titrations of Pfu Cascade or Cas3/Cascade complex in a 20 μL total reaction volume containing 50 mM Tris pH 8.0, 150 mM NaCl, and 10% glycerol. After a 15-minute incubation at 42° C. or specified condition in the figure legends, 10 μL of each sample was loaded onto 1% agarose gel equilibrated in 0.5×TBE buffer. Electrophoresis was performed at 60 V for 40 min in cold room. The fluorescent signals from the gel were recorded using a ChemiDoc imaging system (Bio-Rad).

For the fluorescence labeled protein EMSA assay in FIG. 1C., the proteins were labeled using corresponding fluorophore (Sulfo-Cy3 or Cy5, Lumiprobe) following our previously published protocol (Xiao et al., 2017). The 20 μL binding experiments used 300 nM Cy3-Cas3, 100 nM Cy5-Cascade, and 20 nM FAM-DNA in single tube or combined two or three components in one tube. Incubation and electrophoresis were carried out as above.

Plasmid Transformation Assay in P. furiosus

P. furiosus strains were generated via the previously described pop-in/pop-out marker technique (Elmore et al. 2016). Cultures and media were prepared as described previously (Lipscomb et al. 2011). Incubations were performed under anaerobic conditions at 95° C. in defined P. furiosus media. Liquid cultures were grown to mid to late log phase, and 200 μL of culture was combined with 100 ng of plasmid DNA (in 4.0 μL). The mixtures were plated on solid defined media and incubated for ˜62 h. Following incubation, colonies on each plate were enumerated, and transformation efficiency (Colony Formation Units/μg plasmid DNA) was calculated and plotted logarithmically. This assay was carried out with a minimum of three replicates.

### Affinity Pull-Down Assay

15 μg of strep-tagged Pfu Cascade and 15 μg of untagged Pfu Cas3 complex were mixed and incubated with 10 μL of strep resin at 4° C. for 30 min in a binding buffer (50 mM HEPES pH7.5, 10% glycerol, 300 mM NaCl), in a total assay volume of 50 μL. The strep resin was pelleted by centrifugation at ˜100 g for 30 seconds, washed 3 times with 200 μL of the corresponding binding buffer, then eluted with 70 μL of elution buffer (50 mM HEPES pH7.5, 300 mM NaCl, 2.5 mM desthiobiotin and 10% glycerol). Eluted proteins were separated on 12% SDS-PAGE and stained by Coomassie blue.

### Cascade-Mediated Cas3 DNA Cleavage Assay and Collateral Activity Assay

The 127 bp dsDNA substrate was produced from PCR using 5′-fluorescently labeled primers (Cy3 at NTS and Cy5 at TS). The reaction mixture was prepared from 100 nM final concentration of Pfu Cascade, 100 nM Pfu Cas3 (HEL+HD) and 10 nM substrate in a cleavage buffer containing 10 mM HEPES pH 7.5, 150 mM NaCl, 10 mM MgCl₂ and 100 μM CoCl₂. The reaction was incubated at 58° C. (or indicated temperatures in the figures) for 30 min. The collateral cleavage activity of pfu Cas3 used 10 nM FAM-ssDNA reporter instead. After incubation, the nucleic acids were phenol-chloroform extracted and separated on a 12% denaturing polyacrylamide gel. Fluorescent signals were recorded using a Typhoon™ scanner (Amersham).

### HASTE Detection Assay

22 samples with the corresponding concentration of target plasmid were mixed with the PCR system by introducing primers and iProof polymerase in 50 μl total reaction volume. After 25 cycles (this step is taken for 15 min), 1 μl of each sample was combined with 20 μl HASTE tool (100 nM final concentration of Pfu Cascade, 100 nM Pfu Cas3 and 100 nM ssDNA-FQ reporter) respectively, and incubated at 60° C. for 15 min. Finally, all reaction tubes were scanned using a BioRad ChemiDoc imager. For the comparison, all samples were also resolved after electrophoresis on 1% agarose gels.

### Lateral Flow Assay for Pfu Cascade-Cas3 Based Nucleic Acid Detection

20 μL cleavage reactions were prepared and incubated at 58° C. for 30 min with 1 μM final concentration of FAM-ssDNA-biotin reporter. Afterwards, 10 μL 30% PEG 6k and 30 μL of HybriDetect 1 assay buffer (Milenia) was added to the reaction and allowed to diffuse on a HybriDetect 1 lateral flow strip (Milenia) for 5 min.

### RNP Electroporation of GFP-HAP1 and GFP-HEK293 Cells

The GFP-tagged diploid HAP1 cell line was a gift from Yan Zhang's lab (Tan et al., 2022). The GFP-tagged HEK293 cell line was purchased from GenTarget. The two cell lines were maintained in similar fashion, in DMDM (Gibco) supplemented with 10% FBS (Gibco) at 37° C. and 5% CO2 in a humidified incubator. The cells were electroporated using the Neon Transfection system (ThermoFisher) according to the manufacturer's instructions. Briefly, HAP1 cells were individualized with 0.05% Trypsin-EDTA solution (GIBCO), washed once with DMDM (*give description), 10% FBS and resuspended in Neon buffer R to a concentration of 5×10⁶ cells/mL. 20-40 pmol of NLS-Pfu Cascade/NLS-Pfu Cas3 complex were mixed with approximately 5×10⁴ cells in buffer R (Neon Transfection system) in a total volume of 14 μL. Each mixture was electroporated using a 10 μL Neon tip (1450 V, 13 ms, 4 pulses) and plated in 6-well tissue culture plates containing 2 mL IMDM, 10% FBS.

### Flow Cytometry Analysis, FACS Sorting and Single Cell Isolation

Cells were individualized with 0.05% Trypsin-EDTA solution (GIBCO) 5 days after electroporation and resuspended in 1×PBS before experiments. For analysis, individualized cells were analyzed on a BD Biosciences FACSAria Fusion using the 488 nm laser for EGFP. Data analysis was performed using FlowJo® v10.4.1 in Flow Cytometry Facility of Cornell university. Sorted cells were then cultured in tissue culture incubator with 5% CO₂ at 37° C. for other usage.

### DNA Lesion Analysis by Long-Range PCR Genotyping

Genomic DNA of HAP1 cells were isolated using Gentra Puregene Cell Kit (QIAGEN) per manufacturer protocol. Long-range PCRs were all done using iProof DNA Polymerase (Biorad). Products were resolved on 1% agarose gels, stained by EtBr dye and visualized with Chemidoc MP imager (Biorad). See Supplementary Table 2 for all primers used for long-range PCRs. To define lesion junctions shown in FIGS. 7, lesion PCR reactions were purified using QIAquick PCR Purification Kit (QIAGEN), cloned into pJET vector (Thermo fisher), and transformed into DH5a cells. Plasmids from randomly picked single colonies were Sanger-sequenced to define the deletion boundaries.

### Cryo-EM Data Acquisition

4 μL of 0.6 mg/mL SEC-purified complexes were applied to a Quantifoil holey carbon grid (1.2/1.3, 400 mesh), which had been glow-discharged for 30 sec at 30 mA current. Grids were blotted at 8° C. for 4 seconds with zero force setting, 100% humidity and plunge-frozen in liquid ethane using a Mark IV FEI/Thermo Fisher Vitrobot. Cryo-EM images were collected on a 200 kV Talos Arctica transmission microscope (Thermo Fisher) equipped with a K3 direct electron detector (Gatan). The total exposure time of each movie stack was ˜3.5 s, leading to a total accumulated dose of 50 electrons per Å\hat{ }2 which fractionated into 50 frames. Dose fractionated super-resolution movie stacks collected from the K3 direct electron detector were 2× binned to a pixel size of 1.23 Å. The defocus value was set between −1.0 μm to −2.5 μm.

### Cryo-EM Data Processing

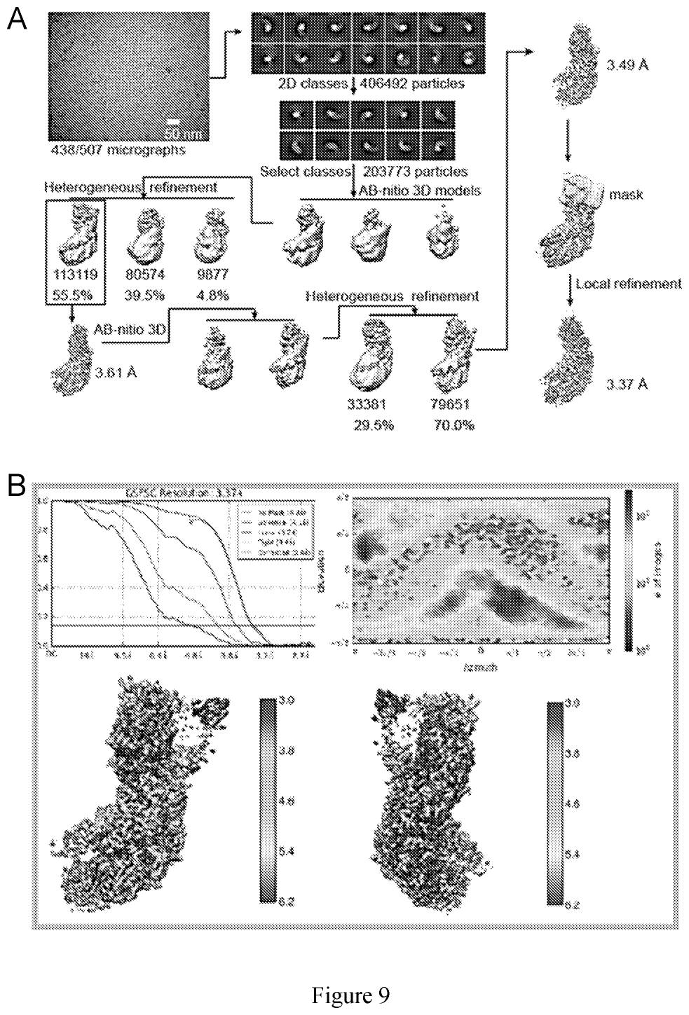

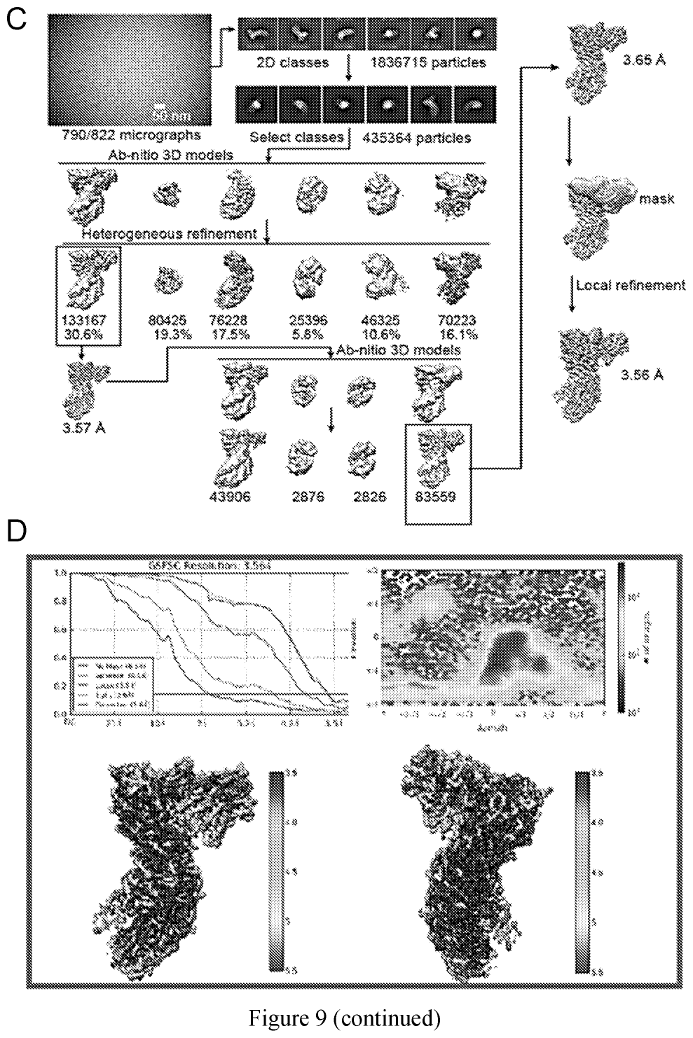

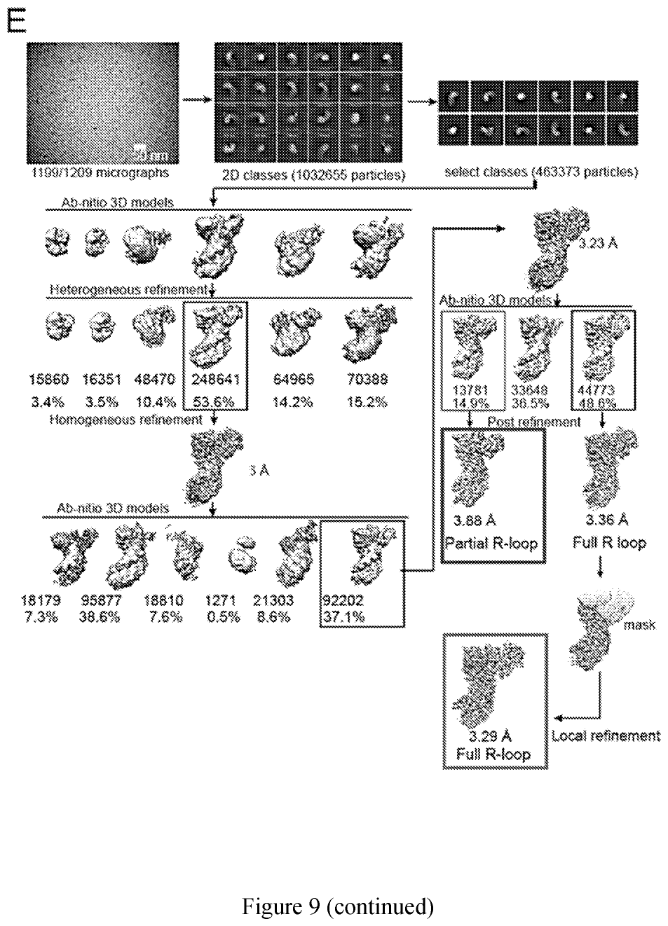

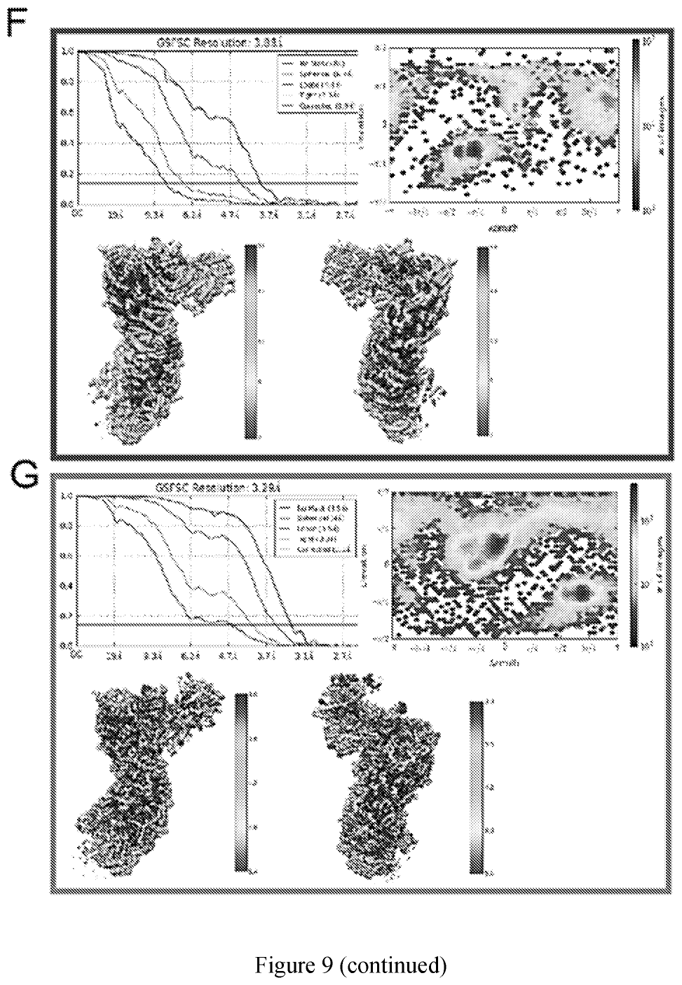

Motion correction, CTF-estimation, blob particle picking, 2D classification, 3D classification and non-uniform 3D refinement were performed in cryoSPARC v.2 (Punjani et al., 2017). Refinements followed the standard procedure, a series of 2D and 3D classifications with (1 symmetry were performed as shown in FIG. 9 to generate the final maps. A solvent mask was generated and was used for all subsequent local refinement steps. CTF post refinement was conducted to refine the beam-induced motion of the particle set, resulting in the final maps. The detailed data processing and refinement statistics for all cryo-EM structures are summarized in FIG. 9 and Table 1 for the data acquisition and structure refinement.

TABLES
Table 1. Cryo-EM data collection, refinement and validation statistics

| | Cascade | Cascade- | Partial | Full | Cas3-Cas8a |
|---|---|---|---|---|---|
| | alone | Cas3 | R-loop | R-loop | alone |
| Data collection | | | | | |
| Magnification(nominal) | 67k | 67k | 67k | 67k | 60k |
| Voltage (Kv) | 200 | 200 | 200 | 200 | 300 |
| Electron exposure(e−/Å²) | 50 | 50 | 50 | 50 | 60 |
| Defocus range(um) | 1.-2.5 | 1-2.5 | 1-2.5 | 1-2.5 | 0.8-2.5 |
| Raw Pixel size(Å) | 0.615 | 0.615 | 0.615 | 0.615 | 0.65 |
| symmetry | C1 | C1 | C1 | C1 | C1 |
| Micrographs | 507 | 822 | 1,209 | | 3,002 |
| Initial particle | 203,773 | 435,364 | 463,373 | | 601,678 |
| images(No.) | | | | | |
| Final particle images(No.) | 79,651 | 83,559 | 13,781 | 44,773 | 32,211 |
| Map resolution(Å) | 3.4 | 3.6 | 3.9 | 3.3 | 6.6 |
| FSC threshold | 0.143 | 0.143 | 0.143 | 0.143 | 0.143 |
| Map resolution range (Å) | 20-3.0 | 20-3.4 | 20-3.6 | 20-3.0 | 20-6.0 |
| Refinement | | | | | |
| Initial model used | De novo | De novo | De novo | De novo | |
| Map sharpening B | −100 | −100 | −100 | −100 | |
| factor(Å²) | | | | | |
| Model composition | | | | | |
| Nonhydrogen atoms | 27,450 | 33,583 | 34,173 | 34,874 | |
| Protein residues/ | 3,383/45 | 4,192/45 | 4,192/75 | 4,174/113 | |
| Nucleotide | | | | | |
| Ligands | 0 | 2 | 2 | 2 | |
| Water | 0 | 0 | 0 | 0 | |
| B factors (Å²) | | | | | |
| protein | 50.65/500.34/147.16 | 66.77/214.29/111.95 | 116.50/475.38/214.96 | 55.90/316.45/113.80 | |
| Nucleotide | 69.48/244.73/114.62 | 82.63/146.47/96.92 | 152.68/343.15/232.64 | 64.27/301.36/112.85 | |
| Ligand | — | 160.09/160.23/160.16 | 138.77/376.75/292.73 | 176.44/178.83/177.64 | |
| Water | — | — | — | — | |
| R.m.s.d deviations | | | | | |
| Bond lengths (Å) | 0.003(0) | 0.002(0) | 0.003(0) | 0.003(0) | |
| bond angles(°) | 0.574(4) | 0.549(9) | 0.643(10) | 0.607(7) | |
| Validation | | | | | |
| MolProbity score | 2.07 | 2.06 | 2.20 | 2.19 | |
| Clashscore | 13.36 | 11.68 | 15.77 | 15.97 | |
| Poor rotamers(%) | 0.00 | 0.00 | 0.00 | 0.00 | |
| Ramachandran plot | | | | | |
| Favored(%) | 93.33 | 92.13 | 91.60 | 92.10 | |
| Allowed(%) | 6.64 | 7.48 | 8.11 | 7.66 | |
| Disallowed(%) | 0.03 | 0.39 | 0.29 | 0.24 | |

References. This reference listing is not an indication that any reference is material to patentability:
Barrangou, R., Fremaux, C., Deveau, H., Richards, M., Boyaval, P., Moineau, S., Romero, D. A., and Horvath, P. (2007). CRISPR provides acquired resistance against viruses in prokaryotes. Science 315, 1709-1712.
Bolotin, A., Quinquis, B., Sorokin, A., and Ehrlich, S. D. (2005). Clustered regularly interspaced short palindrome repeats (CRISPRs) have spacers of extrachromosomal origin. Microbiology 151, 2551-2561.
Brouns, S. J., Jore, M. M., Lundgren, M., Westra, E. R., Slijkhuis, R. J., Snijders, A. P., Dickman, M. J., Makarova, K. S., Koonin, E. V., and van der Oost, J. (2008). Small CRISPR RNAs guide antiviral defense in prokaryotes. Science 321, 960-964.
Caliando, B. J., and Voigt, C. A. (2015). Targeted DNA degradation using a CRISPR device stably carried in the host genome. Nat Commun 6, 6989.
Cameron, P., Coons, M. M., Klompe, S. E., Lied, A. M., Smith, S. C., Vidal, B., Donohoue, P. D., Rotstein, T., Kohrs, B. W., Nyer, D. B., et al. (2019). Harnessing type I CRISPR-Cas systems for genome engineering in human cells. Nat Biotechnol 37, 1471-1477.
Carte, J., Wang, R., Li, H., Terns, R. M., and Terns, M. P. (2008). Cas6 is an endoribonuclease that generates guide RNAs for invader defense in prokaryotes. Genes Dev 22, 3489-3496.
Chen, J. S., Ma, E., Harrington, L. B., Da Costa, M., Tian, X., Palefsky, J. M., and Doudna, J. A. (2018). CRISPR-Cas12a target binding unleashes indiscriminate single-stranded DNase activity. Science 360, 436-439.
Chen, Y., Liu, J., Zhi, S., Zheng, Q., Ma, W., Huang, J., Liu, Y., Liu, D., Liang, P., and Songyang, Z. (2020). Repurposing type I-F CRISPR-Cas system as a transcriptional activation tool in human cells. Nat Commun 11, 3136.
Csorgo, B., Leon, L. M., Chau-Ly, I. J., Vasquez-Rifo, A., Berry, J. D., Mahendra, C., Crawford, E. D., Lewis, J. D., and Bondy-Denomy, J. (2020). A compact Cascade-Cas3 system for targeted genome engineering. Nat Methods 17, 1183-1190.
Dillard, K. E., Brown, M. W., Johnson, N. V., Xiao, Y., Dolan, A., Hernandez, E., Dahlhauser, S. D., Kim, Y., Myler, L. R., Anslyn, E. V., et al. (2018). Assembly and Translocation of a CRISPR-Cas Primed Acquisition Complex. Cell.
Dolan, A. E., Hou, Z., Xiao, Y., Gramelspacher, M. J., Heo, J., Howden, S. E., Freddolino, P. L., Ke, A., and Zhang, Y. (2019). Introducing a Spectrum of Long-Range Genomic Deletions in Human Embryonic Stem Cells Using Type I CRISPR-Cas. Mol Cell.
Elmore, J., Deighan, T., Westpheling, J., Terns, R. M., and Terns, M. P. (2015). DNA targeting by the type I-G and type I-A CRISPR-Cas systems of Pyrococcus furiosus. Nucleic Acids Res 43, 10353-10363.
Elmore, J. R., Sheppard, N. F., Ramia, N., Deighan, T., Li, H., Terns, R. M., and Terns, M. P. (2016). Bipartite recognition of target RNAs activates DNA cleavage by the Type III-B CRISPR-Cas system. Genes Dev 30, 447-459.
Foster, K., Gruschow, S., Bailey, S., White, M. F., and Terns, M. P. (2020). Regulation of the RNA and DNA nuclease activities required for Pyrococcus furiosus Type III-B CRISPR-Cas immunity. Nucleic Acids Res 48, 4418-4434.
Garrett, S., Shiimori, M., Watts, E. A., Clark, L., Graveley, B. R., and Terns, M. P. (2020). Primed CRISPR DNA uptake in Pyrococcus furiosus. Nucleic Acids Res 48, 6120-6135.
Gong, B., Shin, M., Sun, J., Jung, C. H., Bolt, E. L., van der Oost, J., and Kim, J. S. (2014). Molecular insights into DNA interference by CRISPR-associated nuclease-helicase Cas3. Proc Natl Acad Sci USA 111, 16359-16364.
Gootenberg, J. S., Abudayyeh, O. O., Kellner, M. J., Joung, J., Collins, J. J., and Zhang, F. (2018). Multiplexed and portable nucleic acid detection platform with Cas13, Cas12a, and Csm6. Science 360, 439-444.
Govindarajan, S., Borges, A., and Bondy-Denomy, J. (2020). Distinct subcellular localization of a Type I CRISPR complex and the Cas3 nuclease in bacteria. bioRxiv, 2020.2009.2029.318501.
Guo, T. W., Bartesaghi, A., Yang, H., Falconieri, V., Rao, P., Merk, A., Eng, E. T., Raczkowski, A. M., Fox, T., Earl, L. A., et al. (2017). Cryo-EM Structures Reveal Mechanism and Inhibition of DNA Targeting by a CRISPR-Cas Surveillance Complex. Cell 171, 414-426 e412.
Hayes, R. P., Xiao, Y., Ding, F., van Erp, P. B., Rajashankar, K., Bailey, S., Wiedenheft, B., and Ke, A. (2016). Structural basis for promiscuous PAM recognition in type I-E Cascade from E. coli. Nature 530, 499-503.
He, L., Matosevic, Z. J., Mitic, D., Markulin, D., Killelea, T., Matkovic, M., Bertosa, B., Ivancic-Bace, I., and Bolt, E. L. (2021). A Tryptophan ‘Gate’ in the CRISPR-Cas3 Nuclease Controls ssDNA Entry into the Nuclease Site, That When Removed Results in Nuclease Hyperactivity. Int J Mol Sci 22.
Hochstrasser, M. L., Taylor, D. W., Bhat, P., Guegler, C. K., Sternberg, S. H., Nogales, E., and Doudna, J. A. (2014). CasA mediates Cas3-catalyzed target degradation during CRISPR RNA-guided interference. Proc Natl Acad Sci US A 111, 6618-6623.
Hochstrasser, M. L., Taylor, D. W., Kornfeld, J. E., Nogales, E., and Doudna, J. A. (2016). DNA Targeting by a Minimal CRISPR RNA-Guided Cascade. Mol Cell 63, 840-851.
Huo, Y., Nam, K. H., Ding, F., Lee, H., Wu, L., Xiao, Y., Farchione, M. D., Jr., Zhou, S., Rajashankar, K., Kurinov, I., et al. (2014). Structures of CRISPR Cas3 offer mechanistic insights into Cascade-activated DNA unwinding and degradation. Nat Struct Mol Biol 21, 771-777.
Jackson, R. N., Golden, S. M., van Erp, P. B., Carter, J., Westra, E. R., Brouns, S. J., van der Oost, J., Terwilliger, T. C., Read, R. J., and Wiedenheft, B. (2014). Crystal structure of the CRISPR RNA-guided surveillance complex from Escherichia coli. Science 345, 1473-1479.
Jia, N., Mo, C. Y., Wang, C., Eng, E. T., Marraffini, L. A., and Patel, D. J. (2019). Type III-A CRISPR-Cas Csm Complexes: Assembly, Periodic RNA Cleavage, DNase Activity Regulation, and Autoimmunity. Mol Cell 73, 264-277 e265.
Kunne, T., Kieper, S. N., Bannenberg, J. W., Vogel, A. I., Miellet, W. R., Klein, M., Depken, M., Suarez-Diez, M., and Brouns, S. J. (2016). Cas3-Derived Target DNA Degradation Fragments Fuel Primed CRISPR Adaptation. Mol Cell 63, 852-864.
Li, Y., Pan, S., Zhang, Y., Ren, M., Feng, M., Peng, N., Chen, L., Liang, Y. X., and She, Q. (2016). Harnessing Type I and Type III CRISPR-Cas systems for genome editing. Nucleic Acids Res 44, e34.
Lin, J., Fuglsang, A., Kjeldsen, A. L., Sun, K., Bhoobalan-Chitty, Y., and Peng, X. (2020). DNA targeting by subtype I-D CRISPR-Cas shows type I and type III features. Nucleic Acids Res 48, 10470-10478.
Luo, M. L., Mullis, A. S., Leenay, R. T., and Beisel, C. L. (2015). Repurposing endogenous type I CRISPR-Cas systems for programmable gene repression. Nucleic Acids Res 43, 674-681
Majumdar, S., and Terns, M. P. (2019). CRISPR RNA-guided DNA cleavage by reconstituted Type I-A immune effector complexes. Extremophiles 23, 19-33.
Majumdar, S., Zhao, P., Pfister, N. T., Compton, M., Olson, S., Glover, C. V., 3rd, Wells, L., Graveley, B. R., Terns, R. M., and Terns, M. P. (2015). Three CRISPR-Cas immune effector complexes coexist in Pyrococcus furiosus. RNA 21, 1147-1158.
Makarova, K. S., Grishin, N. V., Shabalina, S. A., Wolf, Y. I., and Koonin, E. V. (2006). A putative RNA-interference-based immune system in prokaryotes: computational analysis of the predicted enzymatic machinery, functional analogies with eukaryotic RNAi, and hypothetical mechanisms of action. Biol Direct 1, 7.
Makarova, K. S., Wolf, Y. I., Iranzo, J., Shmakov, S. A., Alkhnbashi, O. S., Brouns, S. J. J., Charpentier, E., Cheng, D., Haft, D. H., Horvath, P., et al. (2020). Evolutionary classification of CRISPR-Cas systems: a burst of class 2 and derived variants. Nature reviews Microbiology 18, 67-83.
Marraffini, L. A., and Sontheimer, E. J. (2008). CRISPR interference limits horizontal gene transfer in staphylococci by targeting DNA. Science 322, 1843-1845.
McBride, T. M., Schwartz, E. A., Kumar, A., Taylor, D. W., Fineran, P. C., and Fagerlund, R. D. (2020). Diverse CRISPR-Cas Complexes Require Independent Translation of Small and Large Subunits from a Single Gene. Mol Cell 80, 971-979 e977.
Mojica, F. J., García-Martínez, J., and Soria, E. (2005). Intervening sequences of regularly spaced prokaryotic repeats derive from foreign genetic elements. Journal of molecular evolution 60, 174-182.
Morisaka, H., Yoshimi, K., Okuzaki, Y., Gee, P., Kunihiro, Y., Sonpho, E., Xu, H., Sasakawa, N., Naito, Y., Nakada, S., et al. (2019). CRISPR-Cas3 induces broad and unidirectional genome editing in human cells. Nat Commun 10, 5302.
Mulepati, S., and Bailey, S. (2013). In vitro reconstitution of an Escherichia coli RNA-guided immune system reveals unidirectional, ATP-dependent degradation of DNA target. J Biol Chem 288, 22184-22192.
Mulepati, S., Heroux, A., and Bailey, S. (2014). Crystal structure of a CRISPR RNA-guided surveillance complex bound to a ssDNA target. Science 345, 1479-1484.
Notomi, T., Okayama, H., Masubuchi, H., Yonekawa, T., Watanabe, K., Amino, N., and Hase, T. (2000). Loop-mediated isothermal amplification of DNA. Nucleic Acids Res 28, E63.
O'Brien, R. E., Santos, I. C., Wrapp, D., Bravo, J. P. K., Schwartz, E. A., Brodbelt, J. S., and Taylor, D. W. (2020). Structural basis for assembly of non-canonical small subunits into type I-C Cascade. Nat Commun 11, 5931.
Osakabe, K., Wada, N., Miyaji, T., Murakami, E., Marui, K., Ueta, R., Hashimoto, R., Abe-Hara, C., Kong, B., Yano, K., et al. (2020). Genome editing in plants using CRISPR type I-D nuclease. Commun Biol 3, 648.
Osakabe, K., Wada, N., Murakami, E., Miyashita, N., and Osakabe, Y. (2021). Genome editing in mammalian cells using the CRISPR type I-D nuclease. Nucleic Acids Res.
Pickar-Oliver, A., Black, J. B., Lewis, M. M., Mutchnick, K. J., Klann, T. S., Gilcrest, K. A., Sitton, M. J., Nelson, C. E., Barrera, A., Bartelt, L. C., et al. (2019). Targeted transcriptional modulation with type I CRISPR-Cas systems in human cells. Nat Biotechnol 37, 1493-1501.
Plagens, A., Tripp, V., Daume, M., Sharma, K., Klingl, A., Hrle, A., Conti, E., Urlaub, H., and Randau, L. (2014). In vitro assembly and activity of an archaeal CRISPR-Cas type I-A Cascade interference complex. Nucleic Acids Res 42, 5125-5138.
Pourcel, C., Salvignol, G., and Vergnaud, G. (2005). CRISPR elements in Yersinia pestis acquire new repeats by preferential uptake of bacteriophage DNA, and provide additional tools for evolutionary studies. Microbiology 151, 653-663
Punjani, A., Rubinstein, J. L., Fleet, D. J., and Brubaker, M. A. (2017). cryoSPARC: algorithms for rapid unsupervised cryo-EM structure determination. Nat Methods 14, 290-296.
Rath, D., Amlinger, L., Hoekzema, M., Devulapally, P. R., and Lundgren, M. (2015). Efficient programmable gene silencing by Cascade. Nucleic Acids Res 43, 237-246.
Redding, S., Sternberg, S. H., Marshall, M., Gibb, B., Bhat, P., Guegler, C. K., Wiedenheft, B., Doudna, J. A., and Greene, E. C. (2015). Surveillance and Processing of Foreign DNA by the Escherichia coli CRISPR-Cas System. Cell 163, 854-865.
Rollins, M. F., Chowdhury, S., Carter, J., Golden, S. M., Miettinen, H. M., Santiago-Frangos, A., Faith, D., Lawrence, C. M., Lander, G. C., and Wiedenheft, B. (2019). Structure Reveals a Mechanism of CRISPR-RNA-Guided Nuclease Recruitment and Anti-CRISPR Viral Mimicry. Mol Cell 74, 132-142 e135.
Rutkauskas, M., Sinkunas, T., Songailiene, I., Tikhomirova, M. S., Siksnys, V., and Seidel, R. (2015). Directional R-Loop Formation by the CRISPR-Cas Surveillance Complex Cascade Provides Efficient Off-Target Site Rejection. Cell reports.
Sashital, D. G., Wiedenheft, B., and Doudna, J. A. (2012). Mechanism of foreign DNA selection in a bacterial adaptive immune system. Mol Cell 46, 606-615.
Sinkunas, T., Gasiunas, G., Waghmare, S. P., Dickman, M. J., Barrangou, R., Horvath, P., and Siksnys, V. (2013). In vitro reconstitution of Cascade-mediated CRISPR immunity in Streptococcus thermophilus. EMBO J 32, 385-394.
Szczelkun, M. D., Tikhomirova, M. S., Sinkunas, T., Gasiunas, G., Karvelis, T., Pschera, P., Siksnys, V., and Seidel, R. (2014). Direct observation of R-loop formation by single RNA-guided Cas9 and Cascade effector complexes. Proc Natl Acad Sci US A 111, 9798-9803.
Tan, R., Krueger, R. K., Gramelspacher, M. J., Zhou, X., Xiao, Y., Ke, A., Hou, Z., and Zhang, Y. (2022). Cas11 enables genome engineering in human cells with compact CRISPR-Cas3 systems. Mol Cell.
Wang, L., Mo, C. Y., Wasserman, M. R., Rostol, J. T., Marraffini, L. A., and Liu, S. (2019). Dynamics of Cas10 Govern Discrimination between Self and Non-self in Type III CRISPR-Cas Immunity. Mol Cell 73, 278-290 e274.
Westra, E. R., van Erp, P. B., Kunne, T., Wong, S. P., Staals, R. H., Seegers, C. L., Bollen, S., Jore, M. M., Semenova, E., Severinov, K., et al. (2012). CRISPR immunity relies on the consecutive binding and degradation of negatively supercoiled invader DNA by Cascade and Cas3. Mol Cell 46, 595-605.
Wiedenheft, B., Lander, G. C., Zhou, K., Jore, M. M., Brouns, S. J. J., van der Oost, J., Doudna, J. A., and Nogales, E. (2011). Structures of the RNA-guided surveillance complex from a bacterial immune system. Nature 477, 486-489.
Xiao, Y., Luo, M., Dolan, A. E., Liao, M., and Ke, A. (2018). Structure basis for RNA-guided DNA degradation by Cascade and Cas3. Science 361.
Xiao, Y., Luo, M., Hayes, R. P., Kim, J., Ng, S., Ding, F., Liao, M., and Ke, A. (2017). Structure Basis for Directional R-loop Formation and Substrate Handover Mechanisms in Type I CRISPR-Cas System. Cell 170, 48-60 ell.
Yoshimi, K., Takeshita, K., Kodera, N., Shibumura, S., Yamauchi, Y., Omatsu, M., Kunihiro, Y., Yamamoto, M., and Mashimo, T. (2021). Dynamic mechanisms of CRISPR interference by <em>Escherichia coli</em> CRISPR-Cas3. bioRxiv, 2021.2007.2018.452824.
You, L., Ma, J., Wang, J., Artamonova, D., Wang, M., Liu, L., Xiang, H., Severinov, K., Zhang, X., and Wang, Y. (2020). Structure Studies of the CRISPR-Csm Complex Reveal Mechanism of Co-transcriptional Interference. Cell 176, 239-253 e216.
Young, J. K., Gasior, S. L., Jones, S., Wang, L., Navarro, P., Vickroy, B., and Barrangou, R. (2019). The repurposing of type I-E CRISPR-Cascade for gene activation in plants. Commun Biol 2, 383.
Zhao, H., Sheng, G., Wang, J., Wang, M., Bunkoczi, G., Gong, W., Wei, Z., and Wang, Y. (2014). Crystal structure of the RNA-guided immune surveillance Cascade complex in Escherichia coli. Nature 515, 147-150.

### Supplementary Tables

SUPPLEMENTARY TABLE 1
Plasmids used in this work.

| | | Derived from | | |
|---|---|---|---|---|
| Name in this study | Insert | vector | Marker | purpose |
| pCDFuet-Cascade/Cas3 | Whole gene cluster | pETDeut | streptomycin | For |
| | (pCascade/Cas3) | | | interference |
| | | | | assay |
| pCRISPR-1-ribozymes | CRISPR array | pRSFDuet | Kanamycin | purification |
| pCRISPR-1-PfuCas6a | CRISPR array | pRSFDuet | Kanamycin | purification |
| pCRISPR-1-EcoCas6e | CRISPR array | pRSFDuet | Kanamycin | purification |
| pETDuet-Target DNA | Target DNA | pRSFDuet | Ampicilin | interference |
| | | | | assay |
| pCRISPR-GFP_G1- | CRISPR array | pRSFDuet | Kanamycin | purification |
| EcoCas6e | | | | |
| pCRISPR-GFP G2- | CRISPR array | pRSFDuet | Kanamycin | purification |
| EcoCas6e | | | | |
| pRSFDuet-G1 target DNA | Target DNA | pRSFDuet | Kanamycin | Cleavage |
| | | | | assay |
| pETDuet-Cas11-Cas7- | Cas11-Cas7-Cas5 | pETDuet | Ampicilin | purification |
| Cas5-His tag | | | | |
| pCDFDuet-Strep tag-Cas8 | Cas8 | pCDFDuet | streptomycin | purification |
| pRSFDuet-Cas3HD | Cas3HD | pRSFDuet | Kanamycin | purification |
| pRSFDuet-Cas3HEL | Cas3HEL | pRSFDuet | Kanamycin | purification |
| pRSFDuet-Cas3HEL-NLS | Cas3HEL | pRSFDuet | Kanamycin | purification |
| tag | | | | |
| pRSFDuet-Cas3HD-NLS | Cas3HD | pRSFDuet | Kanamycin | purification |
| tag | | | | |
| pCDFDuet-Strep tag-NLS | Cas8 | pCDFDuet | streptomycin | purification |
| tag-Cas8 | | | | |
| pETDuet-Cas11-Cas7-NLS | Cas11-Cas7-Cas5 | pETDuet | Ampicilin | purification |
| tag-Cas5-His | | | | |
| pET19-Strep tag-NLS tag- | CasA | Mol cell, 2018 | Ampicilin | purification |
| TfuCasA-NLS | | | | |
| pCDFDuet-TfuCasB-CasC- | CasB-E | Mol cell, 2018 | streptomycin | purification |
| NLS-CasD-CasE | | | | |
| pET19-TfuCas3-NLS-Strep | Cas3 | Mol cell, 2018 | Ampicilin | purification |
| tag | | | | |
| pJET-I-A G1 deletion | deletion library | pJET | Kanamycin | Sequencing |
| library | | | | |
| pETDuet-Cas11-Cas7- | Cas11-Cas7-Cas5 | pETDuet | Ampicilin | purification |
| Cas5-His tag | | | | |
| pCDFDuet-Target DNA | Target DNA | pRSFDuet | streptomycin | interference |
| | | | | assay |

SUPPLEMENTARY TABLE 2
Primers and protein sequence used in this work.

| SEQ | | | |
|---|---|---|---|
| ID NO: | Name | Sequence (5′-3′) | Description |
| 17 | CY032 | ATATGGATCCATGGGGGGATGGATCAGAAATATTG | PfuCas11a-Cas7a- |
| | | | Cas5a PCR |
| | | | amplification |
| 18 | CY033 | ATATCTCGAGTCAGCTCTCTGAAGAGTTTTTCGG | PfuCas11a-Cas7a- |
| | | | Cas5a PCR |
| | | | amplification |
| 19 | CY034 | ATATGGATCC ATGAAATTTAACGAATTTAAAACACCT | PfuCas8a PCR |
| | | | amplification |
| 20 | CY035 | ATATCTCGAGTTAACGTTCACATATTATATCCTCC | PfuCas8a PCR |
| | | | amplification |
| 21 | CY030 | ATATGGATCCATGAGGTTTTTAATAAGACTAGTTCC, | PfuCas6a PCR |
| | | | amplification |
| 22 | CY031 | ATATCTCGAGTTATACTCCAGTTTTTAATTCCTCTC | PfuCas6a PCR |
| | | | amplification |
| 23 | CY077 | GATTCAGGATCCATGTATCTCAGTAAAGTCATCATTG, | EcoCas6e PCR |
| | | | amplification |
| 24 | CY078 | GTAGTTCTCGAGTCACAGTGGAGCCAAAGATAGC | EcoCas6e PCR |
| | | | amplification |
| 25 | CY042 | GTTCCAATAAGACTACAAAAGAATTGAAAGAGTGCTTCC | Pfu repeat-spacer- |
| | | CCAAACCCTTAACTGGTTGTAACAGTTGGTTCCAATAAGA | repeat For pfuCas6a |
| | | CTACAAAAG (pcrRNA) | processing |
| 26 | CY043 | CAACTTTCAATTGATGAGTCCGTGAGGACGAAACGGTAC | HH ribozyme-Pfu 5′ |
| | | CCGGTACCGTCATTGAAAGTTG [symbol] | handle-spacer-HP |
| | | [symbol] AGAGTTCC | ribozyme in |
| | | CCGCGCCAGCGGGGATAAACCGtggcagtcctgttgaaaaac | lowercase |
| | | agagaagccaaccagagaaacacacgttgtggtatatt, | |
| | | acctggtaagctatctaa | |
| | | (pcrRNA) | |
| 27 | CY072 | GAGTTCCCCGCGCCAGCGGGGATTGAAAG [symbol] | Eco repeat |
| | | [symbol] | (recognition and |
| | | GAGTTCCCCGCGCCAGCGGGG , (pcrRNA) | cleavage part)-Pfu |
| | | | 5′ handle-spacer- |
| | | | Eco repeat, For |
| | | | EcoCas6e processing |
| 28 | CY056 | GCCGTACCTCTGAATCACGAAGGGGTTTGGGAATTGACC | Target DNA, CCC |
| | | AACATTGTCAACGGGAGTTACTCGCG (TS), | PAM-spacer |
| 29 | CY057 | CGCGAGTAACTCCCAGTGCTTCCCCAAACCCTTAACTGGT | Target DNA, CCC |
| | | TGTAACAGTTGTTCAGAGGTACGGC (NTS) | PAM-spacer |
| 30 | CY216 | GCCGTACCTCTGAATCACGAAGGGGTTTGGGAATTGACC | Target DNA, TGC |
| | | AACATTGTCAAC [symbol] AGTTACTCGCG (TS), | PAM-spacer |
| 31 | CY217 | CGCGAGTAACT [symbol] AGTGCTTCCCCAAACCCTTAACTG | Target DNA, TGC |
| | | GTTGTAACAGTTGTTCAGAGGTACGGC (NTS) | PAM-spacer |
| 32 | CY218 | GCCGTACCTCTGAATCACGAAGGGGTTTGGGAATTGACC | Target DNA, GCA |
| | | AACATTGTCAAC [symbol] AGTTACTCGCG (TS), | PAM-spacer |
| 33 | CY219 | CGCGAGTAACT [symbol] AGTGCTTCCCCAAACCCTTAACTG | Target DNA, GCA |
| | | GTTGTAACAGTTGTTCAGAGGTACGGC (NTS) | PAM-spacer |
| 34 | CY179 | ATATAT GGATCC AGTTGTAAGGCATTCCAAGGA, | Pfu Cas3 HD PCR |
| | | | amplification |
| 35 | CY037 | GATATGTCGACTCACTTCATCATTTCAACTCTAAGA | Pfu Cas3 HD PCR |
| | | | amplification |
| 36 | CY180 | ATATAT GTCGAC TCATACTCTCACCCCCCAGT, | Pfu Cas3 HEL PCR |
| | | | amplification |
| 37 | CY036 | ATATGGATCCATGGATACCGAAAAACTCTTCAGAG | Pfu Cas3 HEL PCR |
| | | | amplification |
| 38 | CY057 | /56-FAM/- | F-ssDNA reporter |
| | | CGCGAGTAACTCCCTTGTAGTATGCGGTCCTTGCGGCTG | |
| | | AGAGCACTTCAGAGGTACGGC | |
| 39 | CY579 | CCCAGAGCAGGGCCTTAGGGAAG, | 1 kb Forward and |
| | | | reverse primers to |
| | | | map genome |
| | | | deletions. |
| 40 | CY581 | GAAAGATGGCCGCTTCCCG | 1 kb Forward and |
| | | | reverse primers to |
| | | | map genome |
| | | | deletions. |
| 41 | | CAGACCCAGGAGTCCAGGC, | 2 kb Forward and |
| | | | reverse primer for |
| | | | mapping the genome |
| | | | editing. |
| 42 | | GCTGGGACCACCTTATATTCCC | 2 kb Forward and |
| | | | reverse primer for |
| | | | mapping the genome |
| | | | editing. |
| 43 | CY584 | AGGGATCCTGTGTGGCCATC, | 2 kb Forward and |
| | | | reverse primer for |
| | | | mapping the genome |
| | | | editing. |
| 44 | CY585 | TGCGATGTCCGGAGAGGATGG | 2 kb Forward and |
| | | | reverse primer for |
| | | | mapping the genome |
| | | | editing. |
| 45 | SSDNA_FQ | /56-FAM/-AAAAAAAA/3BHQ1 | ssDNA-FQ reporter |
| 46 | SSDNA_F_ | /56FAM/-AGGGAAACCTTTGGGCCCAATTCCGG/3Biotin/ | ssDNA-F-Biotin |
| | Biotin | | reporter |
| 1 | Cas8a | MGSSWSHPQFEKGGGSGGGSGGSAWSHPQFEKMSDSEV | Twin-strep tag- |
| | sequence | NQEAKPEVKPEVHREQIGGSKFNEFKTPQIDPIFDLYVAYGY | linker-Cas8a |
| | | VESLIRGGAKEATLIPHGASYLIQTDVSNEEFRHGLVDALSE | |
| | | MLSLHIALARHSPREGGKLVSDADFSAGANINNVYWDSVPRN | |
| | | LEKVMKDLEKKRSVKGTATIPITLMPSAGKYMLKHFGVQGGN | |
| | | PIKVDLLNYALAWVGFHYYTPYIKYAKGDTTWIHIYQIAPV | |
| | | EEVDMISILSLKDLKMHLPHYYESNLDFLINRRLALLYHLLHS | |
| | | ESLGALELFTEKEFVIHSYTLERSGNNQAIRSFEEEEIGKLMD | |
| | | FLWKLKRRDFYHAIKFIDDLLKKATEGALALIDAIMNERLEGF | |
| | | YTALKLGKKAGVVSSREIVAALEDIICER. | |
| 2 | Cas11a | MGGWIRNIGRYLSYLVDDTFEEYAYDVVDGIAKARTQEELLE | no tag |
| | sequence | GVYKALRLAPKLKKKAESKGCPPPRIPSPEDIEALEEKVEQL | |
| | | SNPKDLRKLAVSLALWAFASWNNCPKKGKGTEGGVE | |
| 3 | Cas7a | MMYVRISGRIRLNAHSLNAQGGGGTNYIEITKTKVTVRTENG | no tag |
| | sequence | WTVVEVPAITGNMLKHWHFVGFVDYFKTTPYGVNLTERALR | |
| | | YNGTRFGQGETTATKANGATVQLNDEATIIKELADADVHGFL | |
| | | APKTGRRRVSLVKASFILPTEDFIKEVEGERLITAIKHNRVDV | |
| | | DEKGAIGSSKEGTAQMLFSREYATGLYGFSIVLDLGLVGIPQ | |
| | | GLPVKFEENQPRPNIVIDPNERKARIESALKALIPMLSGYIGA | |
| | | NLARSFPVFKVEELVAIASEGPIPALVHGFYEDYIEANRSIIK | |
| | | NARALGFNIEVFTYNVDLGEDIEATKVSSVEELVANLVKMVGG | |
| | | KE | |
| 4 | Cas5a | MDILLVCLRFPFFSVAKRSYQVRTSFLLPPPSALKGALAKGLI | His-tag |
| | sequence | LLKPEKYASSSLDEAALKAIKEIESKLVDIKAVSVAPLSPLIR | |
| | | NAFLLKRLRNLESGSNAEKSDAMRREYTFTRELLVAYIFKNLT | |
| | | QEEKNLYLKAAMLIDVIGDTESLATPVWASFVKPEDKKAPLAF | |
| | | SAPYTEIYSLLSSKIQAKGKIRMYIEKMRVSPEYSKTKGPQEE | |
| | | IFYLPIEERRYKRIVYYARTIYPPEVEKALTVDGEVLGIWIPK | |
| | | NSSESGSHHHHHHH | |
| 5 | Cas6e for | MYLSKVIIARAWSRDLYQLHQGLWHLFPNRPDAARDFLFHV | no tag |
| | crRNA | EKRNTPEGCHVLLQSAQMPVSTAVATVIKTKQVEFQLQVGV | |
| | processing | PLYFRLRANPIKTILDNQKRLDSKGNIKRCRVPLIKEAEQI | |
| | | AWLQRKLGNAARVEDVHPISERPQYFSGDGKSGKIQTVCFE | |
| | | GVLTINDAPALIDLVQQGIGPAKSMGCGLLSLAPL | |
| 6 | Cas3_HEL | MGSSHHHHHHSQLEVLFQGPLDTEKLFRELTGFEPYDYQLR | His tag-3C site- |
| | | AWEKIREIMNNGGKVIIEVPTAGGKTETAVMPFFAGIYNNNW | Cas3HEL |
| | | PVARLVYVLPTRSLVEKQAERLRNLVYKLLQLKGKSKEEAEK | |
| | | LARELVVVEYGLEKTHAFLGWVVVTTWDAFLYGLAAHRTVG | |
| | | NRFTFPAGAIAQSLVIFDEVQMYQDESMYMPRLLSLVVGILE | |
| | | EANVPLVIMSATIPSKLREMIAGDTEVITVDKNDKNKPSRGNV | |
| | | KVRLVEGDITDVLNDIKKILKNGKKVLVVRNTVRKAVETYQVL | |
| | | KKKLNDTLANPSDALLIHSRFTIGDRREKERALDSARLIVATQ | |
| | | VVEAGLDLPNVGLVVTDIAPLDALIQRIGRCARRPGEEGEGII | |
| | | LIPVENCIEHEKIVRGLSELMEKIGEDTVVFATVTSTNEYDRV | |
| | | VEIHYGEGKKNFVYVGDIDTARRVLEKKRSKKLPKDLYIIPYS | |
| | | VSPYDPLVLLTTYDELSKIGEYLADTTKARKALDRVYKFHYE | |
| | | NNIVPKEFASAYIYFKELKLFSAPPEYELRSRPELYVLLYPMNI | |
| | | EKNERVEDKVIDNLETARIIRISYSVKEWKKSDVVIGRLMKEW | |
| | | DKNAEKWVWKVRKSFKIDPYEIYVIDAKYYNSELGFITNLSDT | |
| | | NSHTDSDSKVRTRNSEHSSKKNRSKGKKGQTSLENWGVRV | |
| 7 | Cas3_HD | MGSSHHHHHHSQLEVLFQGPLGSSCKAFQGQTLREHIEAML | His tag-3C site- |
| | | AAWEIVKNKYIPSIIRVMKTVGVKFTEEDADKFMKTLIILHD | Cas3HD |
| | | VGKCSEVYQKHLSNNEPLRGFRHELVSAYYAYNILKDMFKDE | |
| | | TIAFIGALVVMMHHEPILMGQIRSLDKEELTPEVVLDKLRTFN | |
| | | GVMEGTESFIKSMIKEKLGVIPKVPSPTQEDVLREVIRLSVLA | |
| | | RHRPDSGKLRMVVGALLIPLVLCDYKGAKEREGESPKFAEV | |
| | | LRVEMMK | |

SUPPLEMENTARY TABLE 3
Miniature CRISPR array spacers
used in this study.

| SEQ | | |
|---|---|---|
| ID | Spacer | |
| NO: | Target | Sequence (5′-3′) |
| 47 | GFP_G1 | TGAGCAAAGACCCCAACGA |
| | | GAAGCGCGATCACATGGT |
| 48 | GFP_G2 | AGGATGTTGCCGTCCTCCT |
| | | TGAAGTCGATGCCCTTCA |
| 49 | Ribosomal | CATCTCTTTACAGGACCCT |
| | protein L3 | GGGTTCCCTTACCCTTTG |
| 50 | FtsZ | TGTCAATTCCTTCCCAATT |
| | | AGTATCTTCTGGTGGGCC |
| 51 | Valine tRNA | CAGTTATATGTGGAACGAA |
| | ligase | GGGTGCTAGGAGAAGCAT |

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A recombinantly produced or isolated Type I-A clustered regularly interspaced short palindromic repeats (CRISPR) system for use in DNA modification, wherein the system comprises:
i) a Cas8a-1 protein;
ii) a Cas3 complex comprising Cas3′ and Cas3″;
iii) a Cas5a protein;
iv) a Cas7a protein;
vi) a Cas11a protein;
wherein at least one of said proteins of i)-vi) comprises an amino acid sequence that is at least 85% identical to said protein that is encoded by a Type I-A CRISPR system, said system optionally being Pyrococcus furiosus (P. furiosus); and
vi) a heterologous Cas6 protein that is not encoded by P. furiosus.

2. The Type I-A CRISPR system of claim 1, wherein at least one of said proteins is modified to comprise a nuclear localization signal, an amino acid linker sequence, a protein purification tag, a detectable label, or a combination thereof.

3. The Type I-A CRISPR system of claim 1, further comprising a chimeric guide RNA comprising a P. furiosus encoded 5′-handle sequence, and a 3′-handle sequence that is not encoded by P. furiosus, said chimeric guide RNA being functional with the system of claim 1, and wherein optionally, a precursor RNA to the chimeric guide RNA is not produced using P. furiosus.

4. The Type I-A CRISPR system of claim 3, wherein the guide RNA is directed to a target sequence that is comprised by a chromosome or an extrachromosomal element.

5. The Type I-A CRISPR system of claim 4, wherein the guide RNA is directed to a target sequence that is comprised by the chromosome.

6. The type I-A CRISPR system of claim 4, wherein the target sequence is linked to a cancer associated gene.

7. The type I-A CRISPR system of claim 4, wherein the target sequence is linked to an inherited genetic disease associated gene.

8. The type I-A CRISPR system of claim 4, wherein the target sequence is linked to an integrated viral genomic sequence.

9. The type I-A CRISPR system of claim 4, wherein the guide RNA is directed to a target sequence that is comprised by the extrachromosomal element.

10. The type I-A CRISPR system of claim 7, wherein the sequence that is comprised by the extrachromosomal element comprises a viral genomic sequence.

11. The type I-A CRISPR system of claim 7, wherein the sequence that is comprised by the extrachromosomal circular DNA comprises a genomic sequence.

12. One or more expression vectors encoding the Type I-A CRISPR system of any one of claims 1-11.

13. A method for modifying double stranded DNA within cells, the method comprising introducing into the cell the a recombinantly produced or isolated Type I-A clustered regularly interspaced short palindromic repeats (CRISPR) system, wherein the system comprises:
i) a Cas8a-1 protein;
ii) a Cas3 comprising Cas3′ and Cas3″;
iii) a Casa protein;
iv) a Cas7a protein;
vi) a Cas11a protein;
wherein at least one of said proteins of i)-vi) comprises an amino acid sequence that is at least 85% identical to said protein that is encoded by a Type I-A CRISPR system, said system optionally being Pyrococcus furiosus (P. furiosus);
vi) optionally a heterologous Cas6 protein that is not encoded by P. furiosus
vi) a heterologous Cas6 protein that is not encoded by P. furiosus, and
vii) a chimeric guide RNA comprising a P. furiosus encoded 5′-handle sequence, and a 3′-handle sequence that is not encoded by P. furiosus, wherein optionally, a precursor to the chimeric guide RNA is not produced using P. furiosus, and wherein the guide RNA is directed to a target sequence that is comprised by a chromosome or an extrachromosomal element;
such that the Type I-A CRISPR system participates in degrading a DNA strand comprised by the segment of the chromosome or the extrachromosomal element that is linked to the target sequence.

14. The method of claim 13, wherein the degrading comprises bi-directional degradation of the DNA strand comprised by the segment of the chromosome or the extrachromosomal element.

15. The method of claim 14, wherein the guide RNA is directed to a target sequence that is comprised by the chromosome.

16. The method of claim 14, wherein the target sequence is linked to a cancer associated gene.

17. The method of claim 14, wherein the cancer associated gene comprises a oncogenic mutation.

18. The method of claim 14, wherein the target sequence is linked to a single nucleotide polymorphism (SNP), or a trinucleotide repeat expansion, wherein said SNP or trinucleotide repeat expansion is associated with a disorder.

19. The method of claim 1, wherein the target sequence is linked to a mutation in a non-coding sequence of a gene, wherein gene is associated with a disorder, wherein deletion of the mutation results in inactivation of the gene.

20. The method of any one of claims 13-19, wherein at least one component of the Type I-A CRISPR system is introduced into the cells by expression of a recombinant polynucleotide that encodes said at least one component.

21. The method of any one of claims 13-19, wherein the cells are present in an individual.

22. An in vitro assay for detecting the presence, absence, or amount of a single or double stranded polynucleotide comprising a target sequence in a sample, the assay comprising:
combining with a test sample:
A)
i) a Cas8a-1 protein;
ii) a Cas3 comprising Cas3′ and Cas3″;
iii) a Cas5a protein;
iv) a Cas7a protein;
vi) a Cas11a protein; and
wherein at least one of said proteins of i)-vi) comprises an amino acid sequence that is at least 85% identical to said protein that is encoded by a Type I-A CRISPR system, said system optionally being Pyrococcus furiosus (P. furiosus);
B)
vii) a chimeric guide RNA comprising a Pfu 5′-handle, and optionally a 3′-handle that is not encoded by a P. furiosus CRISPR array sequence, said chimeric guide RNA being functional with the system of A), and wherein optionally, a precursor to the chimeric guide RNA is not produced using P. furiosus, and wherein the chimeric guide RNA is targeted to the target sequence in the single or the double stranded polynucleotide and selectively binds to said target sequence if said target sequence is present in the sample; and
C)
viii) a single stranded DNA reporter comprising a detectable label, wherein a signal from said label is detectable due to cleavage of the ssDNA reporter by the Cas3 complex; and
detecting a signal from the single stranded DNA reporter of C) if the single or double stranded polynucleotide target is present the sample and the single stranded DNA reporter is cleaved by the Cas3 complex.

23. The assay claim 22, wherein the polynucleotide comprising the target sequence is present in a double stranded DNA.

24. The assay of claim 22, wherein the polynucleotide comprising the target sequence is present in a single stranded DNA.

25. The assay of claim 22, wherein the polynucleotide comprising the target sequence is present in a single stranded RNA.

26. The assay of any one of claims 22-25, wherein cleavage of the single stranded DNA reporter is performed at a temperature of above 37° C., and optionally at a temperature between 37° C. and 85° C.

27. The assay of any one of claims 22-25, wherein combining the sample with A), B) and C) are present in a single reaction container, and wherein optionally, the single stranded polynucleotide comprising the target sequence is not amplified.

28. The assay of any one of claims 22-25, wherein the detectable label comprises a fluorescent moiety that is quenched by a quencher moiety attached to the single stranded DNA reporter, and wherein said quencher moiety quenches a fluorescent signal from the fluorescent moiety until the single stranded DNA reporter is cleaved.

29. The assay of any one of claims 22-25, wherein the chimeric guide RNA is produced using a system that includes a Cas6 protein that is not encoded by P. furiosus.

30. The assay of any one of claims 22-25, wherein detecting the signal from the single stranded DNA reporter is performed in a container or a lateral flow device.

31. A kit comprising an isolated or recombinantly proteins of claim 23 A), and optionally a single stranded DNA reporter comprising a detectable label.

32. The kit of claim 31, further comprising the single stranded DNA reporter comprising the detectable label.
